(19) United States
(12) Patent Application Publication
(10) Pub. No.: US 2017/0042834 A1
(43) Pub. Date: Feb. 16, 2017

(54) ION CHANNEL ACTIVATORS AND METHODS OF USE

(71) Applicant: FLEX PHARMA, INC., Boston, MA (US)

(72) Inventors: Christoph Westphal, Boston, MA (US); Jennifer Cermak, Berlin, MA (US); Roderic O. Cole, Plaistow, NH (US); Glenn F. Short, III, Scituate, MA (US); Robert Perni, Marlborough, MA (US); Sridevi Ponduru, Belmont, MA (US)

(21) Appl. No.: 15/304,418

(22) PCT Filed: Apr. 14, 2015
(86) PCT No.: PCT/US15/25811

§ 371 Date: Oct. 14, 2016

Related U.S. Application Data
(60) Provisional application No. 62/073,258, filed on Oct. 31, 2014.
(60) Provisional application No. 62/073,131, filed on Oct. 31, 2014.
(60) Provisional application No. 61/979,349, filed on Apr. 14, 2014.

(51) Int. Cl. A61K 31/12; A61K 9/48; A61K 36/81; A61K 36/54; A23L 2/52; A61K 31/165; A61K 31/11; A61K 49/00; A61B 5/00; A61K 9/00; A61K 36/9068

(52) U.S. Cl. CPC A61K 31/12; A61K 9/0053; A61K 9/48; A61K 36/81; A61K 36/54; A61K 36/9068; A61K 9/0095; A61K 31/165; A61K 31/11; A61K 49/0004; A61B 5/4848; A61B 5/4519; A23L 2/52; A23V 2002/00

(57) ABSTRACT

The present invention relates to compositions of ion channel activators and methods of preparation, formulation, and the medical use of these compositions. In one aspect, the present invention features a composition formulated for oral administration, said composition comprising an effective amount of an ion channel activator (e.g., a TRPV1 channel activator, a TRPA1 channel activator, an ASIC channel activator, or combination thereof).

### CLAIM OF PRIORITY

This application claims priority to U.S. Provisional Application No. 61/979,349, filed Apr. 14, 2014, U.S. Provisional Application No. 62/073,131, filed Oct. 31, 2014, and U.S. Provisional Application No. 62/073,258, filed Oct. 31, 2014, the entire contents of each of which is incorporated herein by reference.

### FIELD OF THE INVENTION

The invention relates to compositions of ion channel activators and methods of preparation, formulation, and the medical use of these compositions.

### BACKGROUND

Transient receptor potential (TRP) channels are nonselective cation channels that function as cellular sensors that respond to and integrate diverse signals, including temperature, mechanical stress, exogenous chemicals, and endogenous chemicals, such as intracellular and extracellular messengers. These channels are involved in multiple functions, including pain, temperature, and mechanical sensation, calcium and magnesium homeostasis, lysosomal function, cardiovascular regulation, and control of cell growth and proliferation.

Acid-sensing ion channels (ASIC) are neuronal voltage-insensitive cationic channels that are activated by extracellular protons. ASIC channels are primarily expressed in the nervous system, and conduct mostly Na⁺. Because of their involvement in multiple cellular processes, TRP and ASIC channels play a major contributing role in a wide variety of neurological disorders, including neuropathic pain, cell injury during cerebral ischemia, and mucolipidosis type IV.

There exists a need in the art for improved methods and compositions for treating peripheral nervous system conditions (e.g., peripheral neuropathy), central nervous system conditions, muscle conditions and disorders (e.g., fibromyalgia, muscle spasms and cramps (e.g., nocturnal cramps), painful muscle contractions (e.g., a muscle contraction of the head or neck), neuromuscular disorders (e.g., motor neuron disease) or dystonia (e.g., cervical dystonia, blepharospasm, back spasms, or leg cramps due to spinal stenosis)), connective tissue diseases (e.g., degenerative joint disease), throat conditions (e.g., dysphagia or spasmodic dysphonias), tactile sensitivity, electrolyte imbalance and/or vitamin deficiency, respiratory conditions (e.g., asthma), cough, and sarcoidosis. As shown herein, compositions that include activators of ion channels (e.g., TRP or ASIC channels) may be useful to treat the above-mentioned conditions.

### SUMMARY OF THE INVENTION

In one aspect, the present invention features a composition formulated for oral administration, said composition comprising an effective amount of an ion channel activator (e.g., a TRPV1 channel activator, a TRPA1 channel activator, an ASIC channel activator, or combination thereof). In some embodiments, the composition comprises a plurality of (e.g., two or three) ion channel activators (e.g., TRPV1 channel activators, TRPA1 channel activators, ASIC channel activators, or combinations thereof). In some embodiments, the composition further comprises a pharmaceutically acceptable excipient. In some embodiments, the composition further comprises a plurality of pharmaceutically acceptable excipients.

In some embodiments, the composition is formulated for modified release (e.g., delayed release, extended release, or rapid release) of said ion channel activator (e.g., TRPV1 channel activator, TRPA1 channel activator, ASIC channel activator, or combination thereof). In some embodiments, said pharmaceutically acceptable excipient comprises an agent for modified release (e.g., delayed release, extended release, or rapid release) of an ion channel activator (e.g., a TRPV1 channel activator, a TRPA1 channel activator, an ASIC channel activator, or combination thereof), such that, when orally administered to a subject, the ion channel activator (e.g., TRPV1 channel activator, TRPA1 channel activator, ASIC channel activator, or combination thereof) is not substantially released in the stomach of said subject. In some embodiments, the agent for modified release (e.g., delayed release, extended release, or rapid release) is selected from the group consisting of: hydroxypropyl cellulose, hydroxypropyl methyl cellulose, methyl cellulose, carboxymethyl cellulose, and mixtures thereof.

In some embodiments, said pharmaceutically acceptable excipient comprises a coating. In some embodiments, said coating is selected from the group consisting of: enteric coatings, sugar coatings, and polymeric coatings. In some embodiments, said ion channel activator (e.g., TRPV1 channel activator, TRPA1 channel activator, ASIC channel activator, or combination thereof) is embedded in biodegradable microparticles or nanoparticles for sustained release.

In some embodiments, the composition further comprises a formulation base. In some embodiments, the formulation base comprises an oil and a lipophilic additive. In some embodiments, said oil is selected from the group consisting of: vegetable oil, mineral oil, soya oil, sunflower oil, corn oil, olive oil, nut oil, and liquid paraffin. In some embodiments, said lipophilic additive is selected from the group consisting of: polyethylene glycol, fatty acid mono-, di-, or triglycerides, palmitic acid, stearic acid, behenic acid, polyethylene glycol fatty acid esters, candelilla wax, carnauba wax, polyethylene oxide wax, and petroleum wax. In some embodiments, the composition further comprises a coloring agent, a dissolving agent, a flavoring agent, a sweetener, a viscosity modifier, an electrolyte, a vitamin, a mineral, an antioxidant, or a preservative.

In any embodiment of the invention, the TRPV1 channel activator is a capsaicinoid, a capsinoid, oleoylethanolamide, N-oleoyldopamine, 3-methyl-N-oleoyldopamine, oleamide, capsiate, a 1-monoacylglycerol having C18 and C20 unsaturated and C8-C12 saturated fatty acids, a 2-monoacylglycerol having C18 and C20 unsaturated fatty acids, miogadial, miogatrial, polygodial, a terpenoid with an alpha,beta-unsaturated 1,4-dialdehyde moiety, sanshool, evodiamine, acesulfame-K, cyclamate, CuSO₄, ZnSO₄, FeSO₄, arvanil, anandamide, N-arachidonoyl-dopamine, flufenamic acid dopamide, a dopamine amide of fenamic acid, 4-hydroxynonenal, 1-[2-(1-adamantyl)ethyl]-1-pentyl-3-[3-(4-pyridyl)propyl]urea, or gingerol.

In particular embodiments, the capsaicinoid is capsaicin. In some aspects of this embodiment, the TRPV1 channel activator is present from about 0.001% to about 10% (w/w) or from about 0.001% to about 10% (v/v).

In some embodiments, the TRPV1 channel activator is naturally occurring or non-naturally occurring. In some embodiments, said naturally occurring TRPV1 channel activator is selected from the group consisting of: capsaicin, dihydrocapsaicin, nordihydrocapsaicin, homodihydrocapsaicin, homocapsaicin, nonivamide, pseudocapsaicin, resiniferatoxin, tinyatoxin, capsiate, dihydrocapsiate, nordihydrocapsiate, norcapsaicin, capsiconiate, dihydrocapsiconiate and other coniferyl esters, capsiconinoid, and 3-hydroxyacetanilide.

In some embodiments, the non-naturally occurring TRPV1 channel activator is selected from the group consisting of: 4-[((6E)-8-methylnon-6-enoylamino)methyl]-2-methyoxyphenyl formate, 4-[((6E)-8-methylnon-6-enoylamino)methyl]-2-methyoxyphenyl acetate, 4-[((6E)-8-methylnon-6-enoylamino)methyl]-2-methyoxyphenyl propanoate, 4-[((6E)-8-methylnon-6-enoylamino)methyl]-2-methyoxyphenyl butanoate, 4-[((6E)-8-methylnon-6-enoylamino)methyl]-2-methoxyphenyl 2,2-dimethylpropanoate, 4-[((6E)-8-methylnon-6-enoylamino)methyl]-2-methyoxyphenyl octadecanoate, 4-[((6E)-8-methylnon-6-enoylamino)methyl]-2-methyoxyphenyl{4-[((6E)-8-methylnon-6-enoylamino)methyl]-2-methoxyphenoxy}formate, homovanillyl 8-methylnonanoate, 3-(3-methoxy-4-hydroxyphenyl)propyl 8-methylnonanoate, 8-methylnonyl homovanillate, 8-methylnonanoic acid-substituted benzyl ester derivative, heptanoyl isobutylamide, heptanoyl guaiacylamide, 7-phenylhept-6-yne-acid-4-hydroxy-3-methoxybenzylamide, dohevanil, denatonium capsaicinate, N-[4-(2-aminoethoxy)-3-methoxybenzyl]-N′-[2-(4-chlorophenyl)ethyl]thiourea, N-[4-(2-aminoethoxy)-3-methoxybenzyl]-N′-[2-(4-fluorophenyl)ethyl]thiourea, N-[4-(2-aminoethoxy)-3-methoxybenzyl]-N′-[2-(2,4-dichlorophenyl)ethyl]thiourea, N-[4-(2-aminoethoxy)-3-methoxybenzyl]-N′-[2-(4-benzyloxyphenyl)ethyl]thiourea, N-[4-(2-aminoethoxy)-3-methoxybenzyl]-N′-[2-(4-(N-octyloxy)phenyl)ethyl]thiourea, N-[4-(2-aminoethoxy)-3-methoxybenzyl]-N′-[4-N-octyloxybenzyl]thiourea, N-phenylmethylalkynamide capsaicin derivatives, N-(4-O-glycerol-3-methoxybenzyl)nonamide, N-nonanoyl vanillylamide-4-glyceryl ether, N-(4-O-acetic acid sodium)-3-methoxybenzyl-nonamide (sodium N-nonanoyl vanillylamide-4-O-acetate), N-(4-O-glycol-3-methoxybenzyl)-nonamide (N-nonanoyl vanillylamide-4-glycol ether), 20-homovanillyl-mezerein, 20-homovanillyl-12-deoxyphorbol-13-phenylacetate), civamide (N-[(4-hydroxy-3-methoxyphenyl)-methyl]-8-methyl-(Z)-6-nonemamide), nuvanil, capsavanil, olvanil, arvanil, and palvanil (N-palmitoyl-vanillamide).

In some embodiments of the invention, the TRPA1 channel activator is allyl isothiocyanate, gingerol, cinnamaldehyde, acrolein, farnesyl thiosalicylic acid, Δ₉-tetrahydrocannabinol, eugenol, a shogaol, a sanshool, allicin, diallyl sulfide, diallyl disulfide, diallyl trisulfide, or farnesyl thioacetic acid. In some aspects of this embodiment, the TRPA1 channel activator is present from about 0.001% to about 10% (w/w) or from about 0.001% to about 10% (v/v).

In some embodiments, the ASIC channel activator comprises acetic acid, phosphoric acid, citric acid, malic acid, succinic acid, lactic acid, tartaric acid, fumaric acid, or ascorbic acid. In some embodiments, the ASIC channel activator is present from about 0.001% to about 10% (w/w) or from about 0.001% to about 10% (v/v).

In some embodiments, the composition is a liquid or a solid. In some embodiments, the composition is formulated as a liquid. In some embodiments, the liquid is selected from the group consisting of emulsions, microemulsions, solutions, suspensions, syrups (e.g., syrup concentrates), linctuses, drops, sprays, and elixirs. In some embodiments, the composition is formulated as a solid. In some embodiments, the solid is selected from the group consisting of tablets, capsules, powders, crystals, pastes, gels, lozenges (e.g., liquid filled lozenges), gums, candies, chews, foodstuffs, dissolving strips, films, and semi-solid formulations. In some embodiments, said solid is a tablet or capsule. In some embodiments, said capsule is a hard or soft capsule.

In some embodiments, the ion channel activator (e.g., a TRPV1 channel activator, a TRPA1 channel activator, an ASIC channel activator, or combination thereof) comprises about 0.01% or more of a liquid formulation, e.g., about 0.02%, about 0.03%, about 0.04%, about 0.05%, about 0.075%, about 0.1%, or more. In some embodiments, the ion channel activator (e.g., a TRPV1 channel activator, a TRPA1 channel activator, an ASIC channel activator, or combination thereof) comprises about 0.1% or more of a liquid formulation, e.g., about 0.2%, about 0.3%, about 0.4%, about 0.5%, about 0.75%, about 1%, or more. In some embodiments, the ion channel activator (e.g., a TRPV1 channel activator, a TRPA1 channel activator, an ASIC channel activator, or combination thereof) comprises about 1% or more of a liquid formulation, e.g., about 2%, about 3%, about 4%, about 5%, about 7.5%, about 10%, or more. In some embodiments, the ion channel activator (e.g., a TRPV1 channel activator, a TRPA1 channel activator, an ASIC channel activator, or combination thereof) comprises between about 0.5% and 5% of a liquid formulation. In some embodiments, the ion channel activator (e.g., a TRPV1 channel activator, a TRPA1 channel activator, an ASIC channel activator, or combination thereof) comprises between about 0.5% and 2.5% of a liquid formulation. In some embodiments, the ion channel activator (e.g., a TRPV1 channel activator, a TRPA1 channel activator, an ASIC channel activator, or combination thereof) comprises about 10% or more of a liquid formulation, e.g., about 20%, about 25%, about 30%, about 35%, about 40%, about 45%, about 50%, or more.

In some embodiments, the composition comprises a liquid formulation (e.g., an emulsion, microemulsion, solution, suspension, syrup (e.g., syrup concentrate), linctus, drop, or elixir) and an effective amount of the liquid formulation is at least about 1 mL, about 2 mL, about 4 mL, about 6 mL, about 8 mL, about 10 mL, about 12 mL, about 14 mL, about 16 mL, about 18 mL, about 20 mL, about 22.5 mL, about 25 mL, or more. In some embodiments, an effective amount of the liquid formulation is between about 1 mL and about 10 mL. In some embodiments, an effective amount of the liquid formulation is between about 5 mL and about 10 mL. In some embodiments, an effective amount of the liquid formulation is between about 10 mL and about 25 mL.

In some embodiments, the composition comprises a liquid formulation (e.g., an emulsion, microemulsion, solution, suspension, syrup (e.g., syrup concentrate), linctus, drop, or elixir) and an effective amount of the liquid formulation is at least about 25 mL, about 30 mL, about 35 mL, about 40 mL, about 45 mL, about 50 mL, about 60 mL, about 75 mL, about 100 mL, about 150 mL, about 200 mL, about 300 mL, about 400 mL, about 500 mL, about 600 mL, about 750 mL, about 1000 mL, or more. In some embodiments, an effective amount of the liquid formulation is between about 25 mL and about 100 mL. In some embodiments, an effective amount of the liquid formulation is between about 50 mL and about 500 mL. In some embodiments, an effective amount of the liquid formulation is between about 100 mL and about 1000 mL.

In some embodiments, the composition comprises a liquid formulation (e.g., an emulsion, microemulsion, solution, suspension, syrup (e.g., syrup concentrate), linctus, drop, or elixir) and an effective amount of the liquid formulation is at least about 1 fluid ounce, about 2 fluid ounces, about 3 fluid ounces, about 4 fluid ounces, about 5 fluid ounces, about 6 fluid ounces, about 7 fluid ounces, about 8 fluid ounces, about 9 fluid ounces, about 10 fluid ounces, about 11 fluid ounces, about 12 fluid ounces, or more. In some embodiments, an effective amount of the liquid formulation is at least about 12 fluid ounces, about 16 fluid ounces, about 20 fluid ounces, about 24 fluid ounces, about 32 fluid ounces, about 40 fluid ounces, about 48 fluid ounces, about 56 fluid ounces, about 64 fluid ounces, or more.

In some embodiments, the composition comprises a solid formulation (e.g., a tablet, capsule, powder, crystal, paste, gel, lozenge (e.g., liquid filled lozenge), gum, candy, foodstuff, dissolving strip, film, or semi-solid formulation) and an effective amount of the solid formulation is about 0.5 mg, about 1 mg, about 10 mg, about 25 mg, about 50 mg, about 100 mg, about 250 mg, about 500 mg, about 750 mg, about 1 g, about 2 g, about 5 g, about 10 g, or more. In some embodiments, an effective amount of the solid formulation is between about 0.5 mg and about 100 mg. In some embodiments, an effective amount of the solid formulation is between about 100 mg and about 500 mg. In some embodiments, an effective amount of the solid formulation is between about 500 mg and about 1000 mg.

In some embodiments, the composition comprising an ion channel activator (e.g., a TRPV1 channel activator, a TRPA1 channel activator, an ASIC channel activator, or combination thereof) is formulated for use once a day. In some embodiments, the composition is formulated for use at least about 1 time per day, about 2 times per day, about 3 times per day, about 4 times per day, about 5 times per day, or more. In some embodiments, the composition is formulated for use about 1-3 times per day. In some embodiments, the composition is formulated for use for a period of about 1 day, about 2 days, about 3 days, about 4 days, about 5 days, about 6 days, about 1 week, about 2 weeks, about 3 weeks, about 4 weeks, about 5 weeks, about 6 weeks, about 2 months, about 3 months, about 4 months, about 5 months, about 6 months, or more.

In another aspect, the invention features a composition formulated for oral administration, said composition comprising an effective amount of an ion channel activator (e.g., a TRPV1 channel activator, a TRPA1 channel activator, an ASIC channel activator, or combination thereof), and a pharmaceutically acceptable excipient, wherein said composition is a liquid or solid, and wherein said composition is formulated for delayed release of said ion channel activator (e.g., TRPV1 channel activator, TRPA1 channel activator, ASIC channel activator, or combination thereof). In some embodiments, said composition comprises a plurality of (e.g., two or three) ion channel activators (e.g., TRPV1 channel activators, TRPA1 channel activators, ASIC channel activators, or combinations thereof), and a pharmaceutically acceptable excipient. In some embodiments, said composition comprises an ion channel activator (e.g., a TRPV1 channel activator, a TRPA1 channel activator, an ASIC channel activator, or combination thereof), and a plurality of pharmaceutically acceptable excipients.

In some embodiments, said pharmaceutically acceptable excipient comprises an agent for delayed release of an ion channel activator (e.g., a TRPV1 channel activator, a TRPA1 channel activator, an ASIC channel activator, or combination thereof), such that, when orally administered to a subject, the ion channel activator (e.g., TRPV1 channel activator, TRPA1 channel activator, ASIC channel activator, or combination thereof) is not substantially released in the stomach of said subject. In some embodiments, the agent for delayed release is selected from the group consisting of: hydroxypropyl cellulose, hydroxypropyl methyl cellulose, methyl cellulose, carboxymethyl cellulose, and mixtures thereof.

In some embodiments, said pharmaceutically acceptable excipient comprises a coating. In some embodiments, said coating is selected from the group consisting of: enteric coatings, sugar coatings, and polymeric coatings. In some embodiments, said ion channel activator (e.g., TRPV1 channel activator, TRPA1 channel activator, ASIC channel activator, or combination thereof) is embedded in biodegradable microparticles or nanoparticles for sustained release.

In some embodiments, the composition further comprises a formulation base. In some embodiments, the formulation base comprises an oil and a lipophilic additive. In some embodiments, said oil is selected from the group consisting of: vegetable oil, mineral oil, soya oil, sunflower oil, corn oil, olive oil, nut oil, and liquid paraffin. In some embodiments, said lipophilic additive is selected from the group consisting of: polyethylene glycol, fatty acid mono-, di-, or triglycerides, palmitic acid, stearic acid, behenic acid, polyethylene glycol fatty acid esters, candelilla wax, carnauba wax, polyethylene oxide wax, and petroleum wax. In some embodiments, the composition further comprises a coloring agent, a dissolving agent, a flavoring agent, a sweetener, a viscosity modifier, an electrolyte, a vitamin, a mineral, an antioxidant, or a preservative.

In some embodiments, the composition is formulated as a liquid. In some embodiments, the liquid is selected from the group consisting of emulsions, microemulsions, solutions, suspensions, syrups (e.g., syrup concentrates), linctuses, drops, sprays, and elixirs. In some embodiments, the composition is formulated as a solid. In some embodiments, the solid is selected from the group consisting of tablets, capsules, powders, crystals, pastes, gels, lozenges (e.g., liquid filled lozenges), gums, candies, chews, foodstuffs, dissolving strips, films, and semi-solid formulations. In some embodiments, said solid is a tablet or capsule. In some embodiments, said capsule is a hard or soft capsule.

In some embodiments, the composition comprising an ion channel activator (e.g., a TRPV1 channel activator, a TRPA1 channel activator, an ASIC channel activator, or combination thereof) is formulated for use once a day. In some embodiments, the composition is formulated for use at least about 1 time per day, about 2 times per day, about 3 times per day, about 4 times per day, about 5 times per day, or more. In some embodiments, the composition is formulated for use about 1-3 times per day. In some embodiments, the composition is formulated for use for a period of about 1 day, about 2 days, about 3 days, about 4 days, about 5 days, about 6 days, about 1 week, about 2 weeks, about 3 weeks, about 4 weeks, about 5 weeks, about 6 weeks, about 2 months, about 3 months, about 4 months, about 5 months, about 6 months, or more.

In any embodiment of the invention, the TRPV1 channel activator is a capsaicinoid, a capsinoid, oleoylethanolamide, N-oleoyldopamine, 3-methyl-N-oleoyldopamine, oleamide, capsiate, a 1-monoacylglycerol having C18 and C20 unsaturated and C8-C12 saturated fatty acid, a 2-monoacylglycerol having C18 and C20 unsaturated fatty acids, miogadial, miogatrial, polygodial, a terpenoid with an alpha,beta-unsaturated 1,4-dialdehyde moiety, sanshool, evodiamine, acesulfame-K, cyclamate, CuSO₄, ZnSO₄, FeSO₄, arvanil, anandamide, N-arachidonoyl-dopamine, flufenamic acid dopamide, a dopamine amide of fenamic acid, 4-hydroxynonenal, 1-[2-(1-adamantyl)ethyl]-1-pentyl-3-[3-(4-pyridyl)propyl]urea, or gingerol.

In particular embodiments, the capsaicinoid is capsaicin. In some aspects of this embodiment, the TRPV1 channel activator is present from about 0.001% to about 10% (w/w) or from about 0.001% to about 10% (v/v).

In some embodiments, the TRPV1 channel activator is naturally occurring or non-naturally occurring. In some embodiments, said naturally occurring TRPV1 channel activator is selected from the group consisting of: capsaicin, dihydrocapsaicin, nordihydrocapsaicin, homodihydrocapsaicin, homocapsaicin, nonivamide, pseudocapsaicin, resiniferatoxin, tinyatoxin, capsiate, dihydrocapsiate, nordihydrocapsiate, norcapsaicin, capsiconiate, dihydrocapsiconiate and other coniferyl esters, capsiconinoid, and 3-hydroxyacetanilide.

In some embodiments, the non-naturally occurring TRPV1 channel activator is selected from the group consisting of: 4-[((6E)-8-methylnon-6-enoylamino)methyl]-2-methyoxyphenyl formate, 4-[((6E)-8-methylnon-6-enoylamino)methyl]-2-methyoxyphenyl acetate, 4-[((6E)-8-methylnon-6-enoylamino)methyl]-2-methyoxyphenyl propanoate, 4-[((6E)-8-methylnon-6-enoylamino)methyl]-2-methyoxyphenyl butanoate, 4-[((6E)-8-methylnon-6-enoylamino)methyl]-2-methoxyphenyl 2,2-dimethylpropanoate, 4-[((6E)-8-methylnon-6-enoylamino)methyl]-2-methyoxyphenyl octadecanoate, 4-[((6E)-8-methylnon-6-enoylamino)methyl]-2-methyoxyphenyl{4-[((6E)-8-methylnon-6-enoylamino)methyl]-2-methoxyphenoxy}formate, homovanillyl 8-methylnonanoate, 3-(3-methoxy-4-hydroxyphenyl)propyl 8-methylnonanoate, 8-methylnonyl homovanillate, 8-methylnonanoic acid-substituted benzyl ester derivative, heptanoyl isobutylamide, heptanoyl guaiacylamide, 7-phenylhept-6-yne-acid-4-hydroxy-3-methoxybenzylamide, dohevanil, denatonium capsaicinate, N-[4-(2-aminoethoxy)-3-methoxybenzyl]-N′-[2-(4-chlorophenyl)ethyl]thiourea, N-[4-(2-aminoethoxy)-3-methoxybenzyl]-N′-[2-(4-fluorophenyl)ethyl]thiourea, N-[4-(2-aminoethoxy)-3-methoxybenzyl]-N′-[2-(2,4-dichlorophenyl)ethyl]thiourea, N-[4-(2-aminoethoxy)-3-methoxybenzyl]-N′-[2-(4-benzyloxyphenyl)ethyl]thiourea, N-[4-(2-aminoethoxy)-3-methoxybenzyl]-N′-[2-(4-(N-octyloxy)phenyl)ethyl]thiourea, N-[4-(2-aminoethoxy)-3-methoxybenzyl]-N′-[4-N-octyloxybenzyl]thiourea, N-phenylmethylalkynamide capsaicin derivatives, N-(4-O-glycerol-3-methoxybenzyl)-nonamide, N-nonanoyl vanillylamide-4-glyceryl ether, N-(4-O-acetic acid sodium)-3-methoxybenzyl-nonamide (sodium N-nonanoyl vanillylamide-4-O-acetate), N-(4-O-glycol-3-methoxybenzyl)-nonamide (N-nonanoyl vanillylamide-4-glycol ether), 20-homovanillyl-mezerein, 20-homovanillyl-12-deoxyphorbol-13-phenylacetate), civamide (N-[(4-hydroxy-3-methoxyphenyl)-methyl]-8-methyl-(Z)-6-nonemamide), nuvanil, capsavanil, olvanil, arvanil, and palvanil (N-palmitoyl-vanillamide).

In further embodiments of the invention, the TRPA1 channel activator is allyl isothiocyanate, gingerol, cinnamaldehyde, acrolein, farnesyl thiosalicylic acid, Δ₉-tetrahydrocannabinol, eugenol, a shogaol, a sanshool, allicin, diallyl sulfide, diallyl disulfide, diallyl trisulfide, or farnesyl thioacetic acid. In some aspects of this embodiment, the TRPA1 channel activator is present from about 0.001% to about 10% (w/w) or from about 0.001% to about 10% (v/v).

In some embodiments, the ASIC channel activator comprises acetic acid, phosphoric acid, citric acid, malic acid, succinic acid, lactic acid, tartaric acid, fumaric acid, or ascorbic acid. In some embodiments, the ASIC channel activator is present from about 0.001% to about 10% (w/w) or from about 0.001% to about 10% (v/v).

In some embodiments, the composition is capable of reducing gastrointestinal side effects.

In another aspect, the invention features a composition comprising an effective amount of an ion channel activator (e.g., a TRPV1 channel activator, a TRPA1 channel activator, an ASIC channel activator, or combination thereof), a formulation base, and a pharmaceutically acceptable excipient. In some embodiments, said composition comprises a plurality of (e.g., two or three) ion channel activators (e.g., TRPV1 channel activators, TRPA1 channel activators, ASIC channel activators, or combinations thereof), a formulation base, and a pharmaceutically acceptable excipient. In some embodiments, said composition comprises an ion channel activator (e.g., a TRPV1 channel activator, a TRPA1 channel activator, an ASIC channel activator, or combination thereof), a formulation base, and a plurality of pharmaceutically acceptable excipients.

In some embodiments, the formulation base comprises an oil and a lipophilic additive. In some embodiments, said oil is selected from the group consisting of: vegetable oil, mineral oil, soya oil, sunflower oil, corn oil, olive oil, nut oil, and liquid paraffin. In some embodiments, said lipophilic additive is selected from the group consisting of: polyethylene glycol, fatty acid mono-, di-, or triglycerides, palmitic acid, stearic acid, behenic acid, polyethylene glycol fatty acid esters, candelilla wax, carnauba wax, polyethylene oxide wax, and petroleum wax. In some embodiments, the composition further comprises a coloring agent, a dissolving agent, a flavoring agent, a sweetener, a viscosity modifier, an electrolyte, a vitamin, a mineral, an antioxidant, or a preservative.

In some embodiments, the composition is formulated as a liquid or a solid. In some embodiments, the composition is formulated as a liquid. In some embodiments, the liquid is selected from the group consisting of emulsions, microemulsions, solutions, suspensions, syrups (e.g., syrup concentrates), linctuses, drops, sprays, and elixirs. In some embodiments, the composition is formulated as a solid. In some embodiments, the solid is selected from the group consisting of tablets, capsules, powders, crystals, pastes, gels, lozenges (e.g., liquid filled lozenges), gums, candies, chews, foodstuffs, dissolving strips, films, and semi-solid formulations. In some embodiments, said solid is a tablet or capsule. In some embodiments, said capsule is a hard or soft capsule.

In some embodiments, the composition comprising an ion channel activator (e.g., a TRPV1 channel activator, a TRPA1 channel activator, an ASIC channel activator, or combination thereof) is formulated for use once a day. In some embodiments, the composition is formulated for use at least about 1 time per day, about 2 times per day, about 3 times per day, about 4 times per day, about 5 times per day, or more. In some embodiments, the composition is formulated for use about 1-3 times per day. In some embodiments, the composition is formulated for use for a period of about 1 day, about 2 days, about 3 days, about 4 days, about 5 days, about 6 days, about 1 week, about 2 weeks, about 3 weeks, about 4 weeks, about 5 weeks, about 6 weeks, about 2 months, about 3 months, about 4 months, about 5 months, about 6 months, or more.

In any embodiment of the invention, the TRPV1 channel activator is a capsaicinoid, a capsinoid, oleoylethanolamide, N-oleoyldopamine, 3-methyl-N-oleoyldopamine, oleamide, capsiate, a 1-monoacylglycerol having C18 and C20 unsaturated and C8-C12 saturated fatty acid, a 2-monoacylglycerol having C18 and C20 unsaturated fatty acids, miogadial, miogatrial, polygodial, a terpenoid with an alpha,beta-unsaturated 1,4-dialdehyde moiety, sanshool, evodiamine, acesulfame-K, cyclamate, CuSO₄, ZnSO₄, FeSO₄, arvanil, anandamide, N-arachidonoyl-dopamine, flufenamic acid dopamide, a dopamine amide of fenamic acid, 4-hydroxynonenal, 1-[2-(1-adamantyl)ethyl]-1-pentyl-3-[3-(4-pyridyl)propyl]urea, or gingerol.

In particular embodiments, the capsaicinoid is capsaicin. In some aspects of this embodiment, the TRPV1 channel activator is present from about 0.001% to about 10% (w/w) or from about 0.001% to about 10% (v/v).

In some embodiments, the TRPV1 channel activator is naturally occurring or non-naturally occurring. In some embodiments, said naturally occurring TRPV1 channel activator is selected from the group consisting of: capsaicin, dihydrocapsaicin, nordihydrocapsaicin, homodihydrocapsaicin, homocapsaicin, nonivamide, pseudocapsaicin, resiniferatoxin, tinyatoxin, capsiate, dihydrocapsiate, nordihydrocapsiate, norcapsaicin, capsiconiate, dihydrocapsiconiate and other coniferyl esters, capsiconinoid, and 3-hydroxyacetanilide.

In some embodiments, the non-naturally occurring TRPV1 channel activator is selected from the group consisting of: 4-[((6E)-8-methylnon-6-enoylamino)methyl]-2-methyoxyphenyl formate, 4-[((6E)-8-methylnon-6-enoylamino)methyl]-2-methyoxyphenyl acetate, 4-[((6E)-8-methylnon-6-enoylamino)methyl]-2-methyoxyphenyl propanoate, 4-[((6E)-8-methylnon-6-enoylamino)methy]-2-methyoxyphenyl butan oate, 4-[((6E)-8-methylnon-6-enoylamino)methyl]-2-methoxyphenyl 2,2-dimethylpropanoate, 4-[((6E)-8-methylnon-6-enoylamino)methyl]-2-methyoxyphenyl octadecanoate, 4-[((6E)-8-methylnon-6-enoylamino)methyl]-2-methyoxyphenyl{4-[((6E)-8-methylnon-6-enoylamino)methyl]-2-methoxyphenoxy}formate, homovanillyl 8-methylnonanoate, 3-(3-methoxy-4-hydroxyphenyl)propyl 8-methylnonanoate, 8-methylnonyl homovanillate, 8-methylnonanoic acid-substituted benzyl ester derivative, heptanoyl isobutylamide, heptanoyl guaiacylamide, 7-phenylhept-6-yne-acid-4-hydroxy-3-methoxybenzylamide, dohevanil, denatonium capsaicinate, N-[4-(2-aminoethoxy)-3-methoxybenzyl]-N′-[2-(4-chlorophenyl)ethyl]thiourea, N-[4-(2-aminoethoxy)-3-methoxybenzyl]-N′-[2-(4-fluorophenyl)ethyl]thiourea, N-[4-(2-aminoethoxy)-3-methoxybenzyl]-N′-[2-(2,4-dichlorophenyl)ethyl]thiourea, N-[4-(2-aminoethoxy)-3-methoxybenzyl]-N′-[2-(4-benzyloxyphenyl)ethyl]thiourea, N-[4-(2-aminoethoxy)-3-methoxybenzyl]-N′-[2-(4-(n-octyloxy)phenyl)ethyl]thiourea, N-[4-(2-aminoethoxy)-3-methoxybenzyl]-N′-[4-N-octyloxybenzyl]thiourea, N-phenylmethylalkynamide capsaicin derivatives, N-(4-O-glycerol-3-methoxybenzyl)-nonamide, N-nonanoyl vanillylamide-4-glyceryl ether, N-(4-O-acetic acid sodium)-3-methoxybenzyl-nonamide (sodium N-nonanoyl vanillylamide-4-O-acetate), N-(4-O-glycol-3-methoxybenzyl)-nonamide (N-nonanoyl vanillylamide-4-glycol ether), 20-homovanillyl-mezerein, 20-homovanillyl-12-deoxyphorbol-13-phenylacetate), civamide (N-[(4-hydroxy-3-methoxyphenyl)-methyl]-8-methyl-(Z)-6-nonemamide), nuvanil, capsavanil, olvanil, arvanil, and palvanil (N-palmitoyl-vanillamide).

In further embodiments of the invention, the TRPA1 channel activator is allyl isothiocyanate, gingerol, cinnamaldehyde, acrolein, farnesyl thiosalicylic acid, Δ₉-tetrahydrocannabinol, eugenol, a shogaol, a sanshool, allicin, diallyl sulfide, diallyl disulfide, diallyl trisulfide, or farnesyl thioacetic acid. In some aspects of this embodiment, the TRPA1 channel activator is present from about 0.001% to about 10% (w/w) or from about 0.001% to about 10% (v/v).

In some embodiments, the ASIC channel activator comprises acetic acid, phosphoric acid, citric acid, malic acid, succinic acid, lactic acid, tartaric acid, fumaric acid, or ascorbic acid. In some embodiments, the ASIC channel activator is present from about 0.001% to about 10% (w/w) or from about 0.001% to about 10% (v/v).

In some embodiments, the composition is capable of reducing gastrointestinal side effects.

In another aspect, the invention features a composition formulated for oral administration to a subject, said composition comprising an effective amount of an ion channel activator (e.g., a TRPV1 channel activator, a TRPA1 channel activator, an ASIC channel activator, or combination thereof) and a pharmaceutically acceptable excipient, wherein upon administration, the ion channel activator has a residence time of greater than about 5 seconds in the mouth of the subject. In some embodiments, said composition comprises a plurality of (e.g., two or three) ion channel activators (e.g., TRPV1 channel activators, TRPA1 channel activators, ASIC channel activators, or combinations thereof) and a pharmaceutically acceptable excipient. In some embodiments, said composition comprises an ion channel activator (e.g., a TRPV1 channel activator, a TRPA1 channel activator, an ASIC channel activator, or combination thereof) and a plurality of pharmaceutically acceptable excipients.

In some embodiments, the ion channel activator (e.g., a TRPV1 channel activator, a TRPA1 channel activator, an ASIC channel activator, or combination thereof) has a residence time of greater than about 5 seconds in the mouth of a subject, e.g., greater than about 6 seconds, about 7 seconds, about 8 seconds, about 9 seconds, about 10 seconds, about 11 seconds, about 12 seconds, about 13 seconds, about 14 seconds, about 15 seconds, about 20 seconds, about 25 seconds, about 30 seconds, about 45 seconds, about 60 seconds, about 90 seconds, about 2 minutes, about 3 minutes, about 4 minutes, about 5 minutes, or more. In some embodiments, the ion channel activator (e.g., a TRPV1 channel activator, a TRPA1 channel activator, an ASIC channel activator, or combination thereof) has a residence time in the mouth of a subject between about 5 seconds and about 2 minutes. In some embodiments, the ion channel activator (e.g., a TRPV1 channel activator, a TRPA1 channel activator, an ASIC channel activator, or combination thereof) has a residence time in the mouth of a subject between about 5 seconds and about 60 seconds. In some embodiments, the ion channel activator (e.g., a TRPV1 channel activator, a TRPA1 channel activator, an ASIC channel activator, or combination thereof) has a residence time in the mouth of a subject between about 5 seconds and about 30 seconds.

In some embodiments, the ion channel activator (e.g., a TRPV1 channel activator, a TRPA1 channel activator, an ASIC channel activator, or combination thereof) has a residence time of greater than about 60 seconds in the mouth of a subject, e.g., greater than about 90 seconds, about 2 minutes, about 3 minutes, about 4 minutes, about 5 minutes, about 6 minutes, about 7 minutes, about 8 minutes, about 9 minutes, about 10 minutes, or more. In some embodiments, the ion channel activator (e.g., a TRPV1 channel activator, a TRPA1 channel activator, an ASIC channel activator, or combination thereof) has a residence time in the mouth of a subject between about 60 seconds and about 5 minutes. In some embodiments, the ion channel activator (e.g., a TRPV1 channel activator, a TRPA1 channel activator, an ASIC channel activator, or combination thereof) has a residence time in the mouth of a subject between about 60 seconds and about 3 minutes. In some embodiments, the ion channel activator (e.g., a TRPV1 channel activator, a TRPA1 channel activator, an ASIC channel activator, or combination thereof) has a residence time in the mouth of a subject between about 60 seconds and about 2 minutes.

In some embodiments, the composition comprising an ion channel activator (e.g., a TRPV1 channel activator, a TRPA1 channel activator, an ASIC channel activator, or combination thereof) is formulated for use once a day. In some embodiments, the composition is formulated for use at least about 1 time per day, about 2 times per day, about 3 times per day, about 4 times per day, about 5 times per day, or more. In some embodiments, the composition is formulated for use about 1-3 times per day. In some embodiments, the composition is formulated for use for a period of about 1 day, about 2 days, about 3 days, about 4 days, about 5 days, about 6 days, about 1 week, about 2 weeks, about 3 weeks, about 4 weeks, about 5 weeks, about 6 weeks, about 2 months, about 3 months, about 4 months, about 5 months, about 6 months, or more.

In some embodiments, the composition further comprises a formulation base. In some embodiments, the formulation base comprises an oil and a lipophilic additive. In some embodiments, said oil is selected from the group consisting of: vegetable oil, mineral oil, soya oil, sunflower oil, corn oil, olive oil, nut oil, and liquid paraffin. In some embodiments, said lipophilic additive is selected from the group consisting of: polyethylene glycol, fatty acid mono-, di-, or triglycerides, palmitic acid, stearic acid, behenic acid, polyethylene glycol fatty acid esters, candelilla wax, carnauba wax, polyethylene oxide wax, and petroleum wax. In some embodiments, the composition further comprises a coloring agent, a dissolving agent, a flavoring agent, a sweetener, a viscosity modifier, an electrolyte, a vitamin, a mineral, an antioxidant, or a preservative.

In any embodiment of the invention, the TRPV1 channel activator is a capsaicinoid, a capsinoid, oleoylethanolamide, N-oleoyldopamine, 3-methyl-N-oleoyldopamine, oleamide, capsiate, a 1-monoacylglycerol having C18 and C20 unsaturated and C8-C12 saturated fatty acid, a 2-monoacylglycerol having C18 and C20 unsaturated fatty acids, miogadial, miogatrial, polygodial, a terpenoid with an alpha,beta-unsaturated 1,4-dialdehyde moiety, sanshool, evodiamine, acesulfame-K, cyclamate, CuSO₄, ZnSO₄, FeSO₄, arvanil, anandamide, N-arachidonoyl-dopamine, flufenamic acid dopamide, a dopamine amide of fenamic acid, 4-hydroxynonenal, 1-[2-(1-adamantyl)ethyl]-1-pentyl-3-[3-(4-pyridyl)propyl]urea, or gingerol.

In particular embodiments, the capsaicinoid is capsaicin. In some aspects of this embodiment, the TRPV1 channel activator is present from about 0.001% to about 10% (w/w) or from about 0.001% to about 10% (v/v).

In some embodiments, the TRPV1 channel activator is naturally occurring or non-naturally occurring. In some embodiments, said naturally occurring TRPV1 channel activator is selected from the group consisting of: capsaicin, dihydrocapsaicin, nordihydrocapsaicin, homodihydrocapsaicin, homocapsaicin, nonivamide, pseudocapsaicin, resiniferatoxin, tinyatoxin, capsiate, dihydrocapsiate, nordihydrocapsiate, norcapsaicin, capsiconiate, dihydrocapsiconiate and other coniferyl esters, capsiconinoid, and 3-hydroxyacetanilide.

In some embodiments, the non-naturally occurring TRPV1 channel is selected from the group consisting of: 4-[((6E)-8-methylnon-6-enoylamino)methyl]-2-methyoxyphenyl formate, 4-[((6E)-8-methylnon-6-enoylamino)methyl]-2-methyoxyphenyl acetate, 4-[((6E)-8-methylnon-6-enoylamino)methyl]-2-methyoxyphenyl propanoate, 4-[((6E)-8-methylnon-6-enoylamino)methyl]-2-methyoxyphenyl butanoate, 4-[((6E)-8-methylnon-6-enoylamino)methyl]-2-methoxyphenyl 2,2-dimethylpropanoate, 4-[((6E)-8-methylnon-6-enoylamino)methyl]-2-methyoxyphenyl octadecanoate, 4-[((6E)-8-methylnon-6-enoylamino)methyl]-2-methyoxyphenyl{4-[((6E)-8-methylnon-6-enoylamino)methyl]-2-methoxyphenoxy}formate, homovanillyl 8-methylnonanoate, 3-(3-methoxy-4-hydroxyphenyl)propyl 8-methylnonanoate, 8-methylnonyl homovanillate, 8-methylnonanoic acid-substituted benzyl ester derivative, heptanoyl isobutylamide, heptanoyl guaiacylamide, 7-phenylhept-6-yne-acid-4-hydroxy-3-methoxybenzylamide, dohevanil, denatonium capsaicinate, N-[4-(2-aminoethoxy)-3-methoxybenzyl]-N′-[2-(4-chlorophenyl)ethyl]thiourea, N-[4-(2-aminoethoxy)-3-methoxybenzyl]-N′-[2-(4-fluorophenyl)ethyl]thiourea, N-[4-(2-aminoethoxy)-3-methoxybenzyl]-N′-[2-(2,4-dichlorophenyl)ethyl]thiourea, N-[4-(2-aminoethoxy)-3-methoxybenzyl]-N′-[2-(4-benzyloxyphenyl)ethyl]thiourea, N-[4-(2-aminoethoxy)-3-methoxybenzyl]-N′-[2-(4-(N-octyloxy)phenyl)ethyl]thiourea, N-[4-(2-aminoethoxy)-3-methoxybenzyl]-N′-[4-N-octyloxybenzyl]thiourea, N-phenylmethylalkynamide capsaicin derivatives, N-(4-O-glycerol-3-methoxybenzyl)nonamide, N-nonanoyl vanillylamide-4-glyceryl ether, N-(4-O-acetic acid sodium)-3-methoxybenzyl-nonamide, sodium N-nonanoyl vanillylamide-4-O-acetate), N-(4-O-glycol-3-methoxybenzyl)-nonamide, N-nonanoyl vanillylamide-4-glycol ether), 20-homovanillyl-mezerein, 20-homovanillyl-12-deoxyphorbol-13-phenylacetate), civamide (N-[(4-hydroxy-3-methoxyphenyl)-methyl]-8-methyl-(Z)-6-nonemamide), nuvanil, capsavanil, olvanil, arvanil, and palvanil (N-palmitoyl-vanillamide).

In some embodiments of the invention, the TRPA1 channel activator is allyl isothiocyanate, a gingerol, cinnamaldehyde, acrolein, farnesyl thiosalicylic acid, Δ₉-tetrahydrocannabinol, eugenol, a shogaol, a sanshool, allicin, diallyl sulfide, diallyl disulfide, diallyl trisulfide, or farnesyl thioacetic acid. In some aspects of this embodiment, the TRPA1 channel activator is present from about 0.001% to about 10% (w/w) or from about 0.001% to about 10% (v/v).

In some embodiments, the ASIC channel activator comprises acetic acid, phosphoric acid, citric acid, malic acid, succinic acid, lactic acid, tartaric acid, fumaric acid, or ascorbic acid. In some embodiments, the ASIC channel activator is present from about 0.001% to about 10% (w/w) or from about 0.001% to about 1% (v/v).

In some embodiments, the composition comprising an ion channel activator (e.g., a TRPV1 channel activator, a TRPA1 channel activator, an ASIC channel activator, or combination thereof) comprises a liquid or solid formulation. In some embodiments, the liquid formulation is selected from the group consisting of emulsions, microemulsions, solutions, suspensions, syrups (e.g., syrup concentrates), linctuses, drops, sprays, and elixirs. In some embodiments, the solid formulation is selected from the group consisting of tablets, capsules, powders, crystals, pastes, gels, lozenges (e.g., liquid filled lozenges), gums, candies, chews, foodstuffs, dissolving strips, films, and semi-solid formulations. In some embodiments, said solid formulation is a tablet or capsule. In some embodiments, said capsule is a hard or soft capsule.

In some embodiments, the ion channel activator (e.g., a TRPV1 channel activator, a TRPA1 channel activator, an ASIC channel activator, or combination thereof) comprises about 0.01% or more of a liquid formulation, e.g., about 0.02%, about 0.03%, about 0.04%, about 0.05%, about 0.075%, about 0.1%, or more. In some embodiments, the ion channel activator (e.g., a TRPV1 channel activator, a TRPA1 channel activator, an ASIC channel activator, or combination thereof) comprises about 0.1% or more of a liquid formulation, e.g., about 0.2%, about 0.3%, about 0.4%, about 0.5%, about 0.75%, about 1%, or more. In some embodiments, the ion channel activator (e.g., a TRPV1 channel activator, a TRPA1 channel activator, an ASIC channel activator, or combination thereof) comprises about 1% or more of a liquid formulation, e.g., about 2%, about 3%, about 4%, about 5%, about 7.5%, about 10%, or more. In some embodiments, the ion channel activator (e.g., a TRPV1 channel activator, a TRPA1 channel activator, an ASIC channel activator, or combination thereof) comprises between about 0.5% and 5% of a liquid formulation. In some embodiments, the ion channel activator (e.g., a TRPV1 channel activator, a TRPA1 channel activator, an ASIC channel activator, or combination thereof) comprises between about 0.5% and 2.5% of a liquid formulation. In some embodiments, the ion channel activator (e.g., a TRPV1 channel activator, a TRPA1 channel activator, an ASIC channel activator, or combination thereof) comprises about 10% or more of a liquid formulation, e.g., about 20%, about 25%, about 30%, about 35%, about 40%, about 45%, about 50%, or more.

In some embodiments, the composition comprises a liquid formulation (e.g., an emulsion, microemulsion, solution, suspension, syrup (e.g., syrup concentrate), linctus, drop, or elixir) and an effective amount of the liquid formulation is at least about 1 mL, about 2 mL, about 4 mL, about 6 mL, about 8 mL, about 10 mL, about 12 mL, about 14 mL, about 16 mL, about 18 mL, about 20 mL, about 22.5 mL, about 25 mL, or more. In some embodiments, an effective amount of the liquid formulation is between about 1 mL and 10 mL. In some embodiments, an effective amount of the liquid formulation is between about 5 mL and 10 mL. In some embodiments, an effective amount of the liquid formulation is between about 10 mL and 25 mL.

In some embodiments, the composition comprises a liquid formulation (e.g., an emulsion, microemulsion, solution, suspension, syrup (e.g., syrup concentrate), linctus, drop, or elixir) and an effective amount of the liquid formulation is at least about 25 mL, about 30 mL, about 35 mL, about 40 mL, about 45 mL, about 50 mL, about 60 mL, about 75 mL, about 100 mL, about 150 mL, about 200 mL, about 300 mL, about 400 mL, about 500 mL, about 600 mL, about 750 mL, about 1000 mL, or more. In some embodiments, an effective amount of the liquid formulation is between about 25 mL and 100 mL. In some embodiments, an effective amount of the liquid formulation is between about 50 mL and 500 mL. In some embodiments, an effective amount of the liquid formulation is between about 100 mL and 1000 mL.

In some embodiments, the composition comprises a liquid formulation (e.g., an emulsion, microemulsion, solution, suspension, syrup (e.g., syrup concentrate), linctus, drop, or elixir) and an effective amount of the liquid formulation is at least about 1 fluid ounce, about 2 fluid ounces, about 3 fluid ounces, about 4 fluid ounces, about 5 fluid ounces, about 6 fluid ounces, about 7 fluid ounces, about 8 fluid ounces, about 9 fluid ounces, about 10 fluid ounces, about 11 fluid ounces, about 12 fluid ounces, or more. In some embodiments, an effective amount of the liquid formulation is at least about 12 fluid ounces, about 16 fluid ounces, about 20 fluid ounces, about 24 fluid ounces, about 32 fluid ounces, about 40 fluid ounces, about 48 fluid ounces, about 56 fluid ounces, about 64 fluid ounces, or more.

In some embodiments, the composition comprises a solid formulation (e.g., a tablet, capsule, powder, crystal, paste, gel, lozenge, gum, candy, chew, foodstuff, dissolving strip, films, or semi-solid formulation) and an effective amount of the solid formulation is about 0.5 mg, about 1 mg, about 10 mg, about 25 mg, about 50 mg, about 100 mg, about 250 mg, about 500 mg, about 750 mg, about 1 g, about 2 g, about 5 g, about 10 g, or more. In some embodiments, an effective amount of the solid formulation is between about 0.5 mg and about 100 mg. In some embodiments, an effective amount of the solid formulation is between about 100 mg and about 500 mg. In some embodiments, an effective amount of the solid formulation is between about 500 mg and about 1000 mg.

In some embodiments, the ion channel activator (e.g., a TRPV1 channel activator, a TRPA1 channel activator, an ASIC channel activator, or combination thereof) is ingested by the subject, e.g., is swallowed by the subject. In some embodiments, the ion channel activator (e.g., a TRPV1 channel activator, a TRPA1 channel activator, an ASIC channel activator, or combination thereof) is held in the mouth by the subject, e.g., is not swallowed by the subject. In some embodiments, the holding in the mouth may further comprise e.g., actively swirling the ion channel activator in the mouth of the subject, or placing the against ion channel activator on the skin or surface of the mouth or tongue (e.g., sublingual delivery). In some embodiments, the ion channel activator (e.g., a TRPV1 channel activator, a TRPA1 channel activator, an ASIC channel activator, or combination thereof) is dissolved in the mouth of the subject or chewed by the subject prior to swallowing.

In another aspect, the invention features a method of treating a painful muscle contraction in a subject in need thereof, said method comprising orally administering to said subject a composition comprising an effective amount of an ion channel activator (e.g., a TRPV1 channel activator, a TRPA1 channel activator, an ASIC channel activator, or combination thereof) and a pharmaceutically acceptable excipient. In some embodiments, said method comprises orally administering to a subject a composition comprising a plurality of (e.g., two or three) ion channel activators (e.g., TRPV1 channel activators, TRPA1 channel activators, ASIC channel activators, or combinations thereof) and a pharmaceutically acceptable excipient. In some embodiments, said method comprises orally administering to a subject a composition comprising an ion channel activator (e.g., a TRPV1 channel activator, a TRPA1 channel activator, an ASIC channel activator, or combination thereof) and a plurality of pharmaceutically acceptable excipients. In some embodiments, said painful muscle contraction is a muscle contraction of the head or neck. In some embodiments, said painful muscle contraction is associated with tension headache, cluster headache, or migraine headache.

In another aspect, the invention features a method of treating tactile sensitivity in a subject in need thereof, said method comprising orally administering to said subject a composition comprising an effective amount of an ion channel activator (e.g., a TRPV1 channel activator, a TRPA1 channel activator, an ASIC channel activator, or combination thereof) and a pharmaceutically acceptable excipient. In some embodiments, said method comprises orally administering to a subject a composition comprising a plurality of (e.g., two or three) ion channel activators (e.g., TRPV1 channel activators, TRPA1 channel activators, ASIC channel activators, or combinations thereof) and a pharmaceutically acceptable excipient. In some embodiments, said method comprises orally administering to a subject a composition comprising an ion channel activator (e.g., a TRPV1 channel activator, a TRPA1 channel activator, an ASIC channel activator, or combination thereof) and a plurality of pharmaceutically acceptable excipients. In some embodiments, said tactile sensitivity is associated with autism, dyspraxia, neuralgia, anxiety disorders, venomous bites, or venomous stings. In some embodiments, said anxiety disorder is selected from the group consisting of panic disorder, obsessive-compulsive disorder (OCD), post-traumatic stress disorder (PTSD), social anxiety disorder, phobia, and generalized anxiety disorder (GAD).

In another aspect, the invention features a method of treating a dystonia in a subject in need thereof, said method comprising orally administering to said subject a composition comprising an effective amount of an ion channel activator (e.g., a TRPV1 channel activator, a TRPA1 channel activator, an ASIC channel activator, or combination thereof) and a pharmaceutically acceptable excipient. In some embodiments, said method comprises orally administering to a subject a composition comprising a plurality of (e.g., two or three) ion channel activators (e.g., TRPV1 channel activators, TRPA1 channel activators, ASIC channel activator, or combinations thereof) and a pharmaceutically acceptable excipient. In some embodiments, said method comprises orally administering to a subject a composition comprising an ion channel activator (e.g., a TRPV1 channel activator, a TRPA1 channel activator, an ASIC channel activator, or combination thereof) and a plurality of pharmaceutically acceptable excipients. In some embodiments, said dystonia is selected from the group consisting of: focal dystonia, blepharospasm, cervical dystonia, cranial dystonia, laryngeal dystonia, back spasms, hand dystonia, or leg cramps due to spinal stenosis.

In another aspect, the invention features a method of treating a peripheral nervous system (PNS) condition in a subject in need thereof, said method comprising orally administering to said subject a composition comprising an effective amount of an ion channel activator (e.g., a TRPV1 channel activator, a TRPA1 channel activator, an ASIC channel activator, or combination thereof) and a pharmaceutically acceptable excipient to said subject. In some embodiments, said method comprises orally administering to a subject a composition comprising a plurality of (e.g., two or three) ion channel activators (e.g., TRPV1 channel activators, TRPA1 channel activators, ASIC channel activators, or combinations thereof) and a pharmaceutically acceptable excipient. In some embodiments, said method comprises orally administering to a subject a composition comprising an ion channel activator (e.g., a TRPV1 channel activator, a TRPA1 channel activator, an ASIC channel activator, or combination thereof) and a plurality of pharmaceutically acceptable excipients. In some embodiments, said PNS condition is selected from the group consisting of: cramp fasciculation syndrome, Isaacs' Syndrome or neuromyotonia (NMT), peripheral neuropathy, carpal tunnel syndrome, and Epstein-Barr virus (EBV) infection.

In another aspect, the invention features a method of treating a throat condition in a subject in need thereof, said method comprising orally administering to said subject a composition comprising an effective amount of an ion channel activator (e.g., a TRPV1 channel activator, a TRPA1 channel activator, an ASIC channel activator, or combination thereof) and a pharmaceutically acceptable excipient. In some embodiments, said method comprises orally administering to a subject a composition comprising a plurality of (e.g., two or three) ion channel activators (e.g., TRPV1 channel activators, TRPA1 channel activators, ASIC channel activators, or combinations thereof) and a pharmaceutically acceptable excipient. In some embodiments, said method comprises orally administering to a subject a composition comprising an ion channel activator (e.g., a TRPV1 channel activator, a TRPA1 channel activator, an ASIC channel activator, or combination thereof) and a plurality of pharmaceutically acceptable excipients. In some embodiments, said throat condition is associated with chemical injury, cancer, surgical injury, or pathogen infection. In some embodiments, said throat condition is selected from the group consisting of: acid reflux, laryngospasm, dysphagia, and spasmodic dysphonias.

In another aspect, the invention features a method of treating a condition associated with an electrolyte imbalance or vitamin deficiency in a subject in need thereof, said method comprising orally administering to said subject a composition comprising an effective amount of an ion channel activator (e.g., a TRPV1 channel activator, a TRPA1 channel activator, an ASIC channel activator, or combination thereof) and a pharmaceutically acceptable excipient. In some embodiments, said method comprises orally administering to a subject a composition comprising a plurality of (e.g., two or three) ion channel activators (e.g., TRPV1 channel activators, TRPA1 channel activators, ASIC channel activators, or combinations thereof) and a pharmaceutically acceptable excipient. In some embodiments, said method comprises orally administering to a subject a composition comprising an ion channel activator (e.g., a TRPV1 channel activator, a TRPA1 channel activator, an ASIC channel activator, or combination thereof) and a plurality of pharmaceutically acceptable excipients. In some embodiments, said condition is selected from the group consisting of: hyponatremia, kidney disease, rickets, calcium, magnesium deficiency, thiamine deficiency, hypoparathyroidism, medullary cystic disease, and adrenocortical carcinoma.

In another aspect, the invention features a method of treating a central nervous system (CNS) condition in a subject in need thereof, said method comprising orally administering to said subject a composition comprising an effective amount of an ion channel activator (e.g., a TRPV1 channel activator, a TRPA1 channel activator, an ASIC channel activator, or combination thereof) and a pharmaceutically acceptable excipient. In some embodiments, said method comprises orally administering to a subject a composition comprising a plurality of (e.g., two or three) ion channel activators (e.g., TRPV1 channel activators, TRPA1 channel activators, ASIC channel activators, or combination thereof) and a pharmaceutically acceptable excipient. In some embodiments, said method comprises orally administering to a subject a composition comprising an ion channel activator (e.g., a TRPV1 channel activator, a TRPA1 channel activator, an ASIC channel activator, or combination thereof) and a plurality of pharmaceutically acceptable excipients. In some embodiments, said CNS condition is associated with a tumor. In some embodiments, said CNS condition is selected from the group consisting of: multiple sclerosis, amyotrophic lateral sclerosis, cerebral palsy, stroke, motor neuron disease, spinal injury, and stenosis.

In another aspect, the invention features a method of treating a muscle condition or disorder in a subject in need thereof, said method comprising orally administering to said subject a composition comprising an effective amount of an ion channel activator (e.g., a TRPV1 channel activator, a TRPA1 channel activator, an ASIC channel activator, or combination thereof) and a pharmaceutically acceptable excipient. In some embodiments, said method comprises orally administering to a subject a composition comprising a plurality of (e.g., two or three) ion channel activators (e.g., TRPV1 channel activators, TRPA1 channel activators, ASIC channel activators, or combinations thereof) and a pharmaceutically acceptable excipient. In some embodiments, said method comprises orally administering to a subject a composition comprising an ion channel activator (e.g., a TRPV1 channel activator, a TRPA1 channel activator, an ASIC channel activator, or combination thereof) and a plurality of pharmaceutically acceptable excipients. In some embodiments, said muscle condition is associated with muscle pain, muscle spasms, muscle cramps, fasciculations, or any combination thereof. In some embodiments, the muscle condition or disorder is a neuromuscular disorder (e.g., multiple sclerosis, spinal cord spasticity, spinal muscle atrophy, myasthenia gravis, spinal cord injury, traumatic brain injury, cerebral palsy, hereditary spastic paraplegia, motor neuron disease (e.g., amyotrophic lateral sclerosis, primary lateral sclerosis, progressive muscular atrophy, progressive bulbar palsy, pseudobulbar palsy, spinal muscular atrophy, progressive spinobulbar muscular atrophy (e.g., Kennedy's disease), or post-polio syndrome), neuralgia, fibromyalgia, Machado-Joseph disease, cramp fasciculation syndrome, carpal tunnel syndrome, acrodynia, neurofibromatosis, neuromyotonias (e.g., focal neuromyotonia, Isaacs' syndrome), peripheral neuropathy, piriformis syndrome, plexopathy (e.g., Brachial plexopathy or Lumbosacral plexobathy), radiculopathy (e.g., lower lumbar radiculopathy), and encephalitis).

In some embodiments, said muscle condition is muscle pain, muscle spasms, muscle cramps, spasticity, or fasciculations associated with motor neuron disease (e.g., amyotrophic lateral sclerosis, primary lateral sclerosis, progressive muscular atrophy, progressive bulbar palsy, pseudobulbar palsy, spinal muscular atrophy, progressive spinobulbar muscular atrophy (e.g., Kennedy's disease), or post-polio syndrome).

In some embodiments, said muscle condition is associated with treatment of said subject with dialysis, diuretics, β-blockers, statins, fibrates, β2-agonists, ACE inhibitors, ARBs, anti-psychotic medications, or any combination thereof. In some embodiments, said muscle condition is associated with treatment of said subject with statins and fibrates. In some embodiments, said muscle condition occurs in one or more skeletal muscles. In some embodiments, said muscle condition is refractory to an approved treatment. In some embodiments, said approved treatment is botox, cyclopenzaprine, orphenadrine, baclofen, or any combination thereof. In some embodiments, said muscle condition is fibromyalgia. In some embodiments, said muscle condition involves muscle claudication pain. In some embodiments, said muscle claudication pain is associated with inactivity, restriction, economy class syndrome, paralysis, peripheral artery disease, or immobilization.

In another aspect, the invention features a method of treating a respiratory condition in a subject in need thereof, said method comprising orally administering to said subject a composition comprising an effective amount of an ion channel activator (e.g., a TRPV1 channel activator, a TRPA1 channel activator, an ASIC channel activator, or combination thereof) and a pharmaceutically acceptable excipient. In some embodiments, said method comprises orally administering to a subject a composition comprising a plurality of (e.g., two or three) ion channel activators (e.g., TRPV1 channel activators, TRPA1 channel activators, ASIC channel activators, or combination thereof) and a pharmaceutically acceptable excipient. In some embodiments, said method comprises orally administering to a subject a composition comprising an ion channel activator (e.g., a TRPV1 channel activator, a TRPA1 channel activator, an ASIC channel activator, or combination thereof) and a plurality of pharmaceutically acceptable excipients. In some embodiments, said respiratory condition comprises asthma, chronic obstructive pulmonary disease, bronchitis, emphysema, pneumonia, cystic fibrosis, influenza, or a cold.

In another aspect, the invention features a method of treating a cough in a subject in need thereof, said method comprising orally administering to said subject a composition comprising an effective amount of an ion channel activator (e.g., a TRPV1 channel activator, a TRPA1 channel activator, an ASIC channel activator, or combination thereof), and a pharmaceutically acceptable excipient. In some embodiments, said method comprises orally administering to a subject a composition comprising a plurality of (e.g., two or three) ion channel activators (e.g., TRPV1 channel activators, TRPA1 channel activators, ASIC channel activators, or combination thereof), and a pharmaceutically acceptable excipient. In some embodiments, said method comprises orally administering to a subject a composition comprising an ion channel activator (e.g., a TRPV1 channel activator, a TRPA1 channel activator, an ASIC channel activator, or combination thereof), and a plurality of pharmaceutically acceptable excipients. In some embodiments, said cough is associated with a respiratory condition (e.g., asthma, chronic obstructive pulmonary disease, bronchitis, emphysema, pneumonia, cystic fibrosis, influenza, or a cold), exposure to an allergen, or inflammation.

In another aspect, the invention features a method of treating sarcoidosis in a subject in need thereof, said method comprising orally administering to said subject a composition comprising an effective amount an ion channel activator (e.g., a TRPV1 channel activator, a TRPA1 channel activator, an ASIC channel activator, or combination thereof), and a pharmaceutically acceptable excipient. In some embodiments, said method comprises orally administering to a subject a composition comprising a plurality of (e.g., two or three) ion channel activators (e.g., TRPV1 channel activators, TRPA1 channel activators, ASIC channel activators, or combinations thereof), and a pharmaceutically acceptable excipient. In some embodiments, said method comprises orally administering to a subject a composition comprising an ion channel activator (e.g., a TRPV1 channel activator, a TRPA1 channel activator, an ASIC channel activator, or combination thereof), and a plurality of pharmaceutically acceptable excipients.

In another aspect, the invention features a method of treating a connective tissue disease in a subject in need thereof, said method comprising orally administering to said subject a composition comprising an effective amount an ion channel activator (e.g., a TRPV1 channel activator, a TRPA1 channel activator, an ASIC channel activator, or combination thereof), and a pharmaceutically acceptable excipient. In some embodiments, said method comprises orally administering to a subject a composition comprising a plurality of (e.g., two or three) ion channel activators (e.g., TRPV1 channel activators, TRPA1 channel activators, ASIC channel activators, or combinations thereof), and a pharmaceutically acceptable excipient. In some embodiments, said method comprises orally administering to a subject a composition comprising an ion channel activator (e.g., a TRPV1 channel activator, a TRPA1 channel activator, an ASIC channel activator, or combination thereof), and a plurality of pharmaceutically acceptable excipients. In some embodiments, said connective tissue disease is selected from the group consisting of Ehlers-Danlos syndrome, epidermolysis bullosa, Marfan syndrome, osteogenesis imperfect, arthritis, scleroderma, Sjögren's syndrome, lupus, vasculitis, mixed connective tissue disease, cellulitis, polymyositis, and dermatomyositis. In some embodiments, said arthritis is rheumatoid arthritis, osteoarthritis, gout, or psioratic arthritis, or wherein said vasculitis is Wegener's granulomatosis or Churg-Strauss Syndrome.

In any of the aspects described above, in some embodiments, the method of treatment comprises orally administering to a subject a composition comprising an ion channel activator (e.g., a TRPV1 channel activator, a TRPA1 channel activator, an ASIC channel activator, or combination thereof), wherein said TRPV1 channel activator is capsaicin, a capsaicinoid, or a capsinoid, or is selected from the group consisting of oleoylethanolamide, N-oleoyldopamine, 3-methyl-N-oleoyldopamine, oleamide, capsiate, a 1-monoacylglycerol having C18 and C20 unsaturated and C8-C12 saturated fatty acid, a 2-monoacylglycerol having C18 and C20 unsaturated fatty acids, miogadial, miogatrial, polygodial, a terpenoid with an alpha,beta-unsaturated 1,4-dialdehyde moiety, sanshool, evodiamine, acesulfame-K, cyclamate, CuSO₄, ZnSO₄, FeSO₄, arvanil, anandamide, N-arachidonoyl-dopamine, flufenamic acid dopamide, a dopamine amide of fenamic acid, 4-hydroxynonenal, or 1-[2-(1-adamantyl)ethyl]-1-pentyl-3-[3-(4-pyridyl)propyl]urea, gingerol, and salts of magnesium.

In particular embodiments, the capsaicinoid is capsaicin. In some aspects of this embodiment, the TRPV1 channel activator is present from about 0.001% to about 10% (w/w) or from about 0.001% to about 10% (v/v).

In some embodiments, the TRPV1 channel activator is naturally occurring or non-naturally occurring. In some embodiments, said naturally occurring TRPV1 channel activator is selected from the group consisting of: capsaicin, dihydrocapsaicin, nordihydrocapsaicin, homodihydrocapsaicin, homocapsaicin, nonivamide, pseudocapsaicin, resiniferatoxin, tinyatoxin, capsiate, dihydrocapsiate, nordihydrocapsiate, norcapsaicin, capsiconiate, dihydrocapsiconiate and other coniferyl esters, capsiconinoid, and 3-hydroxyacetanilide.

In some embodiments, the non-naturally occurring TRPV1 channel is selected from the group consisting of: 4-[((6E)-8-methylnon-6-enoylamino)methyl]-2-methyoxyphenyl formate, 4-[((6E)-8-methylnon-6-enoylamino)methyl]-2-methyoxyphenyl acetate, 4-[((6E)-8-methylnon-6-enoylamino)methyl]-2-methyoxyphenyl propanoate, 4-[((6E)-8-methylnon-6-enoylamino)methyl]-2-methyoxyphenyl butanoate, 4-[((6E)-8-methylnon-6-enoylamino)methyl]-2-methoxyphenyl 2,2-dimethylpropanoate, 4-[((6E)-8-methylnon-6-enoylamino)methyl]-2-methyoxyphenyl octadecanoate, 4-[((6E)-8-methylnon-6-enoylamino)methyl]-2-methyoxyphenyl{4-[((6E)-8-methylnon-6-enoylamino)methyl]-2-methoxyphenoxy}formate, homovanillyl 8-methylnonanoate, 3-(3-methoxy-4-hydroxyphenyl)propyl 8-methylnonanoate, 8-methylnonyl homovanillate, 8-methylnonanoic acid-substituted benzyl ester derivative, heptanoyl isobutylamide, heptanoyl guaiacylamide, 7-phenylhept-6-yne-acid-4-hydroxy-3-methoxybenzylamide, dohevanil, denatonium capsaicinate, N-[4-(2-aminoethoxy)-3-methoxybenzyl]-N′-[2-(4-chlorophenyl)ethyl]thiourea, N-[4-(2-aminoethoxy)-3-methoxybenzyl]-N′-[2-(4-fluorophenyl)ethyl]thiourea, N-[4-(2-aminoethoxy)-3-methoxybenzyl]-N′-[2-(2,4-dichlorophenyl)ethyl]thiourea, N-[4-(2-aminoethoxy)-3-methoxybenzyl]-N′-[2-(4-benzyloxyphenyl)ethyl]thiourea, N-[4-(2-aminoethoxy)-3-methoxybenzyl]-N′-[2-(4-(N-octyloxy)phenyl)ethyl]thiourea, N-[4-(2-aminoethoxy)-3-methoxybenzyl]-N′-[4-N-octyloxybenzyl]thiourea, N-phenylmethylalkynamide capsaicin derivatives, N-(4-O-glycerol-3-methoxybenzyl)nonamide, N-nonanoyl vanillylamide-4-glyceryl ether, N-(4-O-acetic acid sodium)-3-methoxybenzyl-nonamide (sodium N-nonanoyl vanillylamide-4-O-acetate), N-(4-O-glycol-3-methoxybenzyl)-nonamide (N-nonanoyl vanillylamide-4-glycol ether), 20-homovanillyl-mezerein, 20-homovanillyl-12-deoxyphorbol-13-phenylacetate), civamide (N-[(4-hydroxy-3-methoxyphenyl)-methyl]-8-methyl-(Z)-6-nonemamide), nuvanil, capsavanil, olvanil, arvanil, and palvanil (N-palmitoyl-vanillamide).

In some embodiments of the invention, the TRPA1 channel activator is allyl isothiocyanate, a gingerol, cinnamaldehyde, acrolein, farnesyl thiosalicylic acid, Δ₉-tetrahydrocannabinol, eugenol, a shogaol, a sanshool, allicin, diallyl sulfide, diallyl disulfide, diallyl trisulfide, or farnesyl thioacetic acid. In some aspects of this embodiment, the TRPA1 channel activator is present from about 0.001% to about 10% (w/w) or from about 0.001% to about 10% (v/v).

In some embodiments, the ASIC channel activator comprises acetic acid, phosphoric acid, citric acid, malic acid, succinic acid, lactic acid, tartaric acid, fumaric acid, or ascorbic acid. In some embodiments, the ASIC channel activator is present from about 0.001% to about 10% (w/w) or from about 0.001% to about 10% (v/v).

In some embodiments, the composition is formulated as a liquid or a solid. In some embodiments, the composition is formulated as a liquid. In some embodiments, the liquid is selected from the group consisting of emulsions, microemulsions, solutions, suspensions, syrups (e.g., syrup concentrates), linctuses, drops, sprays, and elixirs. In some embodiments, the composition is formulated as a solid. In some embodiments, the solid is selected from the group consisting of tablets, capsules, powders, crystals, pastes, gels, lozenges (e.g., liquid filled lozenges), gums, candies, chews, foodstuffs, dissolving strips, films, and semi-solid formulations. In some embodiments, said solid is a tablet or capsule. In some embodiments, said capsule is a hard or soft capsule.

In another aspect, the invention features a method of treating a subject for unwanted or abnormal muscle contraction (e.g., cramp, spasm, dystonia, or fasciculation) or absence of a normal muscle contraction (e.g., gait abnormalities, e.g., foot drop) comprising: acquiring, e.g., directly or indirectly, knowledge of a result of a test for the efficacy of the administration of a test aliquot of a composition comprising an ion channel activator (e.g., a TRPV1 channel activator, a TRPA1 channel activator, an ASIC channel activator, or combination thereof) for alleviation of test muscle contraction in said subject; and administering, e.g., in response to said result, an amount of a composition comprising an ion channel activator (e.g., a TRPV1 channel activator, a TRPA1 channel activator, an ASIC channel activator, or combination thereof) sufficient to alleviate unwanted or abnormal muscle contraction or absence of normal muscle contraction to said subject. In some embodiments, the test comprises directly acquiring said knowledge. In some embodiments, the test comprises further performing said test.

In some embodiments, said muscle contraction comprises a muscle cramp. In some embodiments, said muscle contraction comprises a muscle spasm. In some embodiments, said muscle contraction comprises a dystonia. In some embodiments, said muscle contraction comprises a fasciculation. In some embodiments, said muscle contraction occurs in a skeletal muscle. In some embodiments, said muscle contraction occurs in a smooth muscle. In some embodiments, the test muscle contraction is a test muscle cramp or a test muscle spasm.

In some embodiments, said composition comprises a plurality of (e.g., two or three) ion channel activators (e.g., TRPV1 channel activators, TRPA1 channel activators, ASIC channel activators, or combinations thereof). In some embodiments, said composition further comprises a pharmaceutically acceptable excipient. In some embodiments, said composition comprises an ion channel activator (e.g., a TRPV1 channel activator, a TRPA1 channel activator, an ASIC channel activator, or combination thereof), and a plurality of pharmaceutically acceptable excipients.

In some embodiments, the TRPV1 channel activator is a capsaicinoid, a capsinoid, oleoylethanolamide, N-oleoyldopamine, 3-methyl-N-oleoyldopamine, oleamide, capsiate, a 1-monoacylglycerol having C18 and C20 unsaturated and C8-C12 saturated fatty acid, a 2-monoacylglycerol having C18 and C20 unsaturated fatty acids, miogadial, miogatrial, polygodial, a terpenoid with an alpha,beta-unsaturated 1,4-dialdehyde moiety, sanshool, evodiamine, acesulfame-K, cyclamate, CuSO₄, ZnSO₄, FeSO₄, arvanil, anandamide, N-arachidonoyl-dopamine, flufenamic acid dopamide, a dopamine amide of fenamic acid, 4-hydroxynonenal, 1-[2-(1-adamantyl)ethyl]-1-pentyl-3-[3-(4-pyridyl)propyl]urea, or gingerol.

In particular embodiments, the capsaicinoid is capsaicin. In some aspects of this embodiment, the TRPV1 channel activator is present from about 0.001% to about 10% (w/w) or from about 0.001% to about 10% (v/v).

In some embodiments, the TRPV1 channel activator is naturally occurring or non-naturally occurring. In some embodiments, said naturally occurring TRPV1 channel activator is selected from the group consisting of: capsaicin, dihydrocapsaicin, nordihydrocapsaicin, homodihydrocapsaicin, homocapsaicin, nonivamide, pseudocapsaicin, resiniferatoxin, tinyatoxin, capsiate, dihydrocapsiate, nordihydrocapsiate, norcapsaicin, capsiconiate, dihydrocapsiconiate and other coniferyl esters, capsiconinoid, and 3-hydroxyacetanilide.

In some embodiments, the non-naturally occurring TRPV1 channel is selected from the group consisting of: 4-[((6E)-8-methylnon-6-enoylamino)methyl]-2-methyoxyphenyl formate, 4-[((6E)-8-methylnon-6-enoylamino)methyl]-2-methyoxyphenyl acetate, 4-[((6E)-8-methylnon-6-enoylamino)methyl]-2-methyoxyphenyl propanoate, 4-[((6E)-8-methylnon-6-enoylamino)methyl]-2-methyoxyphenyl butanoate, 4-[((6E)-8-methylnon-6-enoylamino)methyl]-2-methoxyphenyl 2,2-dimethylpropanoate, 4-[((6E)-8-methylnon-6-enoylamino)methyl]-2-methyoxyphenyl octadecanoate, 4-[((6E)-8-methylnon-6-enoylamino)methyl]-2-methyoxyphenyl{4-[((6E)-8-methylnon-6-enoylamino)methyl]-2-methoxyphenoxy}formate, homovanillyl 8-methylnonanoate, 3-(3-methoxy-4-hydroxyphenyl)propyl 8-methylnonanoate, 8-methylnonyl homovanillate, 8-methylnonanoic acid-substituted benzyl ester derivative, heptanoyl isobutylamide, heptanoyl guaiacylamide, 7-phenylhept-6-yne-acid-4-hydroxy-3-methoxybenzylamide, dohevanil, denatonium capsaicinate, N-[4-(2-aminoethoxy)-3-methoxybenzyl]-N′-[2-(4-chlorophenyl)ethyl]thiourea, N-[4-(2-aminoethoxy)-3-methoxybenzyl]-N′-[2-(4-fluorophenyl)ethyl]thiourea, N-[4-(2-aminoethoxy)-3-methoxybenzyl]-N′-[2-(2,4-dichlorophenyl)ethyl]thiourea, N-[4-(2-aminoethoxy)-3-methoxybenzyl]-N′-[2-(4-benzyloxyphenyl)ethyl]thiourea, N-[4-(2-aminoethoxy)-3-methoxybenzyl]-N′-[2-(4-(N-octyloxy)phenyl)ethyl]thiourea, N-[4-(2-aminoethoxy)-3-methoxybenzyl]-N′-[4-N-octyloxybenzyl]thiourea, N-phenylmethylalkynamide capsaicin derivatives, N-(4-O-glycerol-3-methoxybenzyl)nonamide, N-nonanoyl vanillylamide-4-glyceryl ether, N-(4-O-acetic acid sodium)-3-methoxybenzyl-nonamide (sodium N-nonanoyl vanillylamide-4-O-acetate), N-(4-o-glycol-3-methoxybenzyl)-nonamide (N-nonanoyl vanillylamide-4-glycol ether), 20-homovanillyl-mezerein, 20-homovanillyl-12-deoxyphorbol-13-phenylacetate), civamide (N-[(4-hydroxy-3-methoxyphenyl)-methyl]-8-methyl-(Z)-6-nonemamide), nuvanil, capsavanil, olvanil, arvanil, and palvanil (N-palmitoyl-vanillamide).

In some embodiments of the invention, the TRPA1 channel activator is allyl isothiocyanate, a gingerol, cinnamaldehyde, acrolein, farnesyl thiosalicylic acid, Δ₉-tetrahydrocannabinol, eugenol, a shogaol, a sanshool, allicin, diallyl sulfide, diallyl disulfide, diallyl trisulfide, or farnesyl thioacetic acid. In some aspects of this embodiment, the TRPA1 channel activator is present from about 0.001% to about 10% (w/w) or from about 0.001% to about 10% (v/v).

In some embodiments, the ASIC channel activator comprises acetic acid, phosphoric acid, citric acid, malic acid, succinic acid, lactic acid, tartaric acid, fumaric acid, or ascorbic acid. In some embodiments, the ASIC channel activator is present from about 0.001% to about 10% (w/w) or from about 0.001% to about 10% (v/v).

In some embodiments, the subject has a central nervous system disorder or injury, e.g., a brain injury, stroke, or traumatic spinal cord injury. In some embodiments, the subject has been diagnosed with or identified as having multiple sclerosis. In some embodiments, the subject has been diagnosed with or identified as having dystonia, e.g., cervical dystonia. In some embodiments, the subject has been diagnosed with or identified as having spinal cord spasticity. In some embodiments, said subject has been diagnosed with or identified as having a disorder associated with muscle cramps, e.g., any of the disorders disclosed herein, e.g., night cramps, multiple sclerosis, spinal cord spasticity, or dystonia.

In some embodiments, said muscle contraction being selected, treated, or diagnosed, comprises a contraction in a muscle other than a muscle that is contracted in the test muscle contraction. In some embodiments, said test muscle contraction comprises a contraction in a muscle of the foot, e.g., the flexor hallucis brevis muscle, and the muscle cramp comprises a cramp in a muscle other than the foot, e.g., the flexor hallucis brevis muscle. In some embodiments, said muscle contraction is not induced by applied electrical stimulation.

In some embodiments, said muscle contraction is a night cramp. In some embodiments, said muscle contraction is associated with multiple sclerosis. In some embodiments, said muscle contraction is associated with spinal cord spasticity. In some embodiments, said muscle contraction is associated with dystonia.

In some embodiments, said test comprises inducing said test muscle cramp by application of electrical stimulation, e.g., percutaneous stimulation or surface stimulation. In some embodiments, said test comprises determining that a muscle contraction can be induced in a subject by application of electrical stimulation, e.g., percutaneous stimulation or surface stimulation.

In some embodiments, said test comprises: a) administering the test aliquot of the composition to said subject; b) inducing, e.g., by application of electrical stimulation, e.g., percutaneous stimulation or surface stimulation, a test muscle contraction; and c) evaluating the effect of administering the test aliquot of the composition on test muscle contraction. In some embodiments, step a is performed before step b. In some embodiments, step a is performed after step b.

In some embodiments, said test comprises: a) administering the test aliquot of the composition to said subject; b) inducing, e.g., by application of electrical stimulation, e.g., percutaneous stimulation or surface stimulation, a test muscle contraction; and c) evaluating the effect of administering the composition on test muscle contraction, e.g., by evaluating the electrical activity of said test muscle, e.g., by EMG.

In some embodiments, said test comprises: a) inducing, e.g., by application of electrical stimulation, e.g., percutaneous stimulation or surface stimulation, a first test muscle contraction; b) administering the test aliquot of the composition to said subject; c) inducing, e.g., by application of electrical stimulation, e.g., percutaneous stimulation or surface stimulation, a second test muscle contraction; and d) evaluating the effect of administering the composition on said second test muscle contraction. In some embodiments, step b is performed before step c. In some embodiments, step c is performed before step b. In some embodiments, the steps are performed in the order of a, b, c, and d.

In some embodiments, said test further comprises: e) providing a value, e.g., a reference value, e.g. reference profile, for a muscle contraction parameter, e.g., a value for intensity or duration of, said first test muscle contraction, e.g., by evaluating the electrical activity of said test muscle, e.g., by EMG; and optionally, f) providing a value, e.g., a treatment value, e.g., a treatment profile, for a muscle contraction parameter, e.g., a value for intensity or duration of, said second test muscle contraction, e.g., by evaluating the electrical activity of said test muscle, e.g., by EMG. In some embodiments, the test comprises comparing the value from step e with the value from step f to evaluate the effectiveness of a test aliquot of the composition on test muscle contraction. In some embodiments, said muscle contraction parameter is the area under the curve, the peak amplitude, or the duration of the test muscle contraction. In some embodiments, a decrease in the value from step f compared to the value from step e is indicative of efficacy in alleviating said test muscle cramp. In some embodiments, a decrease by a preselected amount, e.g., a decrease of at least about 5%, 10%, 15%, 20%, 25%, 30%, 35% or 50%, is indicative of efficacy in alleviating said test muscle contraction.

In some embodiments, said test comprises: a) inducing, e.g., by application of electrical stimulation, e.g., percutaneous stimulation or surface stimulation, a first test muscle contraction; b) administering the test aliquot of the composition to said subject; c) inducing, e.g., by application of electrical stimulation, e.g., percutaneous stimulation or surface stimulation, a second test muscle contraction; d) evaluating the effect of administering the composition on test contraction cramping; e) providing a value for a muscle contraction parameter, e.g., a value for intensity or duration of, said first test muscle contraction, e.g., by evaluating the electrical activity of said test muscle, e.g., by EMG; and f) providing a value for a muscle contraction parameter, e.g., a value for intensity or duration of, said second test muscle contraction, e.g., by evaluating the electrical activity of said test muscle, e.g., by EMG. In some embodiments, step b is performed before step c. In some embodiments, steps b and c are performed within a preselected time of one another, e.g., they are performed sufficiently close in time that step b will modulate step c. In some embodiments, said test muscle contraction comprises a contraction in a muscle of the foot, e.g., the flexor hallucis brevis muscle. In some embodiments, a decrease in the value from step f compared to the value from step e is indicative of efficacy in alleviating said test muscle contraction.

In some embodiments, said test comprises: a) applying a first electrical stimulus to a test muscle of the subject to induce a test muscle contraction; b) measuring the electrical activity of said test muscle to provide a reference profile, e.g., by EMG; c) administering a test aliquot of the composition; d) applying a second electrical stimulus to the test muscle of the subject after a preselected period of time after administration of the test aliquot of the composition; e) measuring the electrical activity of said test muscle to generate a treatment profile; f) comparing the treatment profile to the reference profile to determine reduction or prevention of the test muscle contraction after administration of the test aliquot. In some embodiments, the period of time between step c and step d is at least about 10 minutes, 15 minutes, 30 minutes, 1 hour, etc.

In some embodiments, comprising determining the area under the curve from the reference profile and the treatment profile, wherein when the area under the curve from the treatment profile is decreased compared to the reference profile, the test muscle contraction is reduced or prevented.

In some embodiments, the test further comprises: a) determining the threshold frequency for inducing a first test muscle contraction; b) administering a test aliquot of a composition comprising an ion channel activator (e.g., TRPV1 channel activator, TRPA1 channel activator, ASIC channel activator, or combination thereof); c) determining the threshold frequency for inducing a second test muscle contraction; d) comparing the threshold frequency to evaluate the effectiveness of a test aliquot of the composition on test muscle contraction.

In some embodiments, said test aliquot of the composition comprises a plurality of (e.g., two or three) ion channel activators (e.g., TRPV1 channel activators, TRPA1 channel activators, ASIC channel activators, or combinations thereof). In some embodiments, said test aliquot of the composition further comprises a pharmaceutically acceptable excipient. In some embodiments, said test aliquot of the composition comprises an ion channel activator (e.g., a TRPV1 channel activator, a TRPA1 channel activator, an ASIC channel activator, or combination thereof), and a plurality of pharmaceutically acceptable excipients.

In some embodiments, said test aliquot of the composition comprises a TRP channel activator or an ASIC channel activator. In some embodiments, said TRP channel activator comprises a TRPV1 activator and a TRPA1 activator. In some embodiments, said composition comprises a TRP activator and an ASIC channel activator. In certain embodiments, the TRPV1 agonist is a capsaicinoid, e.g., capsaicin. In some embodiments, the TRPV1 channel activated by said ion channel activator (e.g., a TRPV1 channel activator, TRPA1 channel activator, ASIC channel activator or combination thereof) is present on a sensory neuron in the mouth, the esophagus and/or the stomach. In some embodiments, said ion channel activator (e.g., a TRPV1 channel activator, TRPA1 channel activator, ASIC channel activator or combination thereof) increases inhibitory signaling to alpha motor neurons.

In some embodiments, the composition is formulated as a liquid or a solid. In some embodiments, the liquid is selected from the group consisting of emulsions, microemulsions, solutions, suspensions, syrups (e.g., syrup concentrates), linctuses, drops, sprays, and elixirs. In some embodiments, the composition is formulated as a solid. In some embodiments, the solid is selected from the group consisting of tablets, capsules, powders, crystals, pastes, gels, lozenges (e.g., liquid filled lozenges), gums, candies, chews, foodstuffs, dissolving strips, films, and semi-solid formulations. In some embodiments, said solid is a tablet or capsule. In some embodiments, said capsule is a hard or soft capsule.

In another aspect, the invention features a method of evaluating a subject for abnormal or unwanted muscle contraction or absence of normal muscle contraction comprising: acquiring, e.g., indirectly or directly, knowledge of a result of a test for the efficacy of the administration of a test aliquot of a composition comprising an ion channel activator (e.g., a TRPV1 channel activator, a TRPA1 channel activator, an ASIC channel activator, or combination thereof) for alleviation of test muscle contraction in said subject; and responsive to said result, classifying said subject. In some embodiments, said subject has been diagnosed or identified as having a disorder associated with muscle cramps, e.g., any of the disorders disclosed herein, e.g., night cramps, multiple sclerosis, spinal cord spasticity, or dystonia. In some embodiments, the test comprises directly acquiring said knowledge. In some embodiments, the test further comprises performing said test.

In some embodiments, said result is indicative of a preselected level of alleviation of the test muscle contraction by administration of the test aliquot. In some embodiments, said result is indicative of the alleviation of the test muscle contraction by administration of the test aliquot.

In some embodiments, the test comprises classifying said subject as a candidate for treatment with a composition comprising an ion channel activator (e.g., a TRPV1 channel activator, a TRPA1 channel activator, an ASIC channel activator, or combination thereof). In some embodiments, said result is indicative of the failure to provide a preselected level of alleviation of the test muscle contraction by administration of the test aliquot. In some embodiments, said result is indicative of the absences of alleviation of the test muscle contraction by administration of the test aliquot. In some embodiments, the test comprises classifying said subject as not being a candidate for treatment with a composition comprising an ion channel activator (e.g., a TRPV1 channel activator, a TRPA1 channel activator, an ASIC channel activator, or combination thereof). In some embodiments, said method is computer implemented.

In some embodiments, said composition comprises a plurality of (e.g., two or three) ion channel activators (e.g., TRPV1 channel activators, TRPA1 channel activators, ASIC channel activators, or combinations thereof). In some embodiments, said composition further comprises a pharmaceutically acceptable excipient. In some embodiments, said composition comprises an ion channel activator (e.g., a TRPV1 channel activator, a TRPA1 channel activator, an ASIC channel activator, or combination thereof), and a plurality of pharmaceutically acceptable excipients.

In another aspect, the invention features a computer-implemented method of evaluating a subject for unwanted or abnormal muscle contraction, e.g., cramp, spasm, dystonia, or fasciculation or absence of a normal muscle contraction, e.g., gait abnormalities, e.g., foot drop comprising: a) acquiring, e.g., directly or indirectly, a value for a parameter related to the effect of administering a test aliquot of a composition comprising an ion channel activator (e.g., a TRPV1 channel activator, a TRPA1 channel activator, an ASIC channel activator, or combination thereof) on test muscle cramping, e.g., by evaluating the electrical activity of said test muscle, e.g., by EMG; b) evaluating the effectiveness of administering the test aliquot on a computer, e.g., by comparing a test value or profile with a treatment value or profile; c) responsive to the evaluation, comprising classifying said subject as a candidate for treatment with a composition comprising a capsaicinoid, capsinoid, or related analog or combination thereof.

In some embodiments, said composition comprises a plurality of (e.g., two or three) ion channel activators (e.g., TRPV1 channel activators, TRPA1 channel activators, ASIC channel activators, or combinations thereof). In some embodiments, said composition further comprises a pharmaceutically acceptable excipient. In some embodiments, said composition comprises an ion channel activator (e.g., a TRPV1 channel activator, a TRPA1 channel activator, an ASIC channel activator, or combination thereof), and a plurality of pharmaceutically acceptable excipients.

In some embodiments, the composition is formulated as a liquid or a solid. In some embodiments, the liquid is selected from the group consisting of emulsions, microemulsions, solutions, suspensions, syrups (e.g., syrup concentrates), linctuses, drops, sprays, and elixirs. In some embodiments, the composition is formulated as a solid. In some embodiments, the solid is selected from the group consisting of tablets, capsules, powders, crystals, pastes, gels, lozenges (e.g., liquid filled lozenges), gums, candies, chews, foodstuffs, dissolving strips, films, and semi-solid formulations. In some embodiments, said solid is a tablet or capsule. In some embodiments, said capsule is a hard or soft capsule.

In another aspect, the invention features a system comprising a memory; and a processing unit operative to: a) evaluate the effectiveness of administering a test aliquot of a composition comprising an ion channel activator (e.g., a TRPV1 channel activator, a TRPA1 channel activator, an ASIC channel activator, or combination thereof); b) responsive to the evaluation, comprising classifying said subject as a candidate for treatment with a composition comprising an ion channel activator (e.g., a TRPV1 channel activator, a TRPA1 channel activator, an ASIC channel activator, or combination thereof).

In another aspect, the invention features a computer-readable medium comprising computer-executable instructions that, when executed on a processor of a computer, perform a method comprising acts of: a) evaluating the effectiveness of administering a test aliquot of a composition comprising an ion channel activator (e.g., a TRPV1 channel activator, a TRPA1 channel activator, an ASIC channel activator, or combination thereof); b) responsive to the evaluation, comprising classifying said subject as a candidate for treatment with a composition comprising an ion channel activator (e.g., a TRPV1 channel activator, a TRPA1 channel activator, an ASIC channel activator, or combination thereof).

In some embodiments, said subject has been diagnosed or identified as having a disorder associated with muscle cramps, spasms, dystonia, or fasciculations, e.g., any of the disorders disclosed herein, e.g., night cramps, multiple sclerosis, spinal cord spasticity, or dystonia.

In another aspect, the invention features a kit comprising a liquid tight container comprising one or more of: one or a plurality of test aliquots of a composition comprising an ion channel activator (e.g., a TRPV1 channel activator, a TRPA1 channel activator, an ASIC channel activator, or combination thereof); one or a plurality of leads for conducting current to a subject and inducing a cramp; and one or a plurality of leads for measuring electrical activity associated with a cramp. In some embodiments, said kit further comprises a plurality, e.g., at least 2, 3, 4, 5, 6, 7, 8, 9, or 10, test aliquots of the composition.

In some embodiments, said test aliquot of the composition comprises a plurality of (e.g., two or three) ion channel activators (e.g., TRPV1 channel activators, TRPA1 channel activators, ASIC channel activators, or combinations thereof). In some embodiments, said test aliquot of the composition further comprises a pharmaceutically acceptable excipient. In some embodiments, said test aliquot of the composition comprises an ion channel activator (e.g., a TRPV1 channel activator, a TRPA1 channel activator, an ASIC channel activator, or combination thereof), and a plurality of pharmaceutically acceptable excipients.

In some embodiments, said test aliquot of the composition comprises a TRP channel activator or an ASIC channel activator. In some embodiments, said TRP channel activator comprises a TRPV1 activator and a TRPA1 activator. In some embodiments, said composition comprises a TRP activator and an ASIC channel activator. In certain embodiments, the TRPV1 agonist is a capsaicinoid, e.g., capsaicin. In some embodiments, the TRPV1 channel activated by said ion channel activator (e.g., a TRPV1 channel activator, TRPA1 channel activator, ASIC channel activator or combination thereof) is present on a sensory neuron in the mouth, the esophagus and/or the stomach. In some embodiments, said ion channel activator (e.g., a TRPV1 channel activator, TRPA1 channel activator, ASIC channel activator or combination thereof) increases inhibitory signaling to alpha motor neurons.

In some embodiments, the composition is formulated as a liquid or a solid. In some embodiments, the liquid is selected from the group consisting of emulsions, microemulsions, solutions, suspensions, syrups (e.g., syrup concentrates), linctuses, drops, sprays, and elixirs. In some embodiments, the composition is formulated as a solid. In some embodiments, the solid is selected from the group consisting of tablets, capsules, powders, crystals, pastes, gels, lozenges (e.g., liquid filled lozenges), gums, candies, chews, foodstuffs, dissolving strips, films, and semi-solid formulations. In some embodiments, said solid is a tablet or capsule. In some embodiments, said capsule is a hard or soft capsule.

In another aspect, the invention features a method of evaluating a composition for treating unwanted or abnormal muscle contraction, e.g., cramp, spasm, dystonia, or fasciculation, or absence of normal muscle contraction, e.g., gait abnormalities, comprising: a) acquiring, e.g. indirectly or directly, knowledge of a result of a test that shows that administration of a test aliquot of said composition alleviates a test muscle contraction in a test subject; and b) acquiring, e.g. indirectly or directly acquiring, knowledge of the effectiveness of the administration said composition to an administration subject in the treatment of unwanted or abnormal muscle contraction or absence of normal muscle contraction; wherein effectiveness in treating muscle cramp in one or both of step a and step b is indicative of usefulness of the composition for treating muscle cramp. In some embodiments, administration of the test aliquot alleviates the test muscle contraction. In some embodiments, step b is performed only if the composition alleviates the test muscle contraction in step a. In some embodiments, step a comprises performing said test. In some embodiments, step b comprises administering said composition to said administration subject. In some embodiments, step a comprises performing said test and step b comprises administering said composition to said administration subject.

In some embodiments, said test subject and administration subject are of the same species, e.g., both are rodent or both are primate, e.g., human. In some embodiments, said test subject and administration subject are the same individual. In some embodiments, said test subject and administration subject are the different individuals. In some embodiments, said test subject and administration subject are of the different species, e.g., the test species is non-human, e.g., rodent, and the administration subject is a primate, e.g., a human.

### BRIEF DESCRIPTION OF THE DRAWINGS

FIG. 1 is a series of graphs from 6 sensory neurons isolated from the trigeminal ganglia of rats that illustrate their activation by the capsicum, cinnamon, and ginger extracts that were used in the human experiments.

FIG. 2 shows the effect of the TRP-Stim beverage on cramping of the flexor hallucis brevis (FHB) of Subject A.

FIG. 3 shows the effect of the TRP-Stim beverage on cramping of the FHB of a second subject after cramping was induced.

FIG. 4 shows the effect of the TRP-Stim beverage on cramping of the FHB of a third subject tested over longer times.

FIG. 5 shows the effect of the TRP-Stim beverage on cramping of the FHB of a fourth subject.

FIG. 6 is a graph showing the effect of the TRP-Stim beverage on cramping of the gastrocnemius (calf) muscle of a fifth subject.

FIG. 7 shows the effect of the TRP-Stim beverage on cramping of the gastrocnemius (calf) muscle of a sixth subject.

FIG. 8 is a graph showing the effect of the TRP-Stim beverage on cramping of an FHB muscle in a seventh subject who experienced spontaneous cramping induced by pointing her toe.

### DETAILED DESCRIPTION OF THE INVENTION

The methods and compositions of the present invention are directed to the treatment of peripheral nervous system conditions (e.g., peripheral neuropathy), central nervous system conditions, muscle conditions and disorders (e.g., fibromyalgia, muscle spasms and cramps (e.g., nocturnal cramps), painful muscle contractions (e.g., a muscle contraction of the head or neck), neuromuscular disorders (e.g., motor neuron disease) or dystonia (e.g., cervical dystonia, blepharospasm, back spasms, or leg cramps due to spinal stenosis)), connective tissue diseases (e.g., degenerative joint disease), throat conditions (e.g., dysphagia or spasmodic dysphonias), tactile sensitivity, electrolyte imbalance and/or vitamin deficiency, respiratory conditions (e.g., asthma), cough, and sarcoidosis using a composition that an ion channel activator (e.g., a TRPV1 channel activator, a TRPA1 channel activator, an ASIC channel activator, or combination thereof).

### DEFINITIONS

The term “acidulant” as used herein refers to an acidic compound (e.g., citric acid) used to lower the pH of a composition, e.g., the pH can be lowered in the range of 2.5-6.5 (e.g., pH of 2.5, 3.0, 3.5, 4.0, 4.5, 5.0, 5.5, 6.0, or 6.5).

“Acquire” or “acquiring” as the terms are used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., applying or measuring a current to or from a subject, or capturing a signal from a subject or sample or performing a synthetic or analytical method) to obtain the value or physical entity. “Indirectly acquiring” refers to receiving the value or physical entity from another party or source (e.g., a third party laboratory that directly acquired the physical entity or value). Directly acquiring a value or physical entity includes performing a process that includes a physical change in a physical substance or the use of a machine or device. Exemplary changes include applying a current to, or measuring a current from, the muscle of a subject. Directly acquiring a value includes performing a process that uses a machine or device, e.g., a device to induce a cramp or a device to measure a parameter related to a cramp.

The term “agonist,” as used herein refers to a molecule that stimulates a biological response. In some embodiments, an agonist is an activator. For example, the activators or agonists referred to herein activate TRP ion channels, (e.g., TRPV1 ion channel).

The use of the words “a” or “an” when used in conjunction with the term “comprising” herein may mean “one,” but it is also consistent with the meaning of “one or more,” “at least one,” and “one or more than one.”

The term “administering” and “administration” refers to a mode of delivery. A daily dosage can be divided into one, two, three or more doses in a suitable form to be administered one, two, three or more times throughout a time period. In preferred embodiments of the present invention, compositions and solutions are administered orally. Where the term “composition” is used to describe a formulation that includes ion channel activators (e.g., TRPV1 channel activators, TRPA1 channel activators, ASIC channel activators, or combinations thereof), the term refers to a comestible formulation that is suitable for oral ingestion by the subject (e.g., the human subject). Exemplary compositions that include ion channel activators (e.g., TRPV1 channel activators, TRPA1 channel activators, ASIC channel activators, or combinations thereof) include solid dosage forms for oral administration (e.g., capsules, tablets, pills, dragées, crystals, pastes, gels, powders, gums, granules, chews, foodstuffs, films, and the like), liquid dosage forms for oral administration (e.g., emulsions, microemulsions, solutions, suspensions, syrups (e.g., syrup concentrates), linctuses, drops, and elixirs), ready-to-drink beverages, dry compositions that can be reconstituted with a liquid (e.g., powders, granules, or tablets that may be reconstituted with water), gels, semi-solids (e.g., ice cream, pudding, or yogurt), frozen liquids (e.g., ice pops), lozenges or hard candies, dissolving strips (e.g., an edible strip containing pullulan and compositions of the invention), and chewing gums. Other compositions are described herein.

The terms “analog” or “related analogs” as used herein refer to a substance that has a similar chemical structure to another compound, but differs from it with respect to a certain component or components.

The term “derivative” as used herein refers to a substance produced from another substance either directly or by modification or partial substitution.

“Muscle cramp” as used herein is a muscle cramp which is treated with the composition described herein. In embodiments it is not induced but rather arises spontaneously either from activity or underlying disease etiology, e.g., athletic activity or night cramp. In an embodiment, the muscle cramp comprises a cramp in a muscle other than the muscle of the test muscle cramp. The muscle cramp can be a contraction of a skeletal muscle or the smooth muscle. In an embodiment, muscle cramps occur most frequently in the muscles of the foot, calf, front of the thigh (e.g., quadricep), back of the thigh (e.g., hamstring), hands, arms (e.g., bicep or tricep), abdomen, and muscles along the rib cage. A muscle cramp that occurs in the calf muscle is also commonly known as a “charley horse.” Other common muscle cramps include exercise-induced muscle cramps, menstrual cramps, “writer's cramp,” “musician's cramp,” and night cramps (or nocturnal cramps). In an embodiment, the muscle cramp is a contraction of a muscle other than a skeletal muscle, e.g., a smooth muscle.

“Muscle spasm” as used herein refers to an involuntary contraction or a muscle, or even a few fibers of a muscle. Often the magnitude or duration of a spasm is less than that of a cramp. If the spasm is forceful and sustained, it becomes a cramp.

“Dystonia” as used herein refers to sustained muscle contractions that cause twisting and repetitive movements or abnormal postures.

“Fasciculation” as used herein refers to a small, local, involuntary muscle contraction and relaxation. Fasciculations are also commonly known as a “muscle twitch”.

The term an “effective amount” of a compound as used herein, is that amount sufficient to effect beneficial or desired results, such as the effective treatment of peripheral nervous system conditions (e.g., peripheral neuropathy), central nervous system conditions (e.g., amyotrophic lateral sclerosis), muscle conditions and disorder (e.g., fibromyalgia, muscle spasms and cramps, cervical dystonia, blepharospasm, back spasms, or leg cramps due to spinal stenosis), connective tissue disorders (e.g., degenerative joint disease), throat conditions (e.g., dysphagia or spasmodic dysphonias), and sarcoidosis, and, as such, an “effective amount” depends upon the context in which it is being applied. For example, in the context of administering an agent that activates a TRP channel (e.g., TRPV1 or TRPA1) or an ASIC channel, an effective amount of an agent is, for example, an amount sufficient to achieve an increase in TRPV1, TRPA1, and/or ASIC channel activity as compared to the response obtained without administration of the agent. The effective amount of active compound(s) used to practice the present invention can also be varied based on, for example, the age, and body weight, of the subject or the nature of the exercise.

The compositions can also include excipients that are not activators of TRPV1, TRPA1, or ASIC channels, and that are non-toxic and non-inflammatory in a subject (e.g., in a human subject). In some embodiments, the excipient(s) can provide desirable or improved physical and/or chemical properties such as stability, flow, viscosity, rate of disintegration, taste, delivery, etc. Exemplary, non-limiting excipients that can be selected from: a disintegrant (e.g., carmellose, starch, crystalline cellulose, low-substituted hydroxypropyl cellulose, and the like), a binder (e.g., gum acacia, carmellose, gelatin, crystalline cellulose, simple syrup, honey, hydroxypropyl cellulose, povidone, methylcellulose, and the like), a surfactant (e.g., polyoxyl 40 stearate, polysorbate 80, polyoxyethylene hydrogenated castor oil, and the like), an emulsifier (e.g., polyoxyl 40 stearate, sorbitan sesquioleate, polysorbate 80, sodium lauryl sulfate, lauromacrogol, gum arabic, cholesterol, stearic acid, povidone, glyceryl monostearate, and the like), a plasticizer (e.g., glycerin, propylene glycol, macrogol, and the like), a lubricant (e.g., magnesium silicate, carmellose, light anhydrous silicic acid, stearic acid, calcium stearate, magnesium stearate, talc, and the like), a sweetener (e.g., white soft sugar, honey, simple syrup, glucose, saccharin sodium, acesulfame potassium, disodium glycyrrhizinate, and the like), a pH-adjusting agent (e.g., hydrochloric acid, citric acid, sodium hydrogen carbonate, potassium hydroxide, sodium hydroxide, sodium carbonate, and the like), a preservative (e.g., benzoic acid, benzalkonium chloride, ethyl parahydroxybenzoate, butyl parahydroxybenzoate, propyl parahydroxybenzoate, methyl parahydroxybenzoate, and the like), a flavor (e.g., fennel oil, orange oil, cinnamon oil, thymol, orange peel tincture, dl-menthol, 1-menthol, eucalyptus oil, and the like), or a coloring agent (e.g., Food Red No. 2, No. 3, No. 40, No. 102, No. 104, No. 105 or No. 106, Food Yellow No. 4 or No. 5, Food Green No. 3, Food Blue No. 1 or No. 2, titanium dioxide, sodium copper chlorophyllin, turmeric, gardenia, annatto dye, kaoliang dye, and the like), or an antioxidant (e.g., ascorbic acid, sodium thiosulfate, tocopherol, sodium hydrogen sulfite, and the like), or any combination thereof.

The term “subject” as used herein refers to a mammal, including, but not limited to, a human or non-human mammal, such as a bovine, equine, canine, ovine, or feline mammal.

As used herein, and when used in reference to TRPV1, TRPA1, and/or ASIC channel activators, the term “substantially pure” refers to a composition that includes a channel activator, in which the composition is free of organic and/or inorganic species that do not activate the TRPV1, TRPA1, and/or ASIC channels, and where 60%, 65%, 70%, 75%, 80%, 85%, 90%, 95%, 97%, 98%, 99%, or 99.5% (w/w) of the composition is a particular channel activator compound. Substantially pure compositions can be prepared and analyzed using standard methods known in the art (e.g., chromatographic separation, extractions, and the like). Substantially pure compositions can include isomeric impurities (e.g., geometric isomers) and/or salts or solvates of a channel activator.

A “test muscle contraction” as used herein is a muscle contraction, typically induced, e.g., by the application of electrical current, in the subject. Stimulation can be applied to induce a muscle contraction that recapitulates a naturally-occurring muscle cramp, muscle spasm, dystonia, or fasciculation, e.g., a test muscle cramp, a test muscle spasm, a test muscle dystonia, or a test muscle fasciculation. In embodiments, the test muscle cramp comprises a cramp in the flexor hallucis brevis muscle. In some embodiments, efficacy in inducing a test muscle cramp in a subject is indicative of efficacy in treating muscle cramp, e.g., with a composition described herein. In other embodiments, efficacy in treating the test muscle cramp is indicative of efficacy in treating muscle cramp, spasticity, dystonias, or fasciculations. The terms “treat,” “treating,” or “ameliorating” as used herein refer to administering a composition for therapeutic purposes or administering treatment to a subject already suffering from a disorder to improve the subject's condition.

The terms “treating a condition or disorder” or “ameliorating a condition or disorder” as used herein refer to the condition or disorder (e.g., peripheral nervous system conditions (e.g., peripheral neuropathy), central nervous system conditions, muscle conditions and disorders (e.g., fibromyalgia, muscle spasms and cramps (e.g., nocturnal cramps), painful muscle contractions (e.g., a muscle contraction of the head or neck), neuromuscular disorders (e.g., motor neuron disease) or dystonia (e.g., cervical dystonia, blepharospasm, back spasms, or leg cramps due to spinal stenosis)), connective tissue diseases (e.g., degenerative joint disease), throat conditions (e.g., dysphagia or spasmodic dysphonias), tactile sensitivity, electrolyte imbalance and/or vitamin deficiency, respiratory conditions (e.g., asthma), cough, and sarcoidosis) and the symptoms associated with the condition or disorder are, e.g., alleviated, reduced, cured, or placed in a state of remission. As compared with an equivalent untreated control, such amelioration or degree of treatment is at least 5%, 10%, 20%, 30%, 40%, 50%, 60%, 70%, 80%, 90%, 95%, 99%, or 100%, as measured by any standard technique.

The term “viscosity” as used herein refers to a measurement of a fluid's internal resistance to flow (e.g., “thickness”). Viscosity is generally expressed in centipoise (cP) or pascal-seconds.

Other features and advantages of the invention will be apparent from the detailed description and from the claims.

### Compositions

The compositions described herein are comestible formulations suitable for administration to a subject (e.g., a human) and include one or more ion channel activators (e.g., activators of TRPV1, TRPA1, or ASIC channels) as well as one or more optional excipients as described herein. Exemplary, non-limiting compositions include those that are solid dosage forms for oral administration (e.g., tablets, capsules, powders, crystals, pastes, gels, lozenges (e.g., liquid filled lozenges), gums, candies, chews, foodstuffs, films, and the like), liquid dosage forms for oral administration (e.g., emulsions, microemulsions, solutions, suspensions, syrups (e.g., syrup concentrates), linctuses, drops, sprays, elixirs, and the like), ready-to-drink beverages, dry compositions that can be reconstituted with a liquid (e.g., powders, granules, or tablets that may be reconstituted with water), gels, semi-solids (e.g., ice cream, pudding, or yogurt), frozen liquids (e.g., ice pops), lozenges or hard candies, dissolving strips (e.g., an edible strip containing pullulan and compositions of the invention), and chewing gums.

### TRP Channels and ASIC Channels

Transient Receptor Protein (TRP) channels are a family of ion channels that are generally expressed on the cell surface. Members of the TRP channel family share some structural similarity and are organized in sub-families, comprising TRPA, TRPC, TRPV, TRPM, TRPML, TRMPN, and TRPP. Each of these sub-families comprise subunit genes, which include, for example, TRPV1, TRPV2, TRPV4, TRPV3, TRPV5, TRPV6, TRPA1, TRPP3, TRPP2, TRPP5, TRPC4, TRPC5, TRPC1, TRPC3, TRPC7, TRPC6, TRPM1, TRPM3, TRPM6, TRPM7, TRPM4, TRPM5, TRPM2, TRPM8, TRPML1, TRPML3, and TRPML2. The compositions described herein may comprise at least one activator or agonist of any of the TRP channels.

Acid-sensing ion channels (ASIC) are neuronal voltage-insensitive cationic channels that are activated by extracellular protons. ASIC channels are primarily expressed in the nervous system, and conduct mostly Na⁺. There are four ASIC channel genes, ASIC1, ASIC2, ASIC3 and ASIC4, which encode at least six ASIC channels, ASIC3, ASCI4 and splice variants of ASIC1, and ASIC2, ASIC1a, ASIC1b, ASIC2a, ASIC2b. The compositions described herein may comprise at least one agonist of any of the ASIC channels.

### TRPV1 Channel Small Molecule Activators

Compounds that activate TRPV1 that may be used in the compositions of the present invention include, naturally occurring and non-naturally occurring compounds (e.g., synthetic analogs and derivatives of naturally occurring compounds), including but not limited to those described below.

### Naturally Occurring Small Molecule Activators of TRPV1

TRPV1 channel activators include naturally occurring compounds. Examples include: curcumin, piperines, piperyline, piperettine, piperolein A, piperolein B, piperanine, warburganal, N-arachidonoyl-dopamine (NADA), N-acylphenolamine, polygodial, isovelleral, guaiacol, eugenol, zingerone, triprenyl phenols (e.g., scutigeral), gingerols, shogaols, N-oleoylethanolamine, oleoylethanolamide, N-oleoyldopamine, 3-methyl-N-oleoyldopamine, oleamide, N-arachidonolylserine, N-acyltaurines (e.g., N-arachylnaurine, N-acylsalsolinols (e.g., N-arachidonoylsalsolinol), miogadial, miogatrial, polygodial, sanshools, evodiamine, anandamide, and 4-hydroxynonenal.

### Non-Naturally Occurring Small Molecule Activators of TRPV1

TRPV1 channel activators include non-naturally occurring compounds that are derived by synthetic methods (e.g., by combining two or more naturally occurring small molecule activator as described above or by creating an artificial compound that does not exist in nature). Examples of non-naturally occurring small molecule activators of TRPV1 include but are not limited to: ricinoleic acid derivatives, including 12,4′-diphenylacetyl rinvanil, 12-phenylacetyl rinvanil, 2′,2′,2′-trichloroethyl ricinoleate, 2′,2′,2′-trichloroethyl 12-phenylacetyl ricinoleate, 12-phenylacetyl ricinoleic acid, 12-phenylacetyl rinvanil, 2′,2′,2′-trichloroethyl 12-benzoyl ricinoleate, 12-benzoyl ricinoleic acid, 12-benzoyl rinvanil, 9,10-methylen-12,4′-diphenyl acetyl rinvanil, 9,10-methylen-12-phenylacetyl rinvanil, 4′-(2-aminoethyl)-12-phenylacetyl rinvanil (hydrochloride), and 10-epoxy-12-phenylacetyl rinvanil; N-vanillylmyristamide; N-(3-methoxy-4-hydroxybenzyl) oleamide; N-[(4-(2-aminoethoxy)-3-methoxyphenyl)methyl]-9Z-octadecenamide; N-(9Z-octadecenyl)-3-methoxy-4-hydroxyphenylacetamide; octyl 3,4-dihydroxyphenylacetamide, octyl 4-hydroxyphenylacetamide; N-N′-(3-methoxy-4-aminoethoxy-benzyl)-(4-tert-butyl-benzyl)-urea; [1-[2-(1-adamantyl)ethyl]-1-pentyl-3-[3-(4-pyridyl)propyl]urea], N-vanillyl-alkanedienamides, N-vanillyl-alkanedienyls; N-vanillyl-cis-monounsaturated alkenamides (e.g., N-vanillyl-9Z-octadecenamide (N-vanillyloleamide) and N-[(4-acetoxy-3-methoxyphenyl)methyl]-9Z-octadecenamide); N-[(4-(2-aminoethoxy)-3-methoxyphenyl)methyl]-9Z-octa-decanamide; N-oleyl-homovanillamide; acesulfame-K; cyclamate; flufenamic acid dopamide and other dopamine amides of fenamic acids; and urea derivatives (e.g., 1-[2-(1-adamantyl)ethyl]-1-pentyl-3-[3-(4-pyridyl)propyl]urea, 1-[2-(1-adamantyl)ethyl]-3-[3-(4-pyridyl)propyl]-1-(3,3,3-trifluoropropyl)urea, 1-[3-(1-adamantyl)propyl]-1-propyl]-3-[3-(4-pyridyl)propyl]urea, 1-[2-(1-adamantyl)ethyl]-3-[1-methyl-3-(4-pyridyl)propyl]-1-pentylurea, 1-[2-(1-adamantyl)ethyl]-3-[2-methyl-3-(4-pyridyl)propyl]-1-pentylurea, (+)-1-[2-(1-adamantyl)ethyl]-3-[2-methyl-3-(4-pyridyl)propyl]-1-pentylurea, and (E)-1-[2-(1-adamantyl)ethyl]-1-pentyl-3-[3-(4-pyridyl)-2-propenyl]urea).

Additional TRPV1 channel activators are described, for example, in U.S. Pat. Nos. 8,642,775; 8,546,352; 8,338,457; 8,263,093; 8,252,816; 7,632,519; 7,446,226; 7,429,673; 7,407,950; 6,872,748; 6,022,718; 5,962,532; 5,762,963; 5,403,868; 5,290,816; 5,221,692; 5,021,450; 4,812,446; 4,599,342; 4,564,633; 4,544,669; 4,544,668; 4,532,139; 4,493,848; 4,424,205; 4,313,958; in U.S. Patent Application Publication Nos. 2013/0090359; 2007/0293703; 2007/0167524; 2006/0240097; and 2005/0085652; and in WO 00/50387; Appendino et al. Curr. Pharm. Des. 2008, 14: 2-17; Huang et al., Proc Natl Acad Sci USA 2002, 299: 8400-8405; Högestatt et al., J Biol Chem 2005, 280 (36): 31405-12; and Vriens et al. Mol Pharmacol 2009, 75-1262-1279; each of which is incorporated by reference.

### Capsaicinoids, Capsinoids, and Analogs Thereof as TRPV1 Channel Activators

Capsaicinoids, and analogs thereof, capsinoids, and analogs thereof are compounds that can activate TRPV1 channels and may be used in the compositions of the present invention. These compounds can be naturally occurring and non-naturally occurring compounds (e.g., synthetic analogs and derivatives of naturally occurring compounds), including but not limited to those described below.

Suitable capsaicinoids, capsinoids, and related analogs and derivatives and combinations thereof for use in the compositions and methods of the present invention can be naturally occurring and include: capsaicin, dihydrocapsaicin, nordihydrocapsaicin, homodihydrocapsaicin, homocapsaicin, nonivamide, pseudocapsaicin, resiniferatoxin, tinyatoxin, capsiate, dihydrocapsiate, nordihydrocapsiate, norcapsaicin, capsiconiate, dihydrocapsiconiate and other coniferyl esters, capsiconinoid, and 3-hydroxyacetanilide.

Capsaicinoids and related analogs and derivatives also include non-naturally occurring compounds that are derived by synthetic methods (e.g., by combining two or more naturally occurring capsaicinoids as described above or by creating an artificial compound that does not exist in nature). Examples of non-naturally occurring capsaicinoids include but are not limited to: esters of capsaicinoids (e.g., aliphatic esters, hydrophilic esters, and the like including 4-[((6E)-8-methylnon-6-enoylamino)methyl]-2-methyoxyphenyl formate, 4-[((6E)-8-methylnon-6-enoylamino)methyl]-2-methyoxyphenyl acetate, 4-[((6E)-8-methylnon-6-enoylamino)methyl]-2-methyoxyphenyl propanoate, 4-[((6E)-8-methylnon-6-enoylamino)methyl]-2-methyoxyphenyl butanoate, 4-[((6E)-8-methylnon-6-enoylamino)methyl]-2-methoxyphenyl 2,2-dimethylpropanoate, 4-[((6E)-8-methylnon-6-enoylamino)methyl]-2-methyoxyphenyl octadecanoate, and 4-[((6E)-8-methylnon-6-enoylamino)methyl]-2-methyoxyphenyl {4-[((6E)-8-methylnon-6-enoylamino)methyl]-2-methoxyphenoxy}formate, ester derivatives of capsinoids, (e.g. homovanillyl 8-methylnonanoate), 3-(3-methoxy-4-hydroxyphenyl)propyl 8-methylnonanoate, 8-methylnonyl homovanillate, substituted benzyl ester derivatives of capsinoids (e.g. 8-methylnonanoic acid-substituted benzyl ester derivative), isobutylamides (e.g. heptanoylisobutylamide), guaiacylamides (e.g. heptanoyl guaiacylamide), halogenated capsaicin analogs, phenylcapsaicins (e.g. 7-phenylhept-6-yne-acid-4-hydroxy-3-methoxybenzylamide), N-vanillyl fatty acid amides (e.g. dohevanil), denatonium capsaicinate, capsaicin derivatives (e.g. N-[4-(2-aminoethoxy)-3-methoxybenzyl]-N′-[2-(4-chlorophenyl)ethyl]thiourea, N-[4-(2-aminoethoxy)-3-methoxybenzyl]-N′-[2-(4-fluorophenyl)ethyl]thiourea, N-[4-(2-aminoethoxy)-3-methoxybenzyl]-N′-[2-(2,4-dichlorophenyl)ethyl]thiourea, N-[4-(2-aminoethoxy)-3-methoxybenzyl]-N′-[2-(4-benzyloxyphenyl)ethyl]thiourea, N-[4-(2-aminoethoxy)-3-methoxybenzyl]-N′-[2-(4-(n-octyloxy)phenyl)ethyl]thiourea, and N-[4-(2-aminoethoxy)-3-methoxybenzyl]-N′-[4-n-octyloxybenzyl]thiourea), N-phenylmethylalkynamide capsaicin derivatives; ether linked and relatively nonpungent analogues of N-nonanoyl vanillylamide (e.g. N-(4-O-glycerol-3-methoxybenzyl)-nonamide, N-nonanoyl vanillylamide-4-glyceryl ether, N-(4-O-acetic acid sodium)-3-methoxybenzyl-nonamide, sodium N-nonanoyl vanillylamide-4-O-acetate, and N-(4-O-glycol-3-methoxybenzyl)-nonamide), N-nonanoyl vanillylamide-4-glycol ether), compounds prepared by combining phorbol related diterpenes and homovanillac acid analogs via esterification at the exocyclic hydroxy group of the diterpene (e.g. 20-homovanillyl-mezerein and 20-homovanillyl-12-deoxyphorbol-13-phenylacetate), civamide (N-[(4-hydroxy-3-methoxyphenyl)-methyl]-8-methyl-(Z)-6-nonemamide), nuvanil, capsavanil, olvanil, arvanil, and palvanil (N-palmitoyl-vanillamide).

Additional capsaicinoids are described, for example, in U.S. Pat. Nos. 8,652,497; 8,642,657; 8,420,600; 8,309,060; 8,212,068; 7,981,460; 7,943,666; 7,446,226; 7,034,058; 6,333,421; 5,891,919; 5,403,868; 5,290,816; 5,221,692; 5,021,450; 4,812,446; 4,493,848; 4,564,633; and 4,313,958.

Additional capsaicinoids and capsinoids are exemplified in U.S. Provisional Application Nos. 61/979,405 and 61/797,423, which are hereby incorporated by reference. TRPV1 channel activators for use in the compositions and methods described herein can also be identified using standard methodology, as described, for example, in U.S. Patent Application Publication No. 2003/0104085, which is hereby incorporated by reference. Exemplary assays for identification of TRPV1 channel activators include, without limitation, receptor binding assays; functional assessments of stimulation of calcium influx or membrane potential in cells expressing the TRPV1 receptor; assays for the ability to induce cell death in such cells (e.g., selective ablation of C-fiber neurons); and other assays known in the art.

In addition, the TRPV1 channel activator may be an acidulant (e.g., acetic acid, phosphoric acid, citric acid, malic acid, succinic acid, tartaric acid, lactic acid, fumaric acid, or ascorbic acid) maintaining a low pH in the range of 2.5-6.5 (e.g., pH of 2.5, 3.0, 3.5, 4.0, 4.5, 5.0, 5.5, 6.0, or 6.5).

A TRPV1 channel activator may be present in a composition of the invention at a concentration range of about 0.001% to 10% by weight by weight (w/w) based on the total weight of the composition (e.g., 0.001, 0.005, 0.01, 0.1, 0.5, 1, 2, 3, 4, 5, 6, 7, 8, 9, or 10%) or at a concentration range of about 0.001% to 10% by weight by volume (w/v) based on the total volume of the composition (e.g., 0.001, 0.005, 0.01, 0.1, 0.5, 1, 2, 3, 4, 5, 6, 7, 8, 9, or 10%), though a TRPV1 channel activator may be present in lower or higher concentrations (e.g., less than 0.01%, e.g., 0.008%, 0.005%, 0.004%, 0.001% (w/w) or (w/v), or more than 10%, e.g., 12%, 15%, 20%, 30%, 35%, 40%, 50% (w/w) or (w/v)). The TRPV1 channel activator may be present at a concentration range of about 20 mg to 500 mg per unit dosage (e.g., 23 mg, 25 mg, 30 mg, 35 mg, 40 mg, 45 mg, 50 mg, 60 mg, 70 mg, 100 mg, 150 mg, 200 mg, 250 mg, 300 mg, 350 mg, 400 mg, 420 mg, or 450 mg).

### TRPA1 Channel Small Molecule Activators

Compounds that activate TRPA1 that may be used in the compositions of the present invention include, naturally occurring and non-naturally occurring compounds (e.g., synthetic analogs and derivatives of naturally occurring compounds), including but not limited to those described below.

### Naturally Occurring Small Molecule Activators of TRPA1

TRPA1 channel activators include mustard oil, isothiocyanate compounds (e.g., allyl isothiocyanate), acrolein, farnesyl thiosalicylic acid, Δ₉-tetrahydrocannabinol (THC), eugenol, ginger, gingerol, gingerols, shogaols, cinnamaldehyde, cinnamon oil, wintergreen oil, clove oil, allicin, diallyl sulfide, diallyl disulfide, diallyl trisulfide, sanshools, farnesyl thiosalicylic acid, farnesyl thioacetic acid, thymol, limonene, bradykinin, alkenyl aldehyde 4-HNE, cyclopentenone prostaglandin (15dPGJ2, 15-deoxy-Δ12,14-prostaglandin J2), acetaldehyde, 4-hydroxy-2-nonenal, isovelleral, and o-cresol.

### Non-Naturally Occurring Small Molecule Activators of TRPA1

In another embodiment, the TRPA1 channel activators include non-naturally occurring compounds that are derived by synthetic methods (e.g., by combining two or more naturally occurring small molecule activator as described above or by creating an artificial compound that does not exist in nature). Non-naturally occurring small molecule activators of TRPA1 include by are not limited to: dibenzoazepine and dibenzooxazepine derivatives, dibenz[b,fJ-1,4]oxazepine, formalin, 6,11-dihydro-5H-dibenzo[b,e]azepine-10-carboxylic acid methyl ester, propofol, icilin, formalin, 6,11-dihydro-5H-dibenzo[b,e]azepine-10-carboxylic acid methyl ester, and 4-isobutylamino-2-[4-(tetrahydro-pyran-3-ylmethyl)-piperazin-1-yl]-pyrimidine-5-carboxylic acid benzylamide.

Other activators of TRPA1 are described, for example, in Harteneck et al., Adv Exp Med Biol. 2011, 704:87-106; Viana et al. Expert Opin. Ther. Pat. 2009, 19(12):1787-99; Bandell et al., Neuron, 2004, 41 (6): 840-857; McNamara et al., Proc. Natl. Acad. Sci. USA 2007, 104(33): 13525-13530; Trevisiani et al., Proc. Natl. Acad. Sci. USA 2007, 104:13519-13524; Cruz-Orengo et al., Molecular Pain 2008, 4:30; Ryckmans et al., Bioorg Med Chem Lett 2011, 21: 4857-4859; Macpherson et al. Nature 2007, 445: 541-545; Jordt et al., Nature 2004, 427(6971):260-265; Escalera et al., J Biol Chem, 2008, 283: 24136-24144; and U.S. Pat. Nos. 8,623,880; 8,614,201; 8,461,145; 7,960,130; and 7,674,594, each of which is incorporated by reference.

Methods for identifying TRPA1 channel activators are known in the art and are described, for example, in U.S. Pat. Nos. 7,674,594; 7,662,576; 7,465,581; and U.S. Patent Publication Nos. 2014/0024725 and 2007/0196866.

A TRPA1 channel activator may be present in a composition of the invention at a concentration range of about 0.001% to 10% by weight by weight (w/w) based on the total weight of the composition (e.g., 0.001, 0.005, 0.01, 0.1, 0.5, 1, 2, 3, 4, 5, 6, 7, 8, 9, or 10%) or at a concentration range of about 0.001% to 10% by weight by volume (w/v) based on the total volume of the composition (e.g., 0.001, 0.005, 0.01, 0.1, 0.5, 1, 2, 3, 4, 5, 6, 7, 8, 9, or 10%), though a TRPA1 channel activator may be present in lower or higher concentrations (e.g., less than 0.001%, e.g., 0.0008%, 0.0005%, 0.0004%, 0.0001% (w/w) or (w/v), or more than 10%, e.g., 12%, 15%, 20%, 30%, 35%, 40%, 50% (w/w) or (w/v)). The TRPA1 channel activator may be present at a concentration range of about 20 mg to 500 mg per unit dosage (e.g., 23 mg, 25 mg, 30 mg, 35 mg, 40 mg, 45 mg, 50 mg, 60 mg, 70 mg, 100 mg, 150 mg, 200 mg, 250 mg, 300 mg, 350 mg, 400 mg, 420 mg, 450 mg).

### Other TRP Channels Activators

Other TRP channel activators or agonists suitable for the methods and compositions described herein are known in the art. In one embodiment, the TRP channel agonist may be non-selective and may activate more than one TRP channel. For example, carvacrol, a compound present in oregano, activates both TRPA1 and TRPV3. In another example, icilin and menthol activate TRPA1 and TRPM8. Naturally occurring and synthetic derivatives and analogs of carvacrol, icilin, or menthol are suitable for use in the compositions and methods of the present invention. Suitable agonists or activators of TRP channels for use in the compositions of the present invention or administered in accordance with the methods of the present invention are disclosed herein. The various agonists for the TRP channel family members listed below is not to be construed as an all-inclusive list, but is merely presented to provide examples of additional TRP agonists.

Examples of TRPV4 agonists include, but are not limited to, 4-alpha-phorbol-12,13 didecanoate (4α-PDD), GSK1016790A, 5′,6′-epoxyeicosatrienoic (5′6′-EET), 8′,9′-epoxyeicosatrienoic (8′9′-EET), APP44-1, R1747, arachidonic acid (AA), 12-O-tetradecanoylphorbol-13-acetate (TPA), phorbol 12-myristate 13-acetate (PMA), bisandrographalide (BAA), anandamide, and any of the compounds disclosed in WO 2006/029209 (e.g., a compound of Formula I, II, IIa, or III, N-{(1S)-1-[({(4R)-1-[(4-chlorophenyl)sulfonyl]-3-oxohexahydro-1-azepin-4-yl}amino)carbonyl]-3-methylbutyl}-1-benzothiophen-2-carboxamide, N-{(1S)-1-[({(4R)-1-[(4-fluorophenyl)sulfonyl]-3-oxohexahydro-1H-azepin-4-yl}amino)carbonyl]-3-methylbutyl}-1-benzothiophen-2-carboxamide, N-{(1S)-1-[({(4R)-1-[(2-cyanophenyl)sulfonyl]-3-oxohexahydro-H-azepin-4-yl}amino)carbonyl]-3-methylbutyl}-1-methyl-1H-indole-2-carboxamide, N-{(S)-1-[({(4R)-1-[(2-cyanophenyl)sulfonyl]hexahydro-1H-azepin-4-yl}amino)carbonyl]-3-methylbutyl}-1-methyl-1H-indole-2-carboxamide), or N-(4-hydroxyphenyl)-5Z,8Z,11Z,14Z-eicosatetraenamide (AM404).

Examples of TRPC6 agonists or activators include, but are not limited to, 1-oleoyl-2-acetyl-sn-glycerol (OAC), carbachol, diacylglycerol (DAG), 1,2-didecanoylglycerol, flufenamate/flufenamic acid, niflumate/niflumic acid, hyperforin, and the compounds disclosed in WO 2010/015965 (e.g., a compound of Formula IV, compound IX, compound X, compound XI, compounds XII).

Examples of TRPM6 agonists or activators include, but are not limited to 2-aminoethoxydiphenyl borate (2-APB).

Examples of TRPV2 agonists or activators include, but are not limited to, diphenylborinic anhydride (DPBA), delta-9-tetrahydrocannabinol (A⁹-THC or THC), cannabiniol (CBN), cannabidiol (CBP), 2-APB, probenecid, 0-1821, 11-hydroxy-A⁹-tetrahydrocannabinol, nabilone, CP55940, HU-210, HU-21 1/dexanabinol, HU-331, HU-308, JWH-015, WIN55, 212-2, 2-arachidonoylglycerol (2-AG), Arvil, PEA, AM404, 0-1918, and JWH-133.

Examples of TRPV3 agonists or activators include, but are not limited to incensole, incensole acetate, a compound disclosed in WO 2008/065666 (e.g., a compound of Formula I or Formula II, compound IA), menthol, eugenol, dihydrocarveol, carveol, thymol, vanillin, ethyl vanillin, cinnemaldehyde, 2 aminoethoxydiphenyl borate (2-APB), diphenylamine (DPA), diphenylborinic anhydride (DPBA), camphor, (+)-borneol, (−)-isopinocampheol, (−)-fenchone, (−)-trans-pinocarveol, isoborneol, (+)-camphorquinone, (−)-a-thujone, α-pinene oxide, 1,8-cineole/eucalyptol, 6-tert-butyl-m-cresol, carvacrol, p-xylenol, cresol, propofol, p-cymene, (−)-isopulegol, (−)-carvone, (+)-dihydrocarvone, (−)-menthone, (+)-linalool, geraniol, farnesyl pyrophosphate, farnesyl diphosphate, isopentenyl pyrophosphate, and 1-isopropyl-4-methyl-bicyclo[3.1.0]hexan-4-ol.

The TRP channel agonist or activator may also be an analog or derivative of any of the TRP channel activators described herein.

A TRP channel agonist or activator may be present in a composition of the invention at a concentration range of about 0.001% to 10% by weight by weight based on the total volume of the composition (e.g., 0.001, 0.005, 0.01, 0.1, 0.5, 1, 2, 3, 4, 5, 6, 7, 8, 9, or 10%), though a TRP channel agonist or activator may be present in lower or higher concentrations.

A TRP channel agonist or activator can also be identified using standard methodology. Exemplary assays known in the art for identification of agonists of any TRP channel in the TRP family include, without limitation, receptor binding assays; functional assessments of stimulation of calcium influx or membrane potential in cells expressing the TRPV1 receptor; assays for the ability to induce cell death in such cells (e.g., selective ablation of C-fiber neurons); and other assays known in the art.

### ASIC Channel Activators

ASIC channels are activated by low pH. The pH of a composition of the present invention that includes an ASIC channel activator may be in the range of 2.5-6.5 (e.g., pH of 2.5, 3.0, 3.5, 4.0, 4.5, 5.0, 5.5, 6.0, or 6.5). The pH may be adjusted within this range by any means acceptable for compositions that are intended to be ingested by a subject. Exemplary acidulants are acetic acid, phosphoric acid, citric acid, malic acid, succinic acid, lactic acid, tartaric acid, fumaric acid, and ascorbic acid. The acidulant may be present in a composition of the invention at a concentration range of about 0.001% to 10% by weight based on the total volume of the composition (e.g., about 0.001, 0.005, 0.01, 0.1, 0.5, 1, 2, 3, 4, 5, 6, 7, 8, 9, or 10%), though the acidulant may be present in lower or higher concentrations.

### Additional Components of the Composition

The composition of the present invention may additionally include, for example, electrolytes (e.g., potassium salt or other salts), sweeteners, flavoring and coloring agents, vitamins, minerals, preservatives, viscosity modifiers, and antioxidants as described below. Other exemplary excipients are described in Handbook of Pharmaceutical Excipients, 6^{th} Edition, Rowe et al., Eds., Pharmaceutical Press (2009).

### Viscosity and Viscosity Modifiers

Viscosity is the ratio of shear stress to shear rate, expressed as dynes-second/cm², or poise. A centipoise (cP) is one one-hundredth of a poise.

The composition of the present invention may have a viscosity greater than water (i.e., about 1.0 cP at 20° C.), e.g., about 100, 200, 300, 400, 500, 1000, 1500, 2000, 2500, 3000, 3500, 4000, 4500, 5000, 6000, 7000, 8000, 9000 cP or more. If a consistency of corn syrup is desired, viscosities in the range of about 2500 cP are suitable. If a consistency of a soft gel or honey is desired, viscosities in the range of about 10000 cP to about 15000 cP are suitable. For pudding-like products, viscosities in the range of about 30000 cP to about 38000 cP are desirable. Viscosity of the compositions of the present invention may be measured with, e.g., a rheometer or viscometer, though additional methods of measuring viscosity are known in the art.

Viscosity modifiers may be added to compositions of the present invention. Such viscosity modifiers include, for example, collagen, gellan gum, carbohydrate gel-forming polymers, carob bean gum, locust bean gum, carrageenan, alginates (e.g., alginic acid, sodium alginate, potassium alginate, ammonium alginate, and calcium alginate), agar, guar gum, xanthan gum, carboxymethyl cellulose, clear starch, pectin, gelatin, arrowroot, cornstarch, katakuri starch, potato starch, sago, tapioca, furcellaran, karo syrup (e.g., light karo syrup and dark karo syrup), and sodium pyrophosphate. A viscosity modifier may be present in the composition in an amount of from about 0.01% to about 10% by weight based on the total volume of the composition (e.g., about 0.01, about 0.1, about 0.5, about 1, about 2%, about 3%, about 4%, about 5%, about 6%, about 7%, about 8%, about 9%, or about 10%), though the viscosity modifier may be present in lower or higher concentrations (e.g., about 10%, about 20%, about 30%, about 40%, about 50%, about 60%, about 70%, about 80%, or about 90%). In some embodiments, the viscosity modifier is present in the composition from about 40% to about 60% (e.g, about 50%).

### Electrolytes

Exemplary electrolytes include potassium salts, chloride salts, bromide salts, sodium salts, magnesium salts, calcium salts, citrate salts, acetate salts, phosphate salts, salicylates, bicarbonate salts, lactate salts, sulphate salts, tartrate salts, benzoate salts, selenite salts, molybdate salts, iodide salts, oxides, and combinations thereof. An electrolyte may be present in a composition of the invention at a concentration range of about 0.01% to about 10% by weight based on the total volume of the composition (e.g., about 0.01%, about 0.02%, about 0.03%, about 0.04%, about 0.05%, about 0.1%, about 0.5%, about 1%, about 2%, about 3%, about 4%, about 5%, about 6%, about 7%, about 8%, about 9%, or about 10%), though an electrolyte may be present in lower or higher concentrations.

In certain embodiments, the compositions of the present invention include high concentrations of potassium (e.g., potassium chloride). The concentration of potassium in the composition may be, e.g., about 0.01%, about 0.02%, about 0.03%, about 0.04%, about 0.05%, about 0.1%, about 0.5%, about 1%, about 2%, about 3%, about 4%, about 5%, about 6%, or about 7% or more by weight based on the total volume of the composition.

In certain embodiments, the compositions of the present invention include high concentrations of magnesium (e.g., magnesium chloride). The concentration of magnesium in the composition may be, e.g., about 0.01%, about 0.02%, about 0.03%, about 0.04%, about 0.05%, about 0.1%, about 0.5%, about 1%, about 2%, about 3%, about 4%, about 5%, about 6%, or about 7% or more by weight based on the total volume of the composition.

### Sweeteners

Sweeteners may be included in the compositions of the invention. Exemplary sweeteners include corn syrup (e.g., high fructose corn syrup or karo syrup), mannose, maltose, glucose polymers, sucrose (e.g., cane sugar or beet sugar), glucose, dextrose, lactose, galactose, fructose, polysaccharides (e.g., malodextrins), rice syrup, honey, and natural fruit juices (e.g., orange juice, papaya juice, pineapple juice, apple juice, grape juice, apricot juice, pear juice, tomato juice, agave nectar, or cranberry juice). Additionally, non- or low-caloric sweeteners can be used in the compositions of the invention. Examples of such non-caloric or low-caloric sweeteners include, but are not limited to, saccharin, cyclamates, acetosulfam, sorbitol, sucralose, xylitol, erythritol, Stevia extract, L-aspartyl-L-phenyl-alanine ester (e.g., aspartame), L-aspartyl-D-alanine alkyl amides, L-aspartyl-L-1-hydroxymethylalkaneamide, and L-aspartyl-1-hydroxyethylalkaneamide. Sweeteners may be present in a composition of the invention at a concentration range of about 2% to about 20% by weight based on the total volume of the composition (e.g., about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%), though sweeteners may be present in lower or higher concentrations.

### Flavoring and Coloring Agents

Exemplary flavoring agents include almond oil, amaretto oil, anethole, anise oil, benzaldehyde, blackberry, black walnut oil, blueberry, caraway, caraway oil, cardamom oil, cardamom seed, cherry juice, cherry syrup, cinnamon, cinnamon oil, cinnamon water, citric acid, citric acid syrup, clove oil, cocoa, coriander oil, dextrose, eriodictyon, ethyl acetate, ethyl vanillin, fennel oil, ginger, glucose, glycerin, glycyrrhiza, grape, honey, lavender oil, lemon oil, lime, mannitol, methyl salicylate, myristica oil, orange oil, orange peel, orange syrup, peppermint, peppermint oil, peppermint water, phenylethyl alcohol, pineapple, raspberry juice, raspberry syrup, rosemary oil, rose oil, rose water, sarsaparilla syrup, sorbitol, spearmint, spearmint oil, strawberry, sucrose, thyme oil, tolu balsam, vanilla, vanillin, and wild cherry syrup. Additional flavoring agents may be found in Food Chemicals Codex and Fenaroli's Handbook of Flavor Ingredients.

Small amounts of a coloring agent may be utilized in the compositions of the present invention. Coloring agents include, e.g., beta-carotene, riboflavin dyes, FD&C dyes (e.g., Yellow No. 5, Blue No. 1, Blue No. 2, and Red No. 40), FD&C lakes, chlorophylls and chlorophyllins, caramel coloring, annatto, cochineal, turmeric, saffron, paprika, and fruit, vegetable, and/or plant extracts (e.g., grape, black currant, aronia, carrot, beetroot, red cabbage, elderberry, and hibiscus extracts). The amount of coloring agent used will vary depending on the agents used in the composition and the color intensity desired in the finished product. The amount of coloring agent to be used can be readily determined by one skilled in the art.

### Vitamins and Minerals

Non-limiting examples of vitamins and minerals that may be included in the compositions of the present invention include, e.g., choline bitartate, niacinamide, thiamin, folic acid, d-calcium pantothenate, biotin, vitamin A, vitamin C, vitamin B₁ hydrochloride, vitamin B₂, vitamin B₃, vitamin B₆ hydrochloride, vitamin B₁₂, vitamin D, vitamin E acetate, vitamin K, and salts of calcium, potassium, magnesium, zinc, iodine, iron, and copper. When included in a composition of the invention, the composition contains at least about 5%, about 10%, about 15%, about 20%, about 25%, about 30%, about 35%, about 40%, about 45%, or about 50% of the U.S. recommended daily intake (RDI) for such vitamins and minerals.

### Preservatives

A preservative may additionally be utilized in the compositions described herein. Exemplary preservatives include, for example, sorbate, benzoate, and polyphosphate preservatives (e.g., sorbic acid, benzoic acid, calcium sorbate, sodium sorbate, potassium sorbate, calcium benzoate, sodium benzoate, potassium benzoate, and mixtures thereof). When included in a composition of the invention, the preservative is included at levels from about 0.0005% to about 0.5% (e.g., about 0.0005%, about 0.001%, about 0.005%, about 0.01%, about 0.05%, about 0.1%, or about 0.5%) by weight based on the total volume of the composition, though preservatives may be present in lower or higher concentrations.

### Antioxidants

An antioxidant agent may also be included in the compositions to, for example, reduce exercise-induced oxidative stress. Exemplary antioxidants include vitamin C and vitamin E; beta-carotene, lutein, or other carotenoids; cyanidin, delphinidin, malvidin, or other anthocyanidins; apigenin, luteolin, or other flavones; hesperitin, naringenin, or other flavonones; isorhamnetin, quercetin, kaempferol or other flavonols; and epigallocatechin-3-gallate, epicatechin, thearubigins, or other flavan-3-ols. When included in a composition of the invention, the antioxidant is included at levels from about 0.0005% to about 0.5% (e.g., about 0.0005%, about 0.001%, about 0.005%, about 0.01%, about 0.05%, about 0.1%, or about 0.5%) by weight based on the total volume of the composition, though antioxidants may be present in lower or higher concentrations.

Additional components of the compositions described herein may include amino acids (e.g., leucine, isoleucine, lysine, methionine, phenylalanine, threonine, tryptophan, and valine), stimulants (e.g., caffeine), emulsifying agents, carbon dioxide (e.g., to carbonate a liquid composition), stabilizers, humectants, anticaking agents, or herbal extracts. These components may be included at levels from about 0.0005% to about 25% (e.g., about 0.0005%, about 0.001%, about 0.005%, about 0.01%, about 0.05%, about 0.1%, about 0.5%, about 1%, about 5%, about 10%, about 15%, about 20%, or about 25%) by weight based on the total volume of the composition, though an additional component may be present in lower or higher concentrations.

### Formulations and Methods of Preparing Compositions

The compositions and solutions of the present invention may be formulated as ready-to-drink beverages, concentrates (e.g., syrups), dry compositions (e.g., powders, granules, or tablets that may be reconstituted with a liquid (e.g., with water), gels, solids, semi-solids (e.g., ice cream, pudding, or yogurt), frozen liquids (e.g., ice pops), lozenges or hard candies, dissolving strips (e.g., an edible strip containing pullulan and compositions of the invention), and chewing gum. Formulation of these compositions may require the use of a formulation base, which is a substance or material mixed with or added to the ion channel activator and pharmaceutically acceptable excipient in order to achieve the desired form.

In solid dosage forms for oral administration (e.g., tablets, capsules, powders, crystals, pastes, gels, lozenges (e.g., liquid filled lozenges), gums, candies, chews, foodstuffs, dissolving strips, films, semi-solid formulations, dragées, and the like), the compositions of the invention are mixed with a pharmaceutically-acceptable carrier, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

In some embodiments, the compositions may be in the form of a dry powder, granule, tablet, or capsule that may be reconstituted in a specified amount of a liquid. The dried components may be mixed together and milled (e.g., to create a homogenous powder) or mixed in aqueous solution and dried by using methods known to one of skill in the art. Dried powders or granules may be “loose” or fashioned into tablets.

In other embodiments, the compositions of the present invention may be in the form of a gel or paste further comprising a humectant (e.g., glycerin, propylene glycol, lithium chloride, alpha hydroxy acids, diols, urea, quillaia, polyols, sugar alcohols (e.g., sorbitol, glycerol, xylitol, mannitol), glyceryl triacetate, or neoagarobiose), a gum (e.g., xanthan gum, guar gum), an abrasive (e.g., silica, (e.g., Zeodent®)), a plasticizer, an additive (e.g., a sweetener, preservative, buffering agent, penetration agent, surfactant, coloring agent, flavoring agent, cleaning agent, and the like) or a thickener (e.g., silica (e.g., Zeodent®)). These additional components may be present in the composition of the invention from about 0.5% to about 99% (e.g., about 0.5%, about 0.1%, about 0.5%, about 1%, about 5%, about 10%, about 20%, about 30% about 40%, about 50%, about 75%, about 90%, about 95%, or about 99%) by weight based on the total volume of the composition, though these components may be present in lower or higher concentrations.

The gel or paste may be further packaged on or within a delivery device such as a bioadhesive strip, patch, film, or may be provided for application directly to the oral cavity (e.g., mucosal surfaces (e.g., in the mouth, nose, or throat), teeth, gums, or lips). For example, a paste or gel can be packaged in a unit that contains between about 0.1 ounces to about 16 ounces of the paste or gel. For example, the packaging can contain about 0.1 ounces, about 0.25 ounces, about 0.5 ounces, about 1 ounce, about 2 ounces, about 3 ounces, about 4 ounces, about 5 ounces, about 6 ounces, about 7 ounces, about 8 ounces, about 9 ounces, about 10 ounces, about 11 ounces, about 12 ounces, about 13 ounces, about 14 ounces, about 15 ounces, or about 16 ounces.

To make pills containing the composition of the invention, the powdered ingredients are mixed together with a binding agent, such as acacia or tragacanth, and are then made into a plastic mass by incorporation of any liquid drugs and addition of an inert liquid. The resulting mass, known as a pill mass, is then rolled into spheres and coated with talc, gelatin, or sugar.

To make tablets, the ion channel activators (e.g., TRPV1 channel activators, TRPA1 channel activators, ASIC channel activators, or combination thereof) are mixed with suitable diluents, such as dextrin, lactose, salt, starch, or synthetic substances, designed to ensure disintegration of the tablet in the body. To prevent sticking in the machine, a lubricant such as liquid paraffin, stearic acid, talc, or a synthetic substance is usually added. Furthermore, it is essential that the tablet machines are fed with the drug mixture in a free-flowing form to ensure complete filling of the molds. To achieve this, the composition mixture is customarily granulated by mechanically forcing pellets of the mixture through a sheet of perforated-metal. The granulated mixture is fed into the tablet machine, which feeds the correct dose into a cavity, the mixture then being compressed by means of a punch that fits into the cavity. To be successful, the tablet maker must choose correct diluents and lubricants, prepare suitable granules, and obtain the right degree of compression in the tablet machine. Excessive compression may mean that the tablet will not disintegrate in the body; insufficient compression results in fragile tablets that may break, causing inaccurate dosage. Coatings of various types may be applied to the tablet to protect the ingredients from deterioration, to hide the taste of certain components, to control the release of the active components from the tablet, or to produce a more attractive tablet. For sugar coatings, a concentrated sucrose syrup containing suspended starch, calcium or magnesium carbonate, or other suitable substance is applied, each successive layer being dried before the application of the next. After the final layer is dried, it is highly polished to give an elegant finish. Sugar coatings provide both protection and a sweet taste. Film coatings can also be used, in which a very thin transparent film, usually a cellulose derivative, is applied. Enteric coating is designed to resist solution in the stomach and to dissolve in the more alkaline intestinal fluid. Many substances have been used for enteric coatings, one of which is cellulose acetate phthalate (cellacephate). In the manufacture of layered tablets, incorporating two or more drugs, a compressed tablet is fed to a second machine where another layer is compressed around it. In this way, drugs normally incompatible may be formulated in the same tablet.

Other solid dosages such as lozenges, candies, dragées, or pastilles disintegrate or dissolve in the mouth, slowly releasing the active ingredient (e.g., any of the TRPV1, TRPA1, or ASIC channel activators described herein). The base usually consists of a mixture of sugar and gum or gelatin. Lozenges are generally manufactured by compression techniques, while pastilles are fabricated by fusion and the use of molds. Dry extracts are prepared by the methods for fluid extracts, followed by evaporation, usually under reduced pressure, either to a pilular consistency or to dryness. Dry extracts are usually granulated by being passed through a sieve and may be used for the preparation of tablets.

Liquid dosage forms for oral administration include pharmaceutically acceptable emulsions, microemulsions, solutions, suspensions, syrups (e.g., syrup concentrates), linctuses, drops, and elixirs. In addition to the active ingredient (e.g., any of the TRPV1 channel activators, TRPA1 channel activators, ASIC channel activators, or combinations thereof, described herein), the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The compositions and solutions described herein may be bottled or packaged in, for example, glass bottles, plastic bottles and containers (e.g., polyethylene terephthalate or foil-lined ethylene vinyl alcohol), metal cans (e.g., coated aluminum or steel), lined cardboard containers, pouches, packs, wrappers, or any other packaging known to one of skill in the art. For example, a ready-to-drink beverage can be bottled or packaged in a unit that contains between about 10-1000 mL of the beverage. For example, the packaging can contain about 10 mL, 20 mL, 50 mL, 100 mL, 200 mL, 300 mL, 400 mL, 500 mL, 600 mL, 700 mL, 800 mL, 900 mL, or 1000 mL of the beverage. Alternatively, the packaging can contain 200 mL, 250 mL, 330 mL, 350 mL, 355 mL, 375 mL, 440 mL, or 500 mL of the beverage. A ready-to-drink beverage can also be bottled or packaged in a unit that contains between about 1-32 fluid ounces of beverage (e.g., the unit may contain about 1, 2, 5, 6.75, 8, 8.3, 8.4, 8.45, 9.6, 10, 12, 15, 15.5, 16, 18.6, 20, 23, 24, or 32 fluid ounces). Where a shelf-stable composition or solution is desired, the packaging is appropriately sterilized before being filled by the pasteurized, ultra-pasteurized, or sterilized composition or solution. Where required for mutual stability of two or more components (for example if a component is unstable at low pH), the packaging may feature multiple containers that can be mixed shortly before ingestion or that can be consumed serially.

Formulations for oral use may also be provided as chewable tablets, or as hard gelatin capsules wherein the active ingredient (e.g., any of the ion channel activators (e.g., TRPV1 channel activators, TRPA1 channel activators, ASIC channel activators, or combinations thereof), described herein) is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

### Oil-Based Formulations

The compositions of the invention can be formulated as an oil-based formulation for oral administration.

In one embodiment, the oil-based formulation includes a formulation base composition including an oil and a lipophilic additive, which can be solid or pasty at room temperature. The lipophilic additive can include waxes, fatty acid mono-, di- or triglycerides, fatty acids and polyethylene glycols and the polyethylene glycol fatty acid esters, as well as their mixtures and can be present in ranges from about 5 to 20% by weight or the composition (e.g., about 5%, about 6%, about 10%, about 15%, about 17%, about 18%, about 19%, or about 20%). The waxes can be beeswax, candelilla wax, carnauba wax, polyethylene oxide wax or petroleum wax (or microcrystalline wax). The fatty acid mono-, di-, or triglycerides can have different degrees of esterification. The fatty acids can be selected from among palmitic acid, stearic acid, or behenic acid and their calcium, sodium, potassium or magnesium salts. The polyethylene glycols and fatty acid polyethylene glycol esters can have a molecular weight of between about 600 to 6000. The oil can include vegetable oils such as soya oil, sunflower oil, corn oil, olive oil or nut oil, and among the mineral oils such as liquid paraffin, as well as their mixtures. The oil-based formulations can be present in the form of a soft or hard capsule and can be prepared by traditional techniques known in the art. In one such technique, the lipophilic additive is incorporated into the oil which is heated at a temperature sufficiently high to melt the lipophilic additive completely and obtain a homogeneous mixture. After cooling to approximately 50° C., the other components such as the ion channel activator (e.g., TRPV1 channel activator, TRPA1 channel activator, ASIC channel activator, or combination thereof), described herein are incorporated into this mixture with stirring. The mixture thus obtained is cooled to a temperature between 25 and 40° C., and optionally, soft or hard capsules are filled with this mixture. For a detailed discussion of lipids and lipid-based formulations see, for example, Porter et al., Nat Rev Drug Discov 2007, 6(3):231-248.

In another embodiment, the ion channel activators (e.g., TRPV1 channel activators, TRPA1 channel activators, ASIC channel activators, or combinations thereof) can be formulated as an oil for topical administration. Generally, the activators described herein at concentration ranges of about >20% to 95% (w/w) are solubilized in a solvent capable of solubilizing the ion channel activator. Solvents that may be used include volatile solvents (e.g., methanol, ethanol, acetone, isopropanol, n-propanol, cyclohexane and alkanes with molecular weight less than dodecane (C12)), semi-volatile solvents (e.g., volatile essential oils such as clove oil, tea tree oil, sesame oil, and cineole), and non-volatile solvents (e.g., polyethylene glycol 400, Lutrol® (polyethylene polyoxypropylene block copolymer available from BASF), glyceryl monooleate, glycerin, lanolin, low melting waxes, sesquiterpenes and alkanes, alkenes, alkanoic and alkenoic acids >C28). The oils may further include a crystal lization inhibitor, for example, polyvinylpyrrolidone, Luvitol® BD 10 P (BASF), povidone and its derivatives; dextrin derivatives, polyethylene glycol, polypropylene glycol, mannitol and glycerin, and mono and diglycerides of essential oils, polyglycerin fatty acid esters, sucrose palmitic acid ester, pentaerythritol ester of wood rosin (Pentalyn A®), and Eudagrits®. Crystallization inhibitors may range from about 0.1 to 10% w/w. The oils of the ion channel activators described herein may be administered orally as an oil.

### Controlled Release Formulations

It is also within the scope of the invention to provide compositions that are formulated for modified release (e.g., delayed release, prolonged and/or slow release, extended release, or rapid release) of the ion channel activators (e.g., TRPV1 channel activators, TRPA1 channel activators, ASIC channel activators, or combinations thereof) to reduce gastrointestinal side-effects. Such compositions are well-known in the art and include e.g., diffusion-controlled drug delivery systems, osmotic pressure controlled drug delivery systems, or erodible drug delivery systems. Exemplary delivery systems are the SQZgel™ (MacroMed, Inc.), comprising a pH-sensitive polymer mixture combined with an outer coating in which the acidic environment of the stomach causes the polymer to imbibe with water and swell, entrapping the ion channel activator. Upon entering the higher pH of the intestines, the polymer slowly shrinks, or “squeezes” at a “dialed-in” rate releasing the active composition in a sustained manner); the Egalet® extrusion based technology (Egalet A/S), comprising a biodegradable coat and a matrix, including the ion channel activator, which is surface erodible, hydrophobic, and composed of PEG-stearate); Diffucaps/Surecaps (small beads approximately 1 mm or less in diameter that can be incorporated into hard gelatin capsules, where the ion channel activator release profiles are created by layering drug onto a neutral core such as sugar spheres, crystals, or granules followed by a rate-controlling, functional membrane); and MeltDose®, which involves formulating solubilized, individual molecules into tablets).

The ion channel activators (e.g., TRPV1 channel activators, TRPA1 channel activators, ASIC channel activators, or combinations thereof) described herein can be formulated for pH controlled release. Examples of suitable formulation principles are, for example, compositions provided with an enteric coating or hydrogels of a type described in U.S. Pat. Nos. 6,537,584 and 5,484,610, which are hereby incorporated by reference.

Another suitable formulation includes the formulation of the ion channel activators (e.g., TRPV1 channel activators, TRPA1 channel activators, ASIC channel activators, or combinations thereof) together with vitamin E concentrate in soft or hard gelatin capsules. Another specific example of a suitable formulation includes formulation of the ion channel activators (e.g., TRPV1 channel activators, TRPA1 channel activators, ASIC channel activators, or combinations thereof) together with ethanol, tocopherolethylene glycol 1000 succinate (TPGS), corn oil, and wax in soft or hard gelatin capsules. Variations of this formulation can include ethanol, TPGS, corn oil, and polyglycolized glycerides (e.g., Gelucire) in soft or hard gelatin capsules. The resulting product can be a semi-solid or solid dosage form. The release rate of this formulation is dependent on degradation due to lipases in the intestine.

A further example of a suitable formulation is an oral pulsed dose drug delivery system. This dosage form can be perceived as a modified form of the Schering Repetab tablets. A portion of the composition of the present invention is put in the core of a tablet. The core can for example, be made by conventional wet granulation or continuous granulation such as extrusion followed by compaction of the granulate into tablets. The core is then coated using an appropriate technology, for example, by air-suspension using an enteric coating polymer such as Eudragits. The first releasing dose is compression coated on the core or air-suspension coated either with the enteric coat or on top of the enteric coat. In an embodiment of the invention, the first releasing dose is air-suspension coated with the enteric coat. In a further embodiment of the invention, the first releasing dose is compression coated on the core, in order to avoid release of the composition according to the invention prior to the degradation of the enteric coat, such degradation typically occurring at pH values higher than those found in the gastric ventricle (i.e., the degradation of the enteric coat typically occurs after passage of the gastric ventricle). Another example of a suitable formulation is an oral sustained drug delivery system. In this delivery system, the core can for example, be made by conventional wet granulation or continuous granulation such as extrusion followed by compaction of the granulate into tablets. The core is then coated using appropriate technology, for example, by air-suspension using ethylcellulose and a hydrophilic excipient such as hydroxyl propyl cellulose (HPC).

In some embodiments, the compositions of the inventions can include the ion channel activators (e.g., TRPV1 channel activators, TRPA1 channel activators, ASIC channel activators, or combinations thereof) in the form of micro-crystals with hydrophilic surfaces. The micro-crystals can be film coated directly in order to achieve a sustained release formulation. The compositions of the invention can also be complexed with genuine cyclodextrins and cyclodextrin-derivatives (e.g., alkyl- and hydroxyalkyl-derivatives or sulfobutyl-derivatives). The complexation is achieved by methods known in the art. Complexation can lead to a higher solubility and a higher dissolution rate and higher bioavailability.

In other embodiments, the composition can include a pharmaceutically acceptable excipient that is an agent for delayed or controlled release of the ion channel activators (e.g., TRPV1 channel activators, TRPA1 channel activators, ASIC channel activators, or combinations thereof). In some aspects, the agent is a water-soluble polymer, including but not limited to, hydroxypropylmethyl cellulose, hydroxypropyl cellulose, hydroxypropyl methyl cellulose (HPMC), methyl cellulose, and carboxymethyl cellulose.

The ion channel activators (e.g., TRPV1 channel activators, TRPA1 channel activators, ASIC channel activators, or combinations thereof) described herein can be targeted to the mucus/mucosal lining of the mouth, tongue, nose, or gastrointestinal tract (GIT) through the use of bioadhesives. A bioadhesive is defined as a synthetic or biological material which is capable of adhering to a biological substrate or tissue. When the biological substrate is mucus, the term “mucoadhesive” has been employed. When the biological tissue involved is the mouth or the stomach, the terms “buccoadhesive” or “gastroadhesive” have been employed. Bioadhesives can remain attached to the biological substrate for an extended period of time. The period of time a bioadhesive is required to remain attached to a biological substrate will vary according to the target site and the condition being treated. Other delivery systems that can target the TRP or ASIC channel activators described herein to the colon include, but are not limited to:
(a) covalent linkage of the ion channel activator with the carrier to form a prodrug that is stable in the stomach and small intestine and releases the ion channel activator in the large intestine upon enzymatic transformation by the intestinal microflora; examples of these prodrugs include azo-conjugates, cyclodextrin-conjugates, glycoside-conjugates, glucuronate conjugates, dextran-conjugates, polypeptide and polymeric conjugates;
(b) approaches to deliver intact molecule to the colon, such as coating with pH-sensitive polymers to release the ion channel activator at neutral to alkaline pH, or coating with biodegradable polymers which release the ion channel activator upon degradation by the bacteria in the colon;
(c) embedding the ion channel activator in biodegradable matrices and hydrogels which release the ion channel activator in response to the pH or biodegradation;
(d) time released systems where once the multicoated formulation passes the stomach, the ion channel activator is released after a lag time of 3-5 hrs which is equivalent to the transit time of the small intestine;
(e) using redox-sensitive polymers where a combination of azo and disulfide polymers provide ion channel activator release in response to the redox potential of the colon;
(f) osmotic controlled delivery where the ion channel activator is released through semi-permeable membrane due to osmotic pressure.

### Micro and Nanoparticle Formulations

In one embodiment, the ion channel activator (e.g., TRPV1 channel activator, TRPA1 channel activator, ASIC channel activator, or combination thereof)-loaded nano- and micro-particles for sustained release are formulated by the nano-precipitation or the oil-in-water single emulsion solvent evaporation/extraction method. First, the ion channel activator (e.g., TRPV1 channel activator, TRPA1 channel activator, ASIC channel activator, or combinations thereof)-loaded poly(lactic-co-glycolic acid) (PLGA) nanoparticles are prepared by the nano-precipitation method. The volume of oil-water ratio can be adjusted (e.g., from about 1:2 to 1:5, e.g., about 1:2, 1:3, 1:4, or 1:5), and size of the nanoparticles can be selected (e.g., from about 162+/−3 nm to 153+/−3 nm, e.g., about 154, 155, 156, 157, 158, 159, 160, 161, or 162) to increase drug loading efficiency and drug release period. To get a more sustained release, a modified single emulsion method can be applied with biocompatible polymers such as polylactic acid (PLLA), polyhydroxy butyrate (PHB), polyglycolic acid (PGA), PLGA, and poly-ε-caprolactone (PCL).

In another embodiment, stomach specific mucoadhesive nanoparticles (SSMN) can be used to improve controlled delivery of the ion channel activators (e.g., TRPV1 channel activators, TRPA1 channel activators, ASIC channel activators, or combinations thereof) by continuous release of the activator for a prolonged period to its absorption site to ensure optimal bioavailability. The ion channel activators (e.g., TRPV1 channel activators, TRPA1 channel activators, ASIC channel activators, or combinations thereof) with a narrow absorption window are mostly associated with improved absorption at the jejunum and ileum due to enhanced absorption properties of these sites (e.g., large surface area), or because of enhanced solubility in the stomach as opposed to the more distal parts of the gastrointestinal tract. Ion channel activators that may benefit from using stomach specific mucoadhesive nanoparticles includes those that act locally in the stomach, those with low solubility at high pH values, those that are primarily absorbed in the stomach, those with a narrow window of absorption, e.g., ion channel activators that are absorbed mainly from the proximal part of the small intestine, those that absorb rapidly from the gastro intestinal tract, those that degrade in the colon, and those that are unstable in intestinal fluids. Longer residence time in the stomach could be advantageous for local action especially in the upper part of the small intestine. A list of the micro and nano carriers for mucosal delivery applications is described in Table 1.

TABLE 1
Micro and nano carriers for mucosal delivery

| | | Zeta potential | Loading |
|---|---|---|---|
| Carrier | Size (μm) | (mV) | method |
| Chitosan | 0.215 | 20.7 | Encapsulation |
| Chitosan | | | Encapsulation |
| Chitosan/HPMCPa | 0.255 | 30.1 | Encapsulation |
| Chitosan/dextran sulfate | 0.479-1.612 | 21.5 to 3.2 | Encapsulation |
| Chitosan/dextran sulfate | 0.527, 1.577 | 20.6, 11.5 | Encapsulation |
| Chitosan/eudragit | 0.196 | 29.51 | Encapsulation |
| L100-55 | | | |
| Chitosan/lecithin | 0.121-0.347 | 7.5-32.7 | Encapsulation |
| Chitosan/alginate | 0.779.1.858 | | Encapsulation |
| Chitosan/alginate | 0.748 | 5.6 | Encapsulation |
| Lauryl succinyl chitosan | 0.315-1.090 | | Encapsulation |
| TMCO-60% b | | | Encapsulation |
| N-trimethyl | 0.102-0.168 | 12.3-18.8 | Self assembly |
| chitosan-cysteine | | | |
| Poly(lactic acid)-chitosan | 0.065 | 5 | Encapsulation |
| MePEG-PLA-CSc | 0.094 | 13 | Encapsulation |
| DEAPA-PVA-g-PLLAd | 0.200-0.400 | 15-35 | Self assembly |
| PLGAe | 0.216--1.145 | | Encapsulation |
| PLGAe | 0.15 | | Encapsulation |
| WGA modified PLGAf | 0.232-0.240 | −4.2 to −2.6 | Encapsulation |
| Polyacrylic | 0.191-0.228 | −29.3 to −7.3 | Encapsulation |
| acid/spermine | | | |
| Polyacrylic acid/MgCl2 | 0.278-23.4 | | Encapsulation |
| Lipid nanoparticles | 0.2 | −50.3 | Encapsulation |
| Lipid nanoparticles/PEG | 0.207-0.226 | −36.6 to −34.8 | Encapsulation |
| Lipid | 0.538 | 29.2 | Encapsulation |
| nanoparticles/chitosan | | | |
| Solid lipid nanoparticles | 0.050-0.064 | −46.3 to −38 | Encapsulation |
| WGA-N-glut- | 0.058-0.075 | 57.7-75.3 | Encapsulation |
| PEmodified SLNsp | | | |

### Routes of Administration

The compositions described herein may be administered to a subject in a variety of forms depending on the selected route of administration, as will be understood by those skilled in the art and as relating to the particular disease or condition to be treated. The compositions used in the methods described herein may be administered, for example, by topical, enteral, or parenteral applications. Topical applications include but are not limited to epicutaneous, inhalation, enema, eye drops, ear drops, and applications through mucous membranes in the body. Enteral applications include oral administration, rectal administration, vaginal administration, and gastric feeding tubes. Parenteral administration includes intravenous, intraarterial, intracapsular, intraorbital, intracardiac, intradermal, transtracheal, subcuticular, intraarticular, subcapsular, subarachnoid, intraspinal, epidural, intrastemal, intraperitoneal, subcutaneous, intramuscular, transepithelial, nasal, intrapulmonary, intrathecal, rectal, and topical modes of administration. Parenteral administration may be by continuous infusion over a selected period of time.

In some embodiments of the current invention, the ion channel activator (e.g., TRPV1 channel activators, TRPA1 channel activators, ASIC channel activators, or combinations thereof) is administered through the oral cavity to achieve mucosal and transmucosal effects. Exemplary applications include buccal, nasal, intradermal, inhalational, topical, subcutaneous, sublingual, sublabial, and insufflation administrations. Compositions of the current invention may include a penetration enhancer to increase the bioavailability of the ion channel activator within the oral cavity. Exemplary penetration enhances include surfactants (e.g., anionic surfactants (e.g, sodium lauryl sulfate), cationic surfactants (e.g., cetyl pyridinium chloride), and nonionic surfactants (e.g., poloxamer, Brij, Span, Myrj, Tween)), bile salts (e.g., sodium glycocholate, sodium taurodeoxycholate, sodium taurocholate), fatty acids (e.g., oleic acid, caprylic acid, lauric acid, lysophosphatidylcholine, phosphatidylcholine), cyclodextrins (e.g, α-, β-, or γ-cyclodextrans, methylated cyclodextrins), chelators (e.g., EDTA, citric acid, sodium salicylate, methyl salicylates), polymers (e.g., positively charged polymers (e.g., chitosan, trimethyl chitosan)), and cationic compounds (e.g., poly L-arginine, L-lysine).

For intravenous or intrathecal delivery or direct injection, the composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

The choice of the route of administration will depend on whether a local or systemic effect is to be achieved. For example, for local effects, the composition can be formulated for topical administration and applied directly where its action is desired. For systemic, long term effects, the composition can be formulated for enteral administration and given via the digestive tract. For systemic, immediate and/or short term effects, the composition can be formulated for parenteral administration and given by routes other than through the digestive tract.

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection moldings are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

### Dosage

The compositions of the present invention are formulated into acceptable dosage forms by conventional methods known to those of skill in the art. Actual dosage levels of the active ingredients in the compositions of the present invention may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present invention employed, the route of administration, the time of administration, the rate of absorption of the particular agent being employed, the duration of the treatment, other drugs, substances, and/or materials used in combination with the particular compositions employed, the age, sex, weight, condition, general health and prior medical history of the subject being treated, and like factors well known in the medical arts. A physician or veterinarian having ordinary skill in the art can readily determine and prescribe the effective amount of the composition required. For example, the physician or veterinarian can start doses of the substances of the invention employed in the composition at levels lower than that required in order to achieve the desired therapeutic effect and gradually increase the dosage until the desired effect is achieved. In general, a suitable daily dose of a composition of the invention will be that amount of the substance which is the lowest dose effective to produce a therapeutic effect. Such an effective dose will generally depend upon the factors described above. Preferably, the effective daily dose of a therapeutic composition may be administered as two, three, four, five, six or more sub-doses administered separately at appropriate intervals throughout the day, optionally, in unit dosage forms.

Preferred therapeutic dosage levels are between about 500 mg to about 1000 mg (e.g., about 500 mg, 520 mg, 500 mg, 550 mg, 600 mg, 650 mg, 700 mg, 750 mg, 800 mg, 850 mg, 900 mg, 950 mg, or 1000 mg) of the composition per day administered orally to adults of average weight afflicted with most of the symptoms, syndromes and conditions described herein. Preferred prophylactic dosage levels are between about 100 mg to about 1000 mg (e.g., about 110 mg, 140 mg, 200 mg, 250 mg, 300 mg, 350 mg, 400 mg, 460 mg, 700 mg, 750 mg, 800 mg, 850 mg, 900 mg, 950 mg, or 1000 mg). The dose may also be titrated (e.g., the dose may be escalated gradually until signs of gastrointestinal toxicity appear, such as diarrhea or nausea).

The frequency of treatment may also vary. The subject can be treated one or more times per day (e.g., once, twice, three, four or more times) or every so-many hours (e.g., about every 2, 4, 6, 8, 12, or 24 hours). The composition can be administered 1 or 2 times per 24 hours. The time course of treatment may be of varying duration, e.g., for two, three, four, five, six, seven, eight, nine, ten, or more days, two weeks, 1 month, 2 months, 4 months, 6 months, 8 months, 10 months, or more than one year. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

### Foodstuff and Food Supplements

The present invention also relates to the use of the composition as foodstuff, food supplement or dietetic product (a foodstuff intended for a particular diet). In particular, the composition can be incorporated into foodstuffs which are industrially produced or craftsmen-prepared, such as oils, butter, margarine, bread spreads, or baked goods. It can also be presented in the form of a powder for dilution in water or food bars.

The composition of the invention can further be administered in combination with a dietary supplement to promote and/or maintain general health. Examples of dietary supplements include, but are not limited to, a vitamin (e.g., Vitamin A, Vitamin B₁, B₂, B₃, B₅, B₆, B₇, B₉, B₁₂, Vitamin C, Vitamin D, Vitamin E, and Vitamin K), a mineral (e.g., potassium, chlorine, sodium, calcium, magnesium, phosphorus, zinc, iron, manganese, copper, iodine, selenium, and molybdenum), an herb or botanical (e.g., St. John's-wort, kava, Shilajit, and Chinese herbal medicines), an amino acid (e.g., glycine, serine, methionine, cysteine, aspartic acid, glutamic acid, glutamine, tryptophan, and phenylalanine), and a concentrate, constituent, extract, and/or a combination of any of the above.

### Conditions and Disorders

The compositions of the invention may be useful for treating any of the conditions and disorders described herein.

### Peripheral Nervous System (PNS) Conditions

Compositions of the invention can be used to treat conditions affecting the peripheral nervous system (PNS). These conditions include: diseases, disorders, or injuries to the peripheral nervous system include but are not limited to: cramp fasciculation syndrome, Isaacs' Syndrome or neuromyotonia (NMT), peripheral neuropathy (e.g., diabetic neuropathy), carpal tunnel syndrome, or EBV infection. Other peripheral nervous system diseases and conditions include but are not limited to: amyloid neuropathies, diabetic neuropathies, nerve compression syndromes, peripheral nervous system neoplasms, brachial plexux neuropathies, Guillain-Barre syndrome, neuralgia, polyneuropathies, complex regional pain syndromes, mononeuropathies, neuritis, acrodynia, neurofibromatosis, hand-arm vibration syndrome, pain insensitivity, and Tarlov cysts.

### Central Nervous System (CNS) Conditions

The compositions of the invention can be used to treat conditions affecting the central nervous system (CNS). These conditions include: diseases, disorders, and injuries to the central nervous system due to tumor, multiple sclerosis, spasticity due to cerebral palsy, stroke, motor neuron disease, spinal injury or stenosis. There are many other central nervous system diseases and conditions, including infections of the central nervous system such as encephalitis and poliomyelitis, early-onset neurological disorders including ADHD and autism, late-onset neurodegenerative diseases such as Alzheimer's disease, Parkinson's disease, and essential tremor, autoimmune and inflammatory diseases such as multiple sclerosis and acute disseminated encephalomyelitis, genetic disorders such as Krabbe's disease and Huntington's disease, as well as amyotrophic lateral sclerosis and adrenoleukodystrophy. Other diseases of the CNS include but are not limited to: catalepsy, epilepsy, meningitis, migraine, tropical spastic paraparesis, arachnoid cysts, locked-in syndrome, and Tourette's syndrome. Anxiety disorders may also be characterized as a CNS condition. Anxiety disorders can be classified into: generalized anxiety disorder, phobic disorders, panic disorders, agoraphobia, social anxiety disorder, obsessive-compulsive disorder, post-traumatic stress disorder, separation anxiety, and situational anxiety.

### Tactile Sensitivity

The compositions of the invention may be useful in treating tactile sensitivity or tactile defensiveness (TD). TD refers to patterns of observable behavioral and emotional responses which are aversive, negative, and out of proportion to certain types of tactile stimuli that are often found by most people to be non-painful. TD is a sensory integrative dysfunction in which the brain is unable to process and use information through the senses. Tactile sensitivity can result from conditions such as autism, dyspraxia, neuralgia, panic or anxiety disorders, or from venomous bites or stings.

Electrolyte Imbalance and/or Vitamin Deficiency

The compositions of the invention may be useful in treating conditions associated with an electrolyte imbalance and/or vitamin deficiency. Examples of such condition include, but are not limited to: hyponatremia, hypernatremia, hyperkalemia, hypokalemia, hypercalcemia, hypocalcemia, hyperchloremia, hypochloremia, hypermagnesemia, hypomagnesemia, hyperphosphatemia, hypophosphatemia, kidney disease, rickets, scurvy, beriberi, pellagra, calcium deficiency, eating disorders, vitamin D deficiency, vitamin A deficiency, biotin deficiency, ariboflavinosis, vitamin K deficiencies, hypocobalaminemia, paraesthesia, night blindness, magnesium or thiamine deficiency, hypoparathyroidism, medullary cystic disease, and adrenocortical carcinoma.

### Muscle Conditions and Disorders

The compositions and methods described herein include treating a subject having been diagnosed with or identified as having a muscle condition or disorder. Exemplary disorders include night cramps, multiple sclerosis, dystonia, spinal cord spasticity, neuromuscular disorders, including muscle pain and cramping associated with a neuromuscular disorder, central nervous system disorders or injuries (e.g., spinal cord injury, brain injury, or stroke), muscle cramps, muscle spasms, and fasciculations.

In some embodiments, the compositions of the invention are also useful for treating painful muscle contraction of the head or neck as in tension, cluster or migraine headache, back spasms, leg cramps due to spinal stenosis, skeletal muscle pain, muscle pain (e.g., fibromyalgia) and spasms (e.g., nocturnal cramps), spasms and cramps due to treatment with dialysis, diuretics, β-blockers, statins, fibrates, β2-agonists, ACE inhibitors, ARBs and anti-psychotic medications, muscle claudication pain due to inactivity or restriction as seen in “economy class syndrome”, paralysis, peripheral artery disease or immobilization, and neuromuscular diseases.

Neuromuscular disease can be caused by circulatory problems, stroke, immunological and autoimmune disorders, the failure of the electrical insulation surrounding nerves myelin, genetic/hereditary disorders, such as Huntington's disease, certain rare tumors, the failure of the connections between the nerves and the muscle fibers, and exposure to pernicious environmental chemicals, poisoning (e.g., heavy metal poisoning). Some neuromuscular diseases are caused either by viral infections or by attack by little-known pernicious proteins called prions. Diseases of the motor end plate include myasthenia gravis, a form of muscle weakness due to antibodies to the acetylcholine receptor, and its related condition Lambert-Eaton myasthenic syndrome (LEMS). Tetanus and botulism are bacterial infections in which bacterial toxins cause increased or decreased muscle tone, respectively. Myopathies are all diseases primarily resulting in muscular degeneration, rather than affecting the nerves themselves (e.g., nemaline myopathy, centronuclear myopathy, mitochondrial myopathies, inflammatory myopathies, familial periodic paralysis, or drug-induced myopathyies). Muscular dystrophies, including Duchenne's and Becker's, are a large group of diseases, many of them hereditary or resulting from genetic mutations, where the muscle integrity is disrupted. They lead to progressive loss of strength and decreased life span. Also within the scope of the invention is treatment of Guillain-Barre syndrome and inflammatory muscle disorders, such as polymyalgia rheumatic, polymyositis, dermatomyositis, inclusion body myositis, and rhabdomyolysis. Additional neuromuscular disorders include, but are not limited to, multiple sclerosis, spinal cord spasticity, spinal muscle atrophy, myasthenia gravis, spinal cord injury, traumatic brain injury, cerebral palsy, hereditary spastic paraplegia, motor neuron disease (e.g., amyotrophic lateral sclerosis, primary lateral sclerosis, progressive muscular atrophy, progressive bulbar palsy, pseudobulbar palsy, spinal muscular atrophy, progressive spinobulbar muscular atrophy (e.g., Kennedy's disease), or post-polio syndrome), neuralgia, fibromyalgia, Machado-Joseph disease, cramp fasciculation syndrome, carpal tunnel syndrome, acrodynia, neurofibromatosis, neuromyotonias (e.g., focal neuromyotonia, Isaacs' syndrome), peripheral neuropathy, piriformis syndrome, plexopathy (e.g., Brachial plexopathy or Lumbosacral plexobathy), radiculopathy (e.g., lower lumbar radiculopathy), and encephalitis.

Dystonia is a neurological condition that affects a muscle or group of muscles in a specific part of the body causing involuntary muscular contractions and abnormal postures. Types of dystonia include: focal dystonia, multifocal dystonia, segmental dystonia, generalized dystonia (e.g., torsion dystonia or idiopathic torsion dystonia), hemidystonia, blepharospasm, psychogenic dystonia, cervical dystonia, acute dystonic reaction, and vegetative-vascular dystonia. The methods and compositions described herein may be useful for cervical dystonia, cranial dystonia, laryngeal dystonia, and hand dystonia. Causes for the disorder include, but are not limited to, inheritance, birth-related or physical trauma, infection, poisoning (e.g., lead poisoning), or reaction to some pharmaceutical agents, e.g., neuroleptics. Treatment is difficult and has been limited to minimizing symptoms of the disorder, such as muscle cramping. The methods and compositions described herein may be useful for focal dystonia, blepharospasm, cervical dystonia, cranial dystonia, laryngeal dystonia, and hand dystonia.

In one embodiment, the subject has been diagnosed or identified as having multiple sclerosis. Multiple sclerosis (MS) is also known as disseminated sclerosis and encephalomyelitis disseminate. MS is an inflammatory disease in which the insulating sheaths of nerve cells in the brain and spinal cord are damaged, thereby disrupting the ability of the nervous system to communicate. The three main characteristics of MS are the formation of lesions in the central nervous system (also called plaques), inflammation, and the destruction of myelin sheaths of neurons. Symptoms of MS can include muscle spasms and muscle weakness.

In some embodiments, the subject has been diagnosed with or identified as having night cramps (also known as nocturnal cramps). Night cramps are spontaneous muscle contractions that occur during sleep, can be very painful, and often recur throughout the night. Elderly people, e.g., over 50 years of age, are at higher risk for experiencing night cramps.

In one embodiment, the subject has been diagnosed with or identified as having spinal cord spasticity. Spasticity is the uncontrolled tightening or contracting of the muscles that is common in individuals with spinal cord injuries and a variety of nervous system diseases. Spasticity is usually defined as a velocity-dependent increase in the tonic stretch reflex (muscle tone) with exaggerated tendon jerks, clonus, and spasms, resulting from the hyperexcitability of the stretch reflex. About 65%-78% of the spinal cord injury population have some amount of spasticity, and it is more common in cervical (neck) than thoracic (chest) and lumbar (lower back) injuries. In one embodiment, the subject has experienced a central nervous system injury, such as a brain injury, a stroke, or a traumatic spinal cord injury. For example, the central nervous system injury is associated with unwanted or abnormal muscle contractions or spasms, or the absence of normal muscle contractions.

In some embodiments, the subject has been diagnosed or identified as experiencing muscle cramps, spasms, dystonias, or fasciculations (e.g., unwanted or abnormal muscle cramps, spasms, dystonias, or fasciculations). Muscle cramps, spasms, dystonias, or fasciculations can also occur as a consequence of other diseases or disorders, such as diabetes (e.g., diabetic neuropathy), Addison's disease, peripheral artery disease, hypertension, alcoholism, liver cirrhosis, renal failure, hypothyroidism, neuromuscular diseases (e.g., amyotrophic lateral sclerosis, primary lateral sclerosis, progressive muscular atrophy, progressive bulbar palsy, pseudobulbar palsy, spinal muscular atrophy, progressive spinobulbar muscular atrophy (e.g., Kennedy's disease), or post-polio syndrome), and metabolic disorders (e.g., adrenoleukodystrophy, phenylketonuria, or Krabbe disease). Accordingly, the methods described herein are also useful for treating or evaluating subjects that have been diagnosed with the other diseases or disorders associated with muscle cramps described herein.

Muscle cramps, spasms, dystonias, or fasciculations can occur as a side effect of some drugs. Medications that can cause muscle cramps include: diuretics, oral contraceptives, blood pressure medications. The methods described herein can also be useful to treat or evaluate subjects that are prescribed or take medication that cause muscle cramps. Exemplary medications that can induce muscle cramps include, but are not limited to: diuretics, e.g., Lasix (furosemide), Microzide (hydrochlorothiazide); Alzheimer's disease medication, e.g., Aricept (donepezil); myasthenia gravis medication, e.g., Prostigmine (neostigmine); cardiovascular medication, e.g., Procardia (nifedipine); osteoporosis medication, e.g., Evista (raloxifene); asthma medication, e.g., Brethine (terbutaline), Proventil and Ventolin (albuterol); Parkinson's disease medication, e.g., Tasmar (tolcapone); cholesterol medication, e.g., statins such as Crestor (rosuvastatin), Lescol (fluvastatin), Lipitor (atorvastatin), Mevacor (lovastatin), Pravachol (pravastatin), or Zocor (simvastatin).

In an embodiment, the compositions and methods disclosed herein are suitable for treating or evaluating a subject that has an absence of a normal muscle contraction, such as a gait abnormality. Gait abnormalities are deviations from normal walking or unusual and uncontrollable walking patterns. Examples of gait abnormalities include propulsive gait, scissors gait, spastic gait, steppage gait, and waddling gait. For example, the gait abnormality is “foot drop,” in which the dropping of the forefoot happens due to muscular weakness, damage to nerves, or paralysis of muscles. Gait abnormalities are often associated with neuromuscular diseases or disorders.

### Connective Tissue Diseases

The compositions of the invention are also useful for treating connective tissue diseases. Examples of diseases include but are not limited to: degenerative joint disease (DJD), marfan syndrome, Ehlers-Danlos syndrome, osteogenesis imperfect, Stickler syndrome, Alport syndrome, congenital contractural arachnodactyly, psoriatic arthritis, systemic lupus erythematosus, rheumatoid arthritis, scleroderma, Sjögren's syndrome, and mixed connective tissue disease.

### Throat Conditions

The compositions of the invention can also treat throat disorders or throat injuries (e.g., from chemicals, cancer, surgery, or infection). Examples of throat disorders include, but are not limited to acid reflux, tonsillitis, pharyngitis, laryngospasm due to throat surgery, laryngitis, dysphagia, and spasmodic dysphonias.

### Sarcoidosis

The compositions of the invention are also useful for treating sarcoidosis. Sarcoidosis is a disease involving abnormal collections of inflammatory cells (granulomas) that can form as nodules in multiple organs. The granulomas are most often located in the lungs or its associated lymph nodes, but any organ can be affected. The compositions of the invention are useful in treating various types of sarcoidosis including but not limited to: annular sarcoidosis, erythrodermic sarcoidosis, ichthyosiform sarcoidosis, hypopigmented sarcoidosis, Löfgren syndrome, lupus pernio, morpheaform sarcoidosis, mucosal sarcoidosis, neurosarcoidosis, papular sarcoid, scar sarcoid, subcutaneous sarcoidosis, systemic sarcoidosis, and ulcerative sarcoidosis.

### Respiratory Conditions

The compositions of the invention may also useful for treating a respiratory condition or illness. Respiratory conditions involve the organs and/or tissues involved in respiration, including the lungs, trachea, bronchi, bronchioles, alveoli, pleura, pleural cavities. Without wishing to be bound by theory, activation of the TRPA1 and/or TRPV1 ion channels through administration of an ion channel activator (e.g., a TRPV1 channel activator, TRPA1 channel activator, ASIC channel activator, or combinations thereof) of the present invention may affect airway sensory nerve reactivity, thus leading to bronchodilation of airway smooth muscle. Exemplary respiratory conditions for which the composition of the present invention may be useful in treating include, but are not limited to, asthma, chronic obstructive pulmonary disease, bronchitis, emphysema, pneumonia, cystic fibrosis, pleural cavity diseases, influenza, or cold.

### Cough

The compositions of the present invention may also be useful for treating or reducing cough in a subject. Cough is a reflex that is often repetitive in nature and may aid in clearing the breathing passages from particles, irritants, secretions, and the like. Coughing may be voluntary or involuntary. Exemplary conditions related to cough include respiratory conditions (e.g., asthma, chronic obstructive pulmonary disease, bronchitis, emphysema, pneumonia, cystic fibrosis, pleural cavity diseases, influenza, or a cold), exposure to allergens or chemical irritants, or inflammation. In some embodiments, the composition of the present invention may be a cough suppressant.

### Anxiety Disorders

In some embodiments, the subject has been diagnosed with or identified as having an anxiety disorder. An anxiety disorder may be characterized as a condition involving the central nervous system and can cause feelings of fear, anxiety, and anguish in the subject. These conditions can result in unwanted or abnormal muscle cramps, spasms, dystonias, and fasciculations that may be treated by compositions of the disclosed invention. Exemplary anxiety disorders include generalized anxiety disorder, phobic disorders, panic disorders, agoraphobia, social anxiety disorder, obsessive-compulsive disorder, post-traumatic stress disorder, separation anxiety, and situational anxiety.

### Methods of Evaluating a Subject

The present invention provides various methods for evaluating a subject for the efficacy of a muscle cramp treatment or for treating an unwanted or abnormal muscle contraction, e.g., a muscle cramp, spasticity, dystonia, or fasciculation. In the methods described herein, a test muscle of a subject is electrically induced to have a test muscle cramp, and the electrical activity of the test muscle is recorded. In some embodiments, a test aliquot of a composition for treating muscle cramps, spasms, dystonias, or fasciculations is administered to the subject, and a second test muscle cramp is induced. The electrical activity of the second test cramp is also recorded. Comparison and analysis of the recordings of the first and second test cramp can indicate the efficacy of the test aliquot on reducing, alleviating, or preventing the cramp. Comparison and analysis of the recordings can also be used to classify subjects, or identify subjects for certain treatments for muscle cramps.

Alpha motor neurons project from the brainstem and the spinal cord and innervate the muscles. Stimulation of the alpha motor neurons results in transmittal of an electrical signal to the muscles, generated from the movement of ions across the cell membrane. The electrical stimulation from the motor neurons causes muscle movement or contraction, e.g., a muscle cramp or spasm. When the muscles are at rest, there is minimal or no electrical signal. Activity of the alpha motor neurons can be modulated by signaling from primary sensory neurons, which are activated by sensory input. Stimulation of non-taste primary sensory neurons with nerve endings in the mouth, esophagus and stomach, e.g., through activation of specific ion channels, can induce cause upregulation of inhibitory signals to the alpha motor neurons. Through this mechanism of interneuronal negative feedback, activation of primary sensory neurons inhibit or prevents alpha motor neuron firing via inhibitory signaling, and thereby inhibits muscle contractions of muscle cramps or spasms.

Electrical stimulation can elicit a muscle contraction that recapitulates a muscle cramp, spasm, dystonia, or fasciculation. In the methods described herein, electrical stimulation is used to induce a test muscle cramp. The electrical activity of the test muscle before, during, and after the test muscle cramp is detected, measured, and recorded. Analysis of the recording of the electrical activity of a muscle experiencing an electrically induced cramp can be useful for determining the efficacy of a treatment for alleviating a muscle cramp.

Electromyography is a technique for measuring and recording the electrical activity of a muscle during rest and movement. The instrument that detects and records the electric signal generated from a muscle is called an electromyograph. The recording of the electrical activity obtained by the electromyograph is known as an electromyogram (or myogram). The eletromyograph may comprise at least one recording electrode, at least one reference electrode, an amplifier unit, a device for converting the analog signals to digital signals, and a device for generating and displaying the recordings. Optionally, the instrument may also include at least one mechanism to detect additional biofeedback, such as body or skin temperature by a skin thermistor.

The electrical activity of a muscle, e.g., a test muscle, is recorded and detected by an electrode, or a lead. There are two main types of electrodes: surface electrodes and inserted electrodes. Surface electrodes are non-invasive and are placed on the skin. Inserted electrodes include needle and fine wire electrodes, and are inserted directly into the muscle tissue. In a preferred embodiment, the electrode used in the present invention is a surface electrode.

A recording electrode is preferably placed over a test muscle, and a reference electrode is placed nearby, e.g., within 2-6 inches of an active electrode. Preferably, the reference electrode is placed on a synergistic muscle. A synergistic muscle is a muscle that aids or participates in movement with the test muscle but does not cramp with the test muscle when electrically induced. In some preferred embodiments, a series or an array of multiple recording electrodes is used. For example, a linear array of 8 recording electrodes is applied on the test muscle, and optionally, a linear array of 4-8 recording electrodes is used on the synergistic muscle. In other embodiments, a grid array of recording electrodes is applied on the target muscle, e.g., a grid array of 6×5 electrodes is used.

In the methods described herein, electrical stimulus is applied to a test muscle to induce a test muscle contraction. The electrical stimulus is delivered by a stimulation electrode. The stimulation electrode is placed preferably directly on the skin over the test muscle. The electrical stimulus is defined by multiple parameters, such as stimulation frequency (Hertz or Hz), current intensity (milliamps or mA), pulse frequency (pulse per second or pps), and duration of time of stimulation. The electrical stimulus can be delivered by percutaneous electrical stimulation or surface electrical stimulation.

Preferred test muscles for electrically inducing a muscle cramp include the flexor hallucis brevis (FHB, or big toe flexor muscle) and gastrocnemius (calf muscle). Other target muscles may include the abductor hallucis (AH), the biceps brachii (biceps), the triceps surae (triceps), or the quadriceps femoris (quadriceps).

The appropriate stimulation frequency of electrical stimulation to induce a test muscle cramp may vary depending on the size or location of the muscle or the individual. For example, the electrical stimulation can be at least 1 Hz, at least 2 Hz, at least 3 Hz, at least 4 Hz, at least 5 Hz, at least 6 Hz, at least 7 Hz, at least 8 Hz, at least 9 Hz, at least 10 Hz, at least 11 Hz, at least 12 Hz, at least 13 Hz, at least 14 Hz, at least 15 Hz, at least 20 Hz, at least 25 Hz, at least 30 Hz, at least 35 Hz, at least 40 Hz, at least 50 Hz, at least 60 Hz, at least 70 Hz, at least 80 Hz, at least 90 Hz, or at least 100 Hz. In some preferred embodiments, the stimulation frequency for the FHB is 8 Hz, 10 Hz, 12 Hz, 14 Hz, or 18 Hz. In some embodiments, the stimulation frequency can vary over time. For example, the stimulation frequency increases over time, e.g., from 2 Hz to 24 Hz, increasing by 2 Hz increments.

The minimum frequency of the electrical stimulation capable of inducing a cramp has been termed the “threshold frequency.” Studies have shown that the threshold frequency for cramp induction is lower in cramp-prone subjects compared with subjects with no history of cramps. (Bertolasi et al., Ann. Neurol. 1993, 133:303-6; Miller and Knight, Muscle Nerve. 2009, 39:364-8; and Minetto et al., Muscle Nerve. 2009; 40:535-44). For example, Miller and Knight (Muscle Nerve. 2009; 39:364-8) found a threshold frequency for the flexor hallucis brevis muscle of approximately 15 Hz in subjects with a history of cramping and of approximately 25 Hz in individuals not prone to cramping.

The amplitude, or intensity of the current, of the electrical stimulus also may vary depending on the size or location of the muscle or the individual. A maximal current intensity refers to the current intensity required to achieve a plateau in the M-wave peak amplitude. The M-wave refers to the EMG signal detected. In a preferred embodiment, the current intensity is 30% supramaximal, or greater than, the maximal current intensity. In some embodiments, the maximal current intensity is determined for each individual subject prior to testing the subject. For example, the current intensity is 5 mA, 10 mA, 15 mA, 20 mA, 25 mA, 30 mA, 40 mA, 50 mA, or 60 mA.

The electrical stimulus can be applied as a series of pulses for a duration of time. For example, the stimulus may be applied as a series of at least 100 microsecond pulses, at least 120 microsecond pulses, at least 150 microsecond pulses, at least 180 microsecond pulses, at least 200 microsecond pulses, at least 300 microsecond pulses, at least 400 microsecond pulses, at least 500 microsecond pulses, or at least 600 microsecond pulses. The stimulus can be applied for 1 second, 2 seconds, 3 seconds, 4 seconds, 5 seconds, 6 seconds, 7 seconds, 8 seconds, 9 seconds, 10 seconds, or longer. Preferably for inducing cramps in the FHB, the stimulus is applied as a series of 180 microsecond pulses for 7 seconds.

In some embodiments, the parameters of electrical stimulation to be applied to the test muscle may be adjusted to decrease or increase the magnitude of the test muscle contraction to better recapitulate a muscle cramp, spasm, dystonia, or fasciculation. For example, the frequency or intensity of electrical current applied to the test muscle may vary depending on the type of test muscle contraction desired, e.g., the frequency of stimulation is increased to induce a test muscle cramp compared to a test muscle spasm.

In the methods described herein, the electrical activity of a target muscle is detected and recorded by a recording electrode before, during, and after an induced cramp. The electrical activity is recorded and displayed as a profile or electromyogram. Preferably, the profile contains the electrical activity before, during, and after the application of electrical stimulation to induce a muscle cramp. In some embodiments, the profile contains the electrical activity during the application of electrical stimulation and after the application of electrical stimulation. In some embodiments, the electrical activity is converted to root mean square (RMS) values and are displayed as a function of time. In the case where more than one recording electrode is utilized to measure the electrical activity of the target muscle, the profile contains the average electrical activity detected from all of the recording electrodes as a function of time.

The pattern of the recorded electrical activity or signal can indicate the presence or absence of an induced muscle cramp. Indication of an induced muscle cramp includes involuntary electrical signal of the stimulated muscle after cessation of the electrical stimulation, preferably with concurrent absence of electrical signal of the synergistic muscle. Alternatively, a signal amplitude greater than 2 or 3 standard deviations above the 1 second baseline signal amplitude of either the target muscle prior to stimulation or the synergistic muscle after stimulation indicates an induced cramp. The induced muscle cramp can last for at least 5 seconds, at least 10 seconds, at least 15 seconds, at least 20 seconds, at least 25 seconds, at least 30 seconds, at least 35 seconds, at least 40 seconds, at least 45 seconds, at least 50 seconds, at least 55 seconds, at least 1 minute, at least 2 minutes, at least 3 minutes, at least 4 minutes, or at least 5 minutes.

In some embodiments, a first muscle cramp is electrically-induced in a subject prior to administration of a composition for treating a muscle cramp, and after a rest period, a second muscle cramp is electrically-induced in a subject after administration of the composition. The profile obtained for the first induced cramp before administration of the composition is referred to as the reference profile. The profile obtained for the second induced cramp after administration of the composition is referred to as the treatment profile. Comparison of the treatment profile and the reference profile can be used to select subjects for treatment with the composition or to identify subjects that will be responsive to the treatment with the composition. Specifically, the properties of the induced cramp in the reference and treatment profiles are compared to determine whether the composition administered reduced or prevented the second induced cramp.

Alternatively, a value can be determined from the reference profile, e.g., a reference value, and a value can be determined from the treatment profile, e.g., a treatment value. The reference value and the treatment value can be compared to determine a reduction or prevention of a cramp. In one embodiment, the values determined from the reference and/or treatment profile represent the parameters of muscle cramp. A decrease in the treatment value compared to the reference value indicates that that the test aliquot administered is effective at alleviating or preventing a muscle cramp.

The parameters of a muscle cramp can be compared for determining the efficacy of a treatment for alleviating a cramp, e.g., by a reduction of a muscle cramp parameter or prevention of a cramp. Parameters of muscle cramps that can be determined from the electromyogram include the area under the curve, the peak amplitude, the duration of the cramp, and a change in threshold frequency used to elicit a test muscle cramp.

An area under the curve value can be calculated for the reference and treatment profile using standard methods known in the art. A decrease or absence of an area under the curve value in the treatment profile compared to the reference profile indicates that an electrically induced cramp has been reduced or prevented. For example, the area under the curve in the treatment profile may be at least 1%, 5%, 10%, 15,%, 20%, 25%, 30%, 35%, or 50% decreased compared to the area under the curve in the reference profile.

The peak amplitude after cessation of the electrical stimulus can be compared between the reference and treatment profiles. A reduction in or absence of the peak amplitude in a treatment profile compared to a reference profile indicates that an electrically induced cramp has been reduced or prevented. For example, the peak amplitude in a treatment profile may be at least 1%, 5%, 10%, 15,%, 20%, 25%, 30%, 35%, or 50% decreased compared to the peak amplitude of the reference profile.

The duration of the test cramp can be compared between the reference and treatment profile. A decrease in or absence of the duration of the cramp indicates the reduction or prevention of the electrically induced cramp. For example, the duration of the cramp may be at least 1%, 5%, 10%, 15,%, 20%, 25%, 30%, 35%, or 50% decreased compared to the duration of the cramp in the reference profile.

Threshold frequency refers to the minimum frequency of electrical stimulation required to elicit a cramp. In an embodiment, a change in the threshold frequency required to elicit a test muscle cramp indicates the efficacy of a treatment for alleviating a cramp. For example, a change in the threshold frequency may be at least 1%, 5%, 10%, 15,%, 20%, 25%, 30%, 35%, or 50%, 100%, 200% increased compared to the threshold frequency required before treatment, e.g., in the reference profile. For example, a change in the threshold frequency may be at least 1%, 5%, 10%, 15,%, 20%, 25%, 30%, 35%, or 50%, decreased compared to the threshold frequency required before treatment, e.g., in the reference profile.

In certain embodiments, the rest period between the first test cramp and the second test cramp is at least 1 minute, 2 minutes, 5 minutes, 10 minutes, 15 minutes, 20 minutes, 25 minutes, 30 minutes, 35 minutes, 40 minutes, 45 minutes, 50 minutes, 55 minutes, 60 minutes, 90 minutes, 2 hours, 3 hours, 4 hours, 5 hours, 6 hours, 7 hours, 8 hours, 9 hours, 10 hours, 11 hours, 12 hours, 16 hours, 20 hours, or 24 hours. In certain embodiments, the electrical stimulation is re-applied to induce a second muscle cramp at least 1 minute, 2 minutes, 5 minutes, 10 minutes, 15 minutes, 20 minutes, 25 minutes, 30 minutes, 35 minutes, 40 minutes, 45 minutes, 50 minutes, 55 minutes, 60 minutes, 90 minutes, 2 hours, 3 hours, 4 hours, 5 hours, 6 hours, 7 hours, 8 hours, 9 hours, 10 hours, 11 hours, 12 hours, 16 hours, 20 hours, or 24 hours after administration of the composition for treating muscle cramps.

In one embodiment, the test comprises determining that a cramp can be induced in a subject by application of stimulus. For example, the stimulus is percutaneous electrical stimulation or surface electrical stimulation. In some embodiments, the magnitude of the parameters of the induced muscle cramp, e.g., as determined from an EMG profile, identifies or classifies subjects with respect to selection of treatment regimens, or predicts the response of a subject to a specific treatment regimen.

### Combination Therapies

In certain embodiments, additional therapeutic agent(s) may be administered with compositions of the present invention for, e.g., the treatment of peripheral nervous system conditions (e.g., peripheral neuropathy), central nervous system conditions, muscle conditions and disorders (e.g., fibromyalgia, muscle spasms and cramps (e.g., nocturnal cramps), painful muscle contractions (e.g., a muscle contraction of the head or neck), neuromuscular disorders (e.g., motor neuron disease) or dystonia (e.g., cervical dystonia, blepharospasm, back spasms, or leg cramps due to spinal stenosis)), connective tissue diseases (e.g., degenerative joint disease), throat conditions (e.g., dysphagia or spasmodic dysphonias), tactile sensitivity, electrolyte imbalance and/or vitamin deficiency, respiratory conditions (e.g., asthma), cough, and sarcoidosis. In one embodiment, the candidate therapeutic agents are agents already known in the art for use for other conditions or disorders, e.g., neuromuscular therapeutic agents. When combination therapy is employed, the additional therapeutic agent(s) can be administered as a separate formulation or may be combined with any of the compositions described herein.

For example, any of the compositions described herein can be used for the treatment of nocturnal (or night) cramps. In some embodiments, the compositions can be used in combination with a sleep aid. Sleep aids that can be used in combination with the compositions and methods described herein include: antihistamines (e.g., diphenhydramine and doxylamine); benzodiazepines (e.g., estazolam (ProSom), flurazepam (Dalmane), quazepam (Doral), temazepam (Restoril), and triazolam (Halcion)); non-benzodiazepine sedative hypnotics (e.g., eszopiclone (Lunesta), zalepon (Sonata), and zolpidem (Ambien)); and melatonin receptor agonist hypnotics (e.g., ramelteon (Rozerem). Still other sleep aids that can be used in combination with the compositions and methods described herein include: chamomile, valerian root, kava kava, lemon balm, passionflower, lavender, St. John's Wort, melatonin, tryptophan (e.g., L-tryptophan), 5-hydroxytryptophan (5-HTP), catnip, hops, rhodiola, oatstraw, lavender, GABA, L-theanine, linden, ginseng (e.g., Siberian ginseng), honey, nutmeg, mugwort, butterbur, rauwolfia, taumelloolch, American hellebore, quassia, tulip tree, brewer's yeast, inositol, skullcap, phosphatidylserine, calcium, magnesium, vitamin B6, vitamin B12, and pantothenic acid (B5).

In another embodiment, any of the compositions described herein can be used for the treatment of painful muscle contraction of the head or neck as in tension, cluster or migraine headache. In some embodiments, the compositions can be used with analgesics, including aspirin, ibruprofen, acetaminophen, or naproxen; with triptans including sumatriptan, rizatriptan, naratriptan; with mild sedatives including butalbital; with anti-depressants including amitriptyline; with dihydroergotamine mesylate; or with ketorolac.

Any of the compositions described herein can be also be used for the treatment of focal dystonia. In some embodiments, the compositions can be used with botulinum toxin; with anticholinergic agents including trihexyphenidyl and benztropine; with GABAergic agents including benzodiazepines; and with dopaminergic agents including tetrabenazine and levodopa.

In further embodiments, the compositions described herein can be also be used for the treatment of muscle claudication pain due to inactivity or restriction as seen in “economy class syndrome”, paralysis, peripheral artery disease or immobilization. In some embodiments, the compositions can be used with cilostazol or with pentoxifylline. The compositions described herein can be also be used for the treatment of sarcoidosis. In some embodiments, the compositions can be used with non-steroidal anti-inflammatory drugs (NSAIDs) including ibuprofen and aspirin; with corticosteroids, including prednisone and prednisolone; and with steroid-sparing agents, including azathioprine, methotrexate, mycophenolic acid, and leflunomide.

In other embodiments, any of the compositions described herein can also be used in combination with a treatment for pain or a disorder relating to the oral cavity, such as oral lesions, canker sores, cold sores, thrush, gingivitis, leukoplakia, halitosis, or dry mouth. In some embodiments, the composition can be used with or antibacterial or antiviral agents to treat or prevent tooth decay or carries.

In other embodiments, any of the compositions described herein can also be used in combination with a treatment for pain or a disorder relating to the stomach or gastrointestinal tract, such as indigestion, heartburn, colitis, irritable bowel syndrome, constipation, diarrhea, lactose intolerance, gastroesophageal reflux disease, ulcers, nausea, or stomach cramps. In some embodiments, the compositions can be used with antacids (e.g., simethicone, magaldrate, aluminum salts, calcium salts, sodium salts, magnesium salts, alginic acid) laxatives, H₂ antagonists (e.g., ranitidine, famotidine, nizatidine, cimetidine) or proton pump inhibitors (e.g., omeprazole, lansoprazole, esomeprazole, dexlansoprazole, rabeprazole, or pentoprazole), and antidiarrheals (e.g., bismuth subsalicylate).

In other embodiments, any of the compositions described herein can be also be used for the treatment of disease, disorder or injury to the peripheral nervous system such as cramp fasciculation syndrome, peripheral neuropathy, carpal tunnel syndrome or EBV. In some embodiments, the compositions can be used to treat cramp fasciculation syndrome with β-blockers; analgesics including ibuprofen and acetaminophen; magnesium; or carbamazepine. In some embodiments, the compositions can be used to treat peripheral neuropathy with tricyclic antidepressants, including amitriptyline; with antiepileptic therapies including gabapentin and sodium valproate; with synthetic cannabinoids including nabilone; with pregabalin; or with serotonin-norepinephrine reuptake inhibitors (SNRIs), including duloxetine. In some embodiments, the compositions can be used to treat carpal tunnel syndrome with corticosteroids.

### EXAMPLES

### General Procedures

### TRP-Stim Solution

The solution (“TRP-Stim”) administered to the volunteers contains: a base of a 1:1 mixture of water and light karo syrup (for increased viscosity); 0.075% of a capsicum preparation intended for human use (Clearcap Super Soluble Caspsicum, alsec Inc.); 1% of a cinnamon volatile oil intended for human consumption (Aquaresin Cinnamon, Kalsec Inc); and 1.5% of a ginger oleoresin intended for human use (Aquaeresin Ginger, Kalsec Inc).

### Electromyography (EMG) Measurements of Cramps

Methods for placing stimulating electrodes on the flexor hallucis brevis (FHB) or gastrocnemius muscles followed the procedures described by Minetto et al., Muscle Nerve, 40: 535-544, 2009, the contents of which are incorporated herein by reference in its entirety. The active stimulation electrode (cathode) is a 1.25″ circular mesh-backed silver patch electrode (Bio-Flex manufactured by Lead-Lok) and is placed so as to produce contraction of the FHB with minimal stimulation amplitude. The stimulation reference electrode is a 2″ square patch electrode (Bio-Flex manufactured by Lead-Lok) is placed on the opposite side of the foot, e.g., under the lateral malleous. Cramping of the FHB is induced as described by Minetto et al. (ibid.) using a battery-powered electrical muscle stimulator (EMS-7500, Current Solutions LLC) to deliver pulses. A series of 180 microsecond biphasic square pulses of voltages is applied at various frequencies to stimulate the muscle. First, using slow (2 Hz) stimulation, the amplitude is adjusted to ˜30% more than the threshold amplitude for eliciting strong contraction of the muscle. The muscle is then stimulated by a train of 180 microsecond pulses of this amplitude delivered for 7 seconds at various frequencies. The stimulation delivered by the stimulator includes “ramp up” and “ramp down” periods of 1 second preceding and following the main 7-second stimulation period during which the amplitude of the pulses is ramped up or down to and from the final value.

It has been previously shown that susceptibility to cramping of the FHB using similar electrical stimulation protocols is highly reproducible within each subject (Minetto et al., Muscle Nerve 2008, 37:90-100, 2008) and is correlated with susceptibility to “ordinary muscle cramps” (Miller et al., Muscle Nerve 2009, 39:364-368).

Cramping is quantified by making EMG recordings from the belly of the FHB. Two external EMG recording electrodes (Vermed SilveRest) are placed along the belly of the FHB. The differential voltage relative to a third ground electrode placed at the ankle is amplified, digitized, and saved to computer using a 1-330-C2+EMG unit with PhysioLab software (J&J Engineering, Poulsbo, Wash.). The raw wide-band EMG signal (10-400 Hz) is processed by being rectified and integrated to provide the area under the curve (RMS). The duration of cramp is quantified by the time required for the RMS EMG to return to an amplitude of 3 standard deviations above the baseline value. This will correlate well with duration of the cramp as observed by the return to the toe to resting position.

Recordings of cramps in calf muscles (medial gastrocnemius) are made using similar procedures, with placement of stimulation and recording electrodes following that by Minetto et al., Muscle Nerve 2009, 40:535-544). The amplitude of stimulation by a single 180 microsecond biphasic square pulses is adjusted to be ˜30% of the amplitude required for maximal contraction of the muscle. After a short period of slow stimulation (2 Hz), the frequency of stimulation is ramped up to 22-24 Hz over ˜5 seconds and is held at this frequency for an additional 5 seconds before terminating the stimulation. This protocol reliably induces cramping of 30-90 seconds.

### Assay of Activation of Rat Sensory Neurons

Methods to monitor activation of primary sensory neurons isolated from the trigeminal ganglion of rats follow those published by Park et al. (J Biol Chem. 2006, 281:17304-17311). Cells isolated from rat trigeminal ganglia are loaded with the fluorescent calcium indicator Fura-2AM (Fura-2-acetoxymethyl ester), and increases in intracellular calcium reflecting activation of the neurons are measured as an increase in Fura-2 fluorescence as measured by digital video micro-fluorometry with an intensified CCD camera. The same capsicum extract, cinnamon extract, and ginger extract used in the TRP-Stim beverage are applied to the neurons after dilution in balanced salt solution (in mM: 145 NaCl, 5 KCl, 2 CaCl₂, 1 MgCl₂, 10 HEPES, and 10 glucose) for perfusion of the neurons. Caspicum extract is applied at a dilution of 1/800,000, cinnamon extract at a dilution of 1/5,000, and ginger extract at a dilution of 1/12,000. In some experiments the calcium ionophore ionomycin is added following the tests with extracts to produce a large entry of calcium as an index of the maximal possible signal, illustrating the strength of activation by the heavily diluted extracts.

### Example 1

### Activation of Rat Sensory Neurons by Capsicum, Cinnamon, and Ginger Extracts

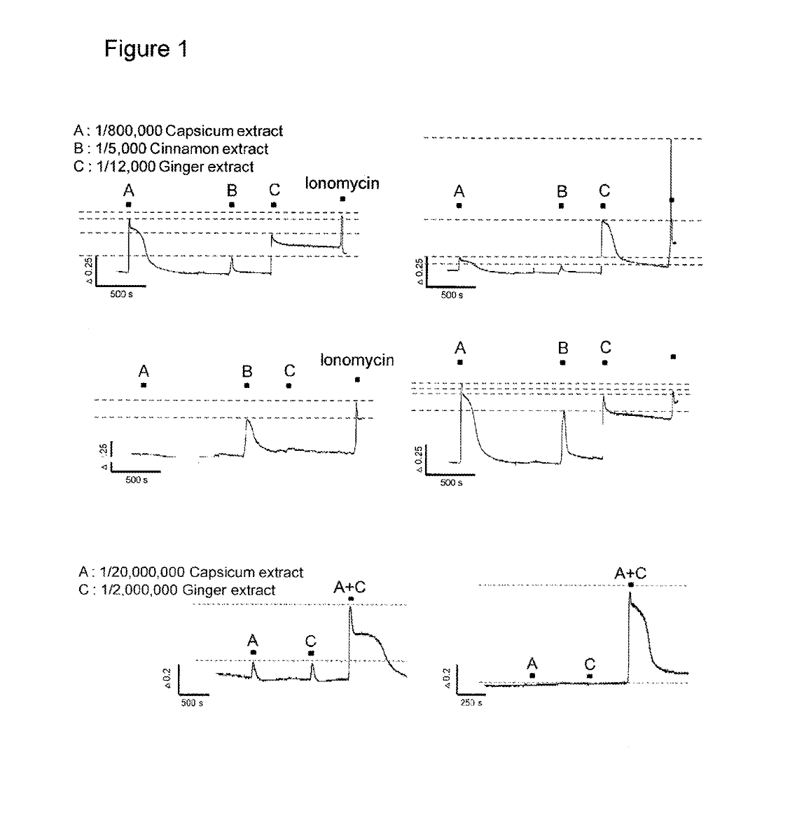

FIG. 1 shows graphs from six sensory neurons isolated from the trigeminal ganglia of rats, illustrating their activation by the capsicum, cinnamon, and ginger extracts that are used in the human experiments. Activation is quantified as an increase in intracellular free calcium, monitored by a fluorescent calcium indicator. Extracts are diluted into normal extracellular saline (Tyrode's solution) and are tested at lower concentrations than used in the beverage, taking account that concentrations present at nerve endings in mouth, esophagus, or stomach are expected to be lower than the beverage as a result of dilution into mucosa and interstitial fluid. All three extracts are capable of activating individual neurons when applied at concentrations 50-fold to 15,000-fold lower than used in the beverage. Each trace shows a record from a different neuron, illustrating that some neurons could be activated by each of the extracts and that the strength of activation by each extract varied among particular neurons. These records illustrate that each agent is capable of acting alone to activate some neurons and that a combination of agents can produce stronger activation of a larger fraction of neurons. Further, the bottom two records show that there can be strongly synergistic activation of neurons by the capsicum extract and the ginger extract when applied in combination.

### Example 2

### Effect of TRP-Stim Administration to Human Subjects

The in vitro data of Example 1 show that each individual component of the TRP-Stim solution by itself is capable of activating sensory neurons. Consistent with this, human experiments show the efficacy of a beverage with capsicum alone (ClearCap capsicum at 1/2000 dilution) to inhibit cramping, achieved within 5 minutes.

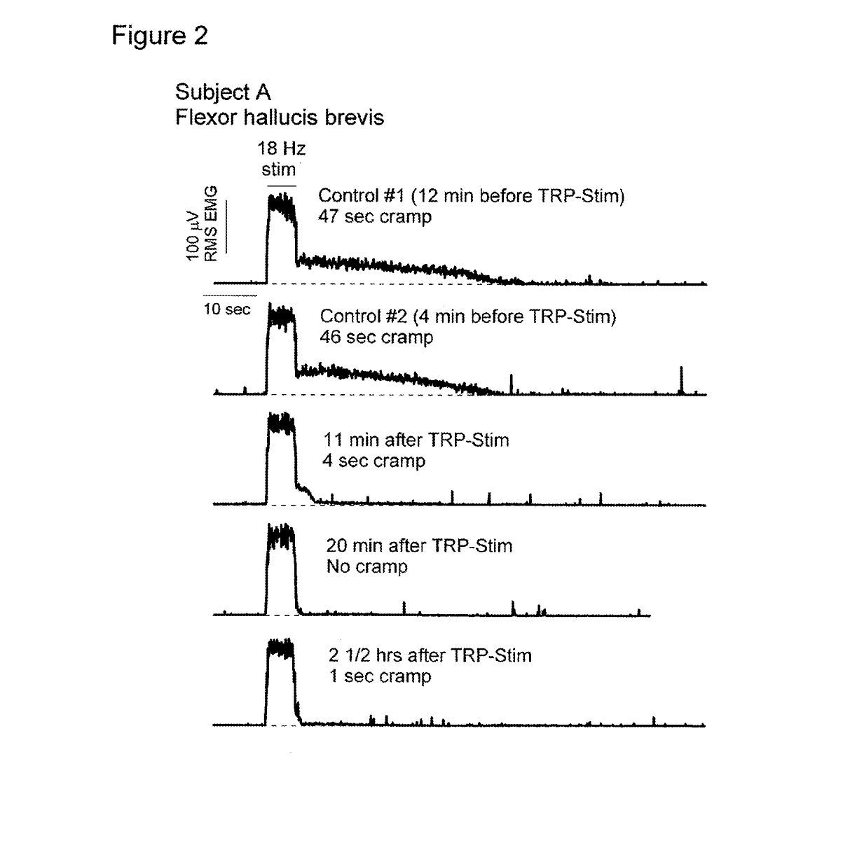

The following experiments, illustrated by FIGS. 2-8, show cramp relief by the administration of a uniform beverage composition designed for maximal TRP stimulation containing capsicum, cinnamon extract, and ginger extract, and where the physiological effects were monitored by EMG recording. These experiments demonstrate the utility of evaluating subjects using EMG for determining the efficacy of a composition for alleviating an electrically induced cramp. The methods demonstrated in this example can also be used in subjects that have been diagnosed or identified with a disorder associated with muscle cramps, e.g., a disorder disclosed herein, such as night cramps or a neuromuscular disorder, e.g., multiple sclerosis, spinal cord spasticity, and dystonia.

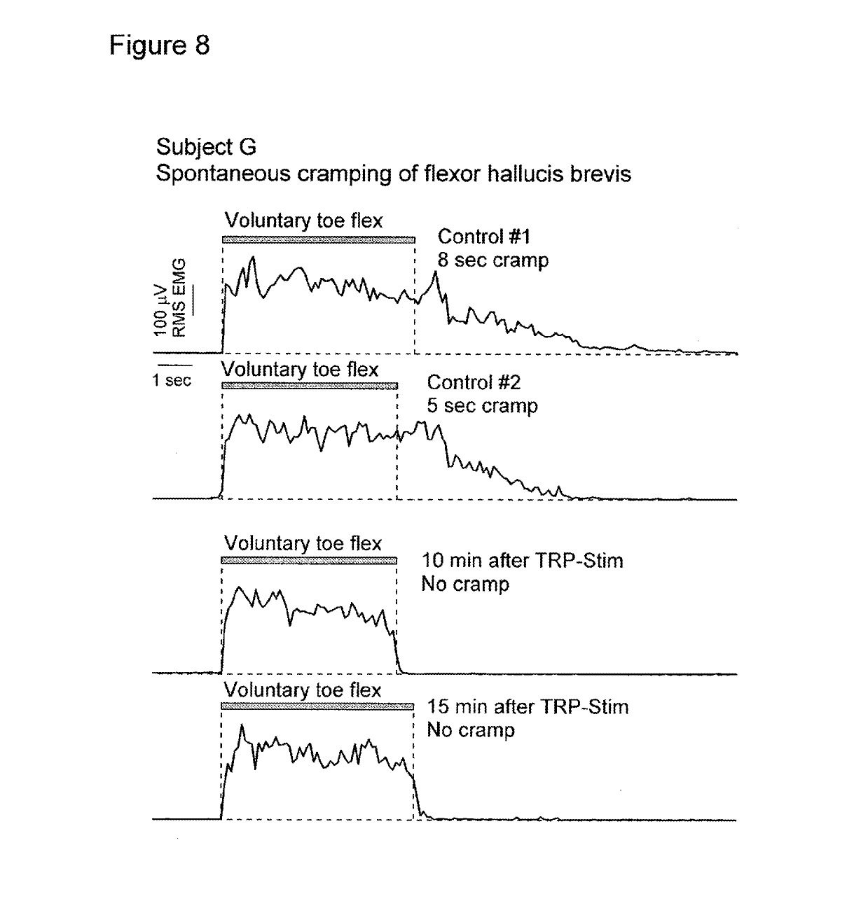

FIGS. 2-8 are graphs of EMG recordings of muscle contractions in seven human volunteers (four females and three males) that show the efficacy in preventing and treating cramps of ingesting 50 mL of a solution designed to stimulate TRP VI and TRPA1 receptors in the mouth, esophagus, and stomach. Muscle cramps are induced by brief stimulation of toe or calf muscles (FIGS. 2-7) or occurred spontaneously (FIG. 8). After recording cramping in control, subjects drink 50 mL of the TRP-Stim solution containing capsaicin and capsaicinoids (TRPV1 agonists), cinnamaldehyde (TRPA1 agonist), and gingerols (TRPA1 and TRPV1 agonists). After ingestion of the solution, subjects are tested for muscle cramping using the same procedures as in control at times ranging from 4 minutes to 11 hours after ingestion.

Eight human volunteers are tested using the TRP-Stim beverage. Seven of the eight show an abolition or dramatic reduction in cramping following ingestion of the beverage (FIGS. 2-8). The effect is typically complete within 4-15 minutes and lasted for 2½ to 4 hours in different subjects. An eighth subject showed cramping of the FHB that is not dramatically affected by the TRP-Stim beverage. The cramping in this subject is of much lower EMG amplitude that the other subjects and appeared to involve repetitive contraction of only a few motor units.

FIG. 2 is a graph showing the effect of the TRP-Stim beverage on cramping of the flexor hallucis brevis of Subject A. Under control conditions, cramping is reliably induced by stimulating the muscle using an electrical muscle stimulator (EMS-7500, Current Solutions LLC) placed with external electrodes for FHB stimulation. Muscle activity is recorded using external electrodes placed over the belly of the muscle attached to an EMG amplifier (J&J Engineering I-330C2+). In control, stimulation using 180 microsecond biphasic pulses delivered at 18 Hz for 5 seconds reliably and reproducibly produce cramping of the muscle, which is evident by EMG activity continuing after the cessation of stimulation. After ingestion of the TRP-Stim beverage, cramping is very brief after 11 minutes and essentially absent at tests at 20 minutes and 2½ hours after ingestion.

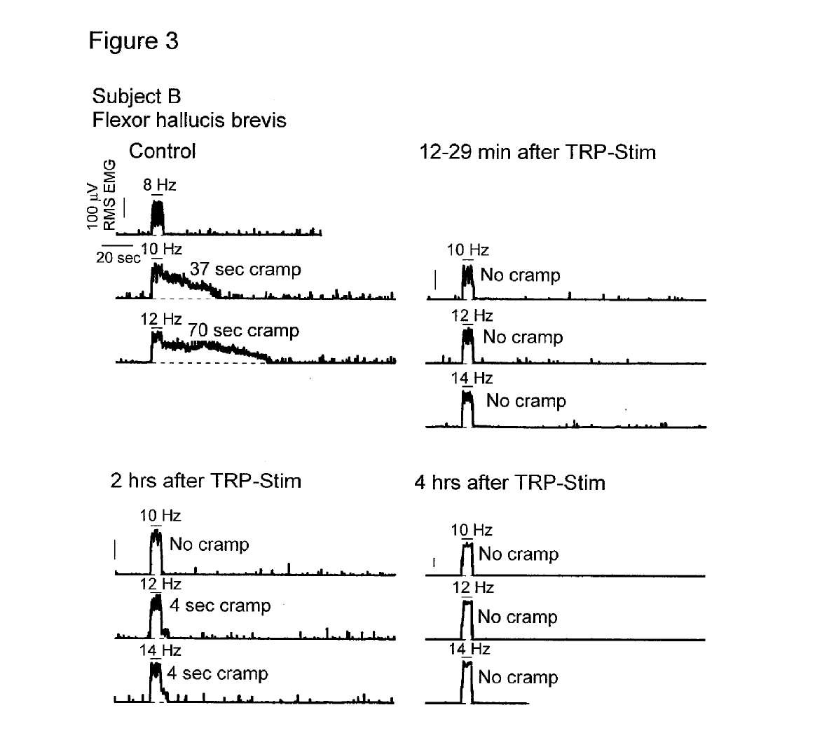

FIG. 3 is a graph showing the effect of the TRP-Stim beverage on cramping of the flexor hallucis brevis of a second subject. Under control conditions, cramping is induced by stimulation at 10 Hz for 5 seconds (180 microsecond pulses, amplitude set to ˜30% higher than threshold for muscle contraction), and a longer cramp is induced by increasing the frequency to 12 Hz. In recordings beginning 12 minutes after ingestion of the TRP-Stim beverage, stimulation at 10 Hz or 12 Hz produced essentially no cramping, and increasing the frequency of stimulation to 14 Hz also did not induce cramping. The dramatic reduction in cramping was still present 4 hours later in this subject.

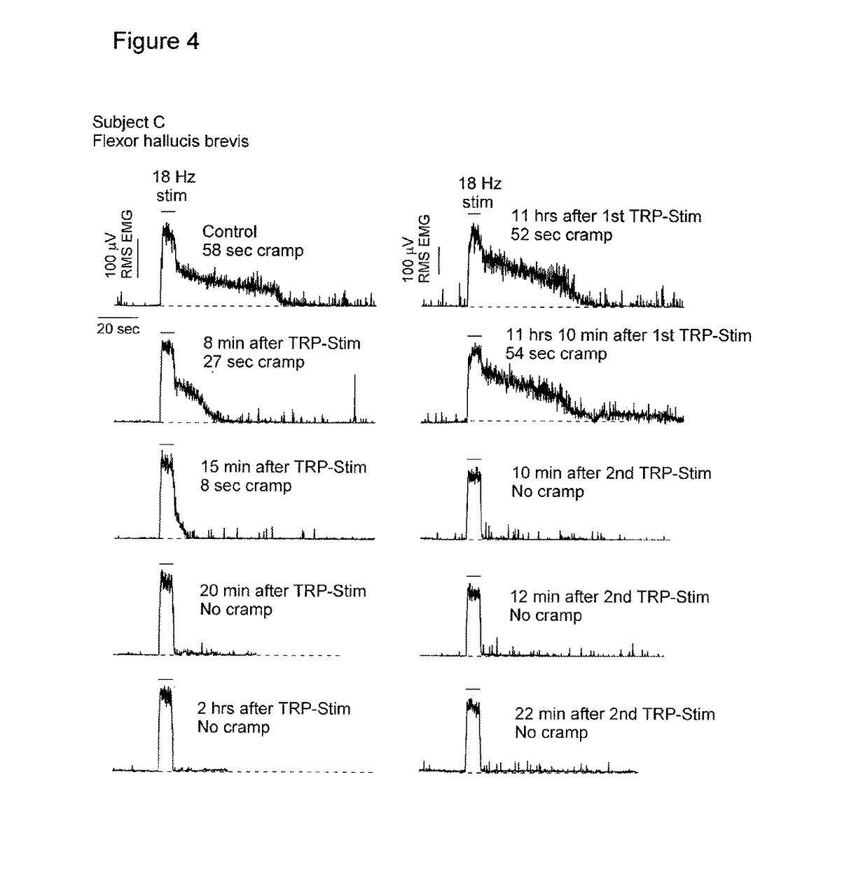

FIG. 4 is a graph showing the effect of the TRP-Stim beverage on cramping of the flexor hallucis brevis of a third subject tested over longer times. Under control conditions, a cramp lasting 58 seconds is induced by stimulation at 18 Hz for 5 seconds (180 microsecond pulses, amplitude set to ˜30% higher than threshold for muscle contraction). After ingestion of the TRP-Stim beverage, the duration of the cramp is reduced to 27 seconds after 8 minutes and to 8 seconds after 15 minutes. Cramping is abolished after 20 minutes and in a test after 2 hours. In tests 11 hours after ingestion, reliable cramping returns. After the subject again drinks 50 mL of the TRP-Stim beverage, cramping is completely abolished in tests beginning after 10 minutes.

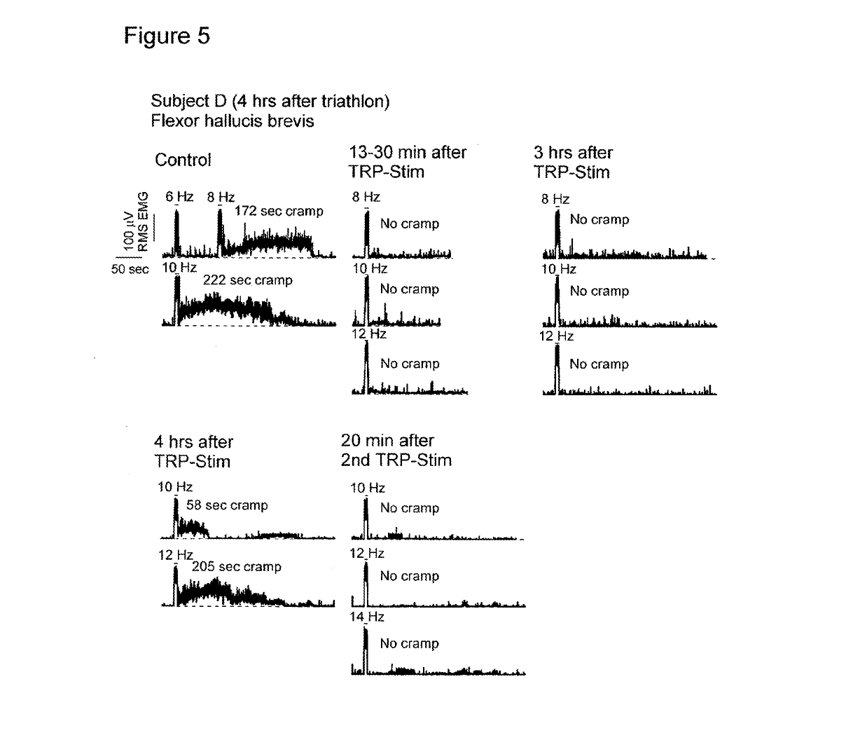

FIG. 5 is a graph showing the effect of the TRP-Stim beverage on cramping of the flexor hallucis brevis of a fourth subject. This subject engages in strenuous exercise (triathlon) four hours earlier and experiences muscle twitchiness. This subject has an unusually low frequency threshold (8 Hz) for induction of cramping in the FHB muscle, and the resulting cramps are unusually long (172 seconds after 8 Hz stimulation and 222 seconds after 10 Hz stimulation). Cramping is completely gone in tests starting 13 minutes after ingestion of the TRP-Stim beverage, even when increasing the stimulation frequency to 12 Hz. Cramping is still abolished 3 hours later. After 4 hours, cramping returns with an increased frequency threshold (10 Hz) and shorter cramps than in control. After the subject again drinks 50 mL of the TRP-Stim beverage, cramping is again completely abolished.

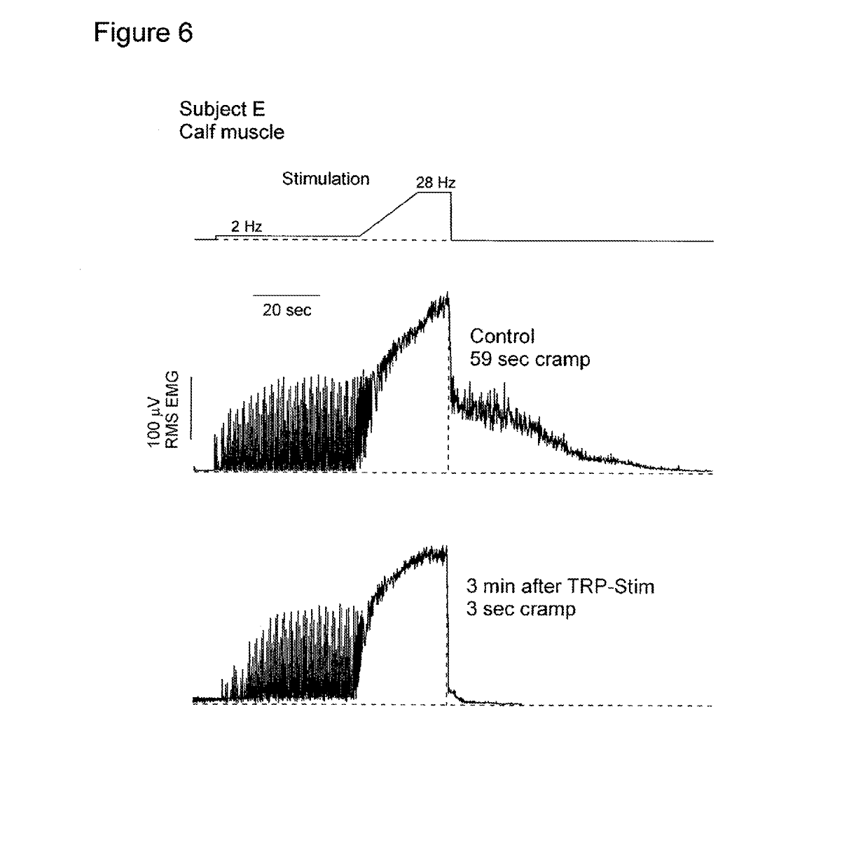

FIG. 6 is a graph showing the effect of the TRP-Stim beverage on cramping of the gastrocnemius (calf) muscle of a fifth subject. The muscle is stimulated by a protocol ramping the frequency of stimulation from 2 Hz to 28 Hz (180 microsecond pulses, amplitude set to ˜30% higher than threshold for muscle contraction). After cessation of stimulation, the muscle went into a prolonged cramp lasting 59 seconds. In a test 3 minutes after ingestion of 50 mL of TRP-Stim, cramping is abolished.

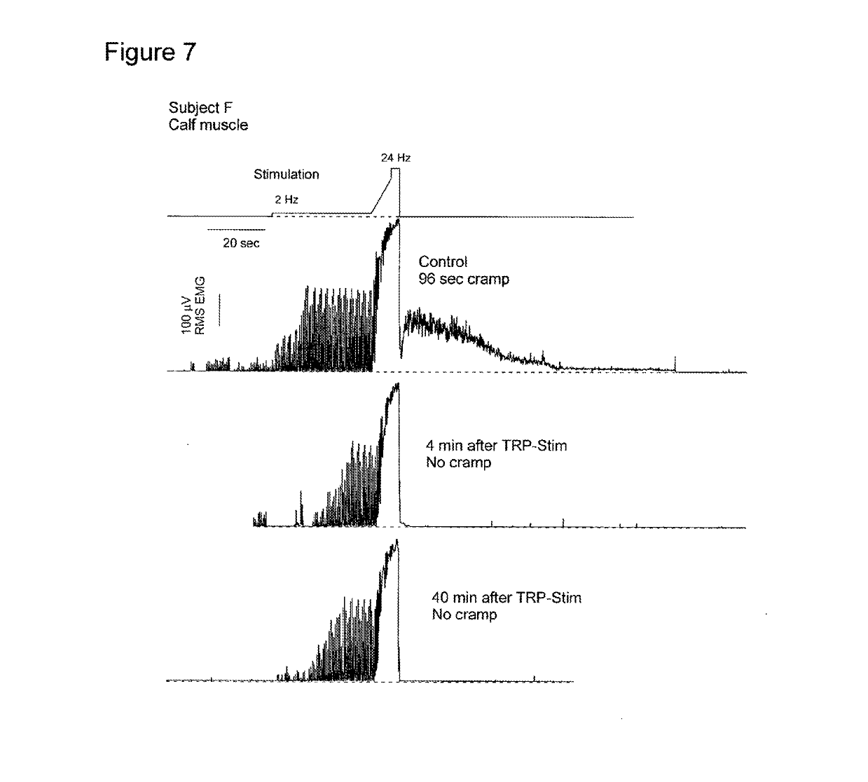

FIG. 7 is a graph showing the effect of the TRP-Stim beverage on cramping of the gastrocnemius (calf) muscle of a sixth subject. The muscle is stimulated by a protocol ramping the frequency of stimulation from 2 Hz to 24 Hz (180 microsecond pulses, amplitude set to ˜30% higher than threshold for muscle contraction). After cessation of stimulation, the muscle went into a prolonged cramp lasting 96 seconds. In a test 4 minutes after ingestion of 50 mL of TRP-Stim, cramping is abolished. Cramping is still abolished in a test conducted 40 minutes later.

FIG. 8 is a graph showing the effect of the TRP-Stim beverage on cramping of an FHB muscle in a seventh subject, who experiences spontaneous cramping induced by pointing her toe. In control conditions, voluntary toe flexes lasting ˜5 seconds reliably produces cramping of the FHB lasting 5-8 seconds in different trials. Ten minutes after the subject ingests 50 mL of the TRP-Stim beverage, cramping is abolished.

The methods described in this example can be used to evaluate subjects with disorders associated with muscle cramps, for example, subjects that experience night cramps or have been diagnosed or identified as having a neuromuscular disease, e.g., multiple sclerosis, spinal cord spasticity, or dystonias. For example, the methods described in this example can be used to assess the efficacy of any of the compositions described herein for alleviation of an electrically-induced muscle cramp in a subject diagnosed or identified as having a disorder associated with muscle cramps. Also, the methods described in this example can also be used to evaluating and classifying a subject diagnosed or having a disorder associated with muscle cramps.

### Other Embodiments

From the foregoing description, it is apparent that variations and modifications may be made to the invention described herein to adopt it to various usages and conditions. Such embodiments are also within the scope of the following claims.

All publications, patent applications, and patents mentioned in this specification are herein incorporated by reference to the same extent as if each independent publication, patent application, or patent was specifically and individually indicated to be incorporated by reference.

What is claimed is:

1. A composition formulated for oral administration, said composition comprising an effective amount of an ion channel activator and a pharmaceutically acceptable excipient, wherein said composition is a liquid or solid, and wherein said composition is formulated for delayed release of said ion channel activator.

2. The composition of claim 1, wherein said composition comprises a plurality of ion channel activators and a pharmaceutically acceptable excipient.

3. The composition of claims 1 and 2, wherein said composition comprises two ion channel activators and a pharmaceutically acceptable excipient.

4. The composition of any one of claims 1-3, wherein said composition comprises an ion channel activator and a plurality of pharmaceutically acceptable excipients.

5. The composition of any one of claims 1-4, wherein said pharmaceutically acceptable excipient comprises an agent for delayed release of an ion channel activator such that, when orally administered to a subject, the ion channel activator is not substantially released in the stomach of said subject.

6. The composition of claim 5, wherein the agent for delayed release is selected from the group consisting of: hydroxypropyl cellulose, hydroxypropyl methyl cellulose, methyl cellulose, carboxymethyl cellulose, and mixtures thereof.

7. The composition of any one of claims 5-76, wherein said pharmaceutically acceptable excipient comprises a coating.

8. The composition of claim 7, wherein said coating is selected from the group consisting of: enteric coatings, sugar coatings, and polymeric coatings.

9. The composition of any one of claims 1-8, wherein said ion channel activator is embedded in biodegradable microparticles or nanoparticles for sustained release.

10. The composition of any one of claims 1-9, wherein said composition further comprises a formulation base.

11. The composition of claim 10, wherein said formulation base comprises an oil and a lipophilic additive.

12. The composition of claim 12, wherein said oil is selected from the group consisting of: vegetable oil, mineral oil, soya oil, sunflower oil, corn oil, olive oil, nut oil, and liquid paraffin.

13. The composition of claim 12, wherein said lipophilic additive is selected from the group consisting of: polyethylene glycol, fatty acid mono-, di-, or triglycerides, palmitic acid, stearic acid, behenic acid, polyethylene glycol fatty acid esters, candelilla wax, carnauba wax, polyethylene oxide wax, and petroleum wax.

14. The composition of any one of claims 1-13, wherein the composition further comprises a coloring agent, a dissolving agent, a flavoring agent, a sweetener, a viscosity modifier, an electrolyte, a vitamin, a mineral, an antioxidant, or a preservative.

15. The composition of any one of claims 1-14, wherein said composition is formulated as a liquid.

16. The composition of claim 15, wherein said liquid is selected from the group consisting of emulsions, microemulsions, solutions, suspensions, syrups, linctuses, drops, sprays, and elixirs.

17. The composition of any one of claims 1-14, wherein said composition is formulated as a solid.

18. The composition of claim 17, wherein said solid is selected from the group consisting of tablets, capsules, powders, crystals, pastes, gels, lozenges, gums, candies, chews, foodstuffs, dissolving strips, films, and semi-solid formulations.

19. The composition of claim 18, wherein said capsule is a hard or soft capsule.

20. The composition of any of claims 1-19, wherein the ion channel activator comprises a TRPV1 channel activator, a TRPA1 channel activator, an ASIC channel activator, or a combination thereof.

21. The composition of any one of claim 20, wherein said TRPV1 channel activator is a capsaicinoid, a capsinoid, oleoylethanolamide, N-oleoyldopamine, 3-methyl-N-oleoyldopamine, oleamide, capsiate, a 1-monoacylglycerol having C18 and C20 unsaturated and C8-C12 saturated fatty acid, a 2-monoacylglycerol having C18 and C20 unsaturated fatty acids, miogadial, miogatrial, polygodial, a terpenoid with an alpha,beta-unsaturated 1,4-dialdehyde moiety, sanshool, evodiamine, acesulfame-K, cyclamate, CuSO₄, ZnSO₄, FeSO₄, arvanil, anandamide, N-arachidonoyl-dopamine, flufenamic acid dopamide, a dopamine amide of fenamic acid, 4-hydroxynonenal, 1-[2-(1-adamantyl)ethyl]-1-pentyl-3-[3-(4-pyridyl)propyl]urea, or gingerol.

22. The composition of claim 21, wherein said capsaicinoid is selected from the group consisting of: capsaicin, dihydrocapsaicin, nordihydrocapsaicin, homodihydrocapsaicin, homocapsaicin, nonivamide, pseudocapsaicin, resiniferatoxin, tinyatoxin, capsiate, dihydrocapsiate, nordihydrocapsiate, norcapsaicin, capsiconiate, dihydrocapsiconiate and other coniferyl esters, capsiconinoid, and 3-hydroxyacetanilide.

23. The composition of claim 20, wherein said TRPA1 channel activator is allyl isothiocyanate, gingerol, cinnamaldehyde, acrolein, farnesyl thiosalicylic acid, Δ₉-tetrahydrocannabinol, eugenol, a shogaol, a sanshool, methyl salicylate, allicin, diallyl sulfide, diallyl disulfide, diallyl trisulfide, or farnesyl thioacetic acid.

24. The composition of claim 20, wherein said ASIC channel activator comprises acetic acid, phosphoric acid, citric acid, malic acid, succinic acid, lactic acid, tartaric acid, fumaric acid, or ascorbic acid.

25. The composition of any one of claims 20-24, wherein said TRPV1 channel activator, TRPA1 channel activator, ASIC channel activator, or a combination thereof is present from about 0.001% to about 10% (w/w) or about 0.001% to about 10% (v/v).

26. The composition of any one of claims 1-25, wherein said composition is capable of reducing gastrointestinal side effects.

27. A composition formulated for oral administration to a subject, said composition comprising an effective amount of an ion channel activator and a pharmaceutically acceptable excipient, wherein upon administration, the ion channel activator has a residence time of greater than about 5 seconds in the mouth of the subject.

28. The composition of claim 27, wherein said composition comprises a plurality of ion channel activators.

29. The composition of any one of claims 27-28, wherein said composition comprises two ion channel activators.

30. The composition of any one of claims 27-29, wherein said composition comprises an ion channel activator and a plurality of pharmaceutically acceptable excipients.

31. The composition of any one of claims 27-30, wherein said ion channel activator has a residence time of greater than about 60 seconds in the mouth of a subject.

32. The composition of any one of claims 27-31, wherein said composition further comprises a formulation base.

33. The composition of claim 32, wherein said formulation base comprises an oil and a lipophilic additive.

34. The composition of claim 33, wherein said oil is selected from the group consisting of: vegetable oil, mineral oil, soya oil, sunflower oil, corn oil, olive oil, nut oil, and liquid paraffin.

35. The composition of claim 33, wherein said lipophilic additive is selected from the group consisting of: polyethylene glycol, fatty acid mono-, di-, or triglycerides, palmitic acid, stearic acid, behenic acid, polyethylene glycol fatty acid esters, candelilla wax, carnauba wax, polyethylene oxide wax, and petroleum wax.

36. The composition of any one of claims 27-35, wherein the composition further comprises a coloring agent, a dissolving agent, a flavoring agent, a sweetener, a viscosity modifier, an electrolyte, a vitamin, a mineral, an antioxidant, or a preservative.

37. The composition of any one of claims 27-36, wherein said composition is formulated as a liquid.

38. The composition of claim 37, wherein said liquid is selected from the group consisting of emulsions, microemulsions, solutions, suspensions, syrups, linctuses, drops, sprays, and elixirs

39. The composition of any one of claims 27-36, wherein said composition is formulated as a solid.

40. The composition of claim 39, wherein said solid is selected from the group consisting of tablets, capsules, powders, crystals, pastes, gels, lozenges, gums, candies, chews, foodstuffs, dissolving strips, films, and semi-solid formulations.

41. The composition of claim 40, wherein said capsule is a hard or soft capsule.

42. The composition of any one of claims 27-41, wherein the ion channel activator comprises a TRPV1 channel activator, a TRPA1 channel activator, an ASIC channel activator, or a combination thereof.

43. The composition of claim 42, wherein said TRPV1 channel activator is a capsaicinoid, a capsinoid, oleoylethanolamide, N-oleoyldopamine, 3-methyl-N-oleoyldopamine, oleamide, capsiate, a 1-monoacylglycerol having C18 and C20 unsaturated and C8-C12 saturated fatty acid, a 2-monoacylglycerol having C18 and C20 unsaturated fatty acids, miogadial, miogatrial, polygodial, a terpenoid with an alpha,beta-unsaturated 1,4-dialdehyde moiety, sanshool, evodiamine, acesulfame-K, cyclamate, CuSO₄, ZnSO₄, FeSO₄, arvanil, anandamide, N-arachidonoyl-dopamine, flufenamic acid dopamide, a dopamine amide of fenamic acid, 4-hydroxynonenal, 1-[2-(1-adamantyl)ethyl]-1-pentyl-3-[3-(4-pyridyl)propyl]urea, or gingerol.

44. The composition of claim 43, wherein said capsaicinoid is selected from the group consisting of: capsaicin, dihydrocapsaicin, nordihydrocapsaicin, homodihydrocapsaicin, homocapsaicin, nonivamide, pseudocapsaicin, resiniferatoxin, tinyatoxin, capsiate, dihydrocapsiate, nordihydrocapsiate, norcapsaicin, capsiconiate, dihydrocapsiconiate and other coniferyl esters, capsiconinoid, and 3-hydroxyacetanilide.

45. The composition of claim 42, wherein said TRPA1 channel activator is allyl isothiocyanate, gingerol, cinnamaldehyde, acrolein, farnesyl thiosalicylic acid, Δ₉-tetrahydrocannabinol, eugenol, a shogaol, a sanshool, methyl salicylate, allicin, diallyl sulfide, diallyl disulfide, diallyl trisulfide, or farnesyl thioacetic acid.

46. The composition of claim 42, wherein said ASIC channel activator comprises acetic acid, phosphoric acid, citric acid, malic acid, succinic acid, lactic acid, tartaric acid, fumaric acid, or ascorbic acid.

47. The composition of any one of claims 27-46, wherein said TRPV1 channel activator, TRPA1 channel activator, ASIC channel activator, or a combination thereof is present from about 0.001% to about 10% (w/w) or about 0.001% to about 10% (v/v).

48. The composition of any one of claims 27-47, wherein said ion channel activator is ingested by the subject.

49. The composition of any one of claims 27-48, wherein said ion channel activator is held in the mouth by the subject.

50. The composition of any one of claims 27-49, wherein said ion channel activator is dissolved in the mouth of the subject or chewed by the subject prior to swallowing.

51. A composition for use in a method for treating a painful muscle contraction in a subject in need thereof, said composition comprising an effective amount of an ion channel activator and a pharmaceutically acceptable excipient.

52. The composition of claim 51, wherein said painful muscle contraction is a muscle contraction of the head or neck.

53. The composition of any one of claims 51-52, wherein said painful muscle contraction is associated with tension headache, cluster headache, or migraine headache.

54. A composition for use in a method for treating tactile sensitivity in a subject in need thereof, said composition comprising an effective amount of an ion channel activator and a pharmaceutically acceptable excipient.

55. The composition of claim 54, wherein said tactile sensitivity is associated with autism, dyspraxia, neuralgia, anxiety disorders, venomous bites, or venomous stings.

56. The composition of claim 55, wherein said anxiety disorder is selected from the group consisting of panic disorder, obsessive-compulsive disorder (OCD), post-traumatic stress disorder (PTSD), social anxiety disorder, phobia, and generalized anxiety disorder (GAD).

57. A composition for use in a method for treating a dystonia in a subject in need thereof, said composition comprising an effective amount of an ion channel activator and a pharmaceutically acceptable excipient.

58. The composition of claim 57, wherein said dystonia is selected from the group consisting of: focal dystonia, blepharospasm, cervical dystonia, cranial dystonia, laryngeal dystonia, and hand dystonia.

59. A composition for use in a method for treating a peripheral nervous system (PNS) condition in a subject in need thereof, said composition comprising an effective amount of an ion channel activator and a pharmaceutically acceptable excipient to said subject.

60. The composition of claim 59, wherein said PNS condition is selected from the group consisting of: cramp fasciculation syndrome, Isaacs' Syndrome or neuromyotonia, peripheral neuropathy, carpal tunnel syndrome, and Epstein-Barr virus infection.

61. A composition for use in a method for treating a throat condition in a subject in need thereof, said composition comprising an effective amount of an ion channel activator and a pharmaceutically acceptable excipient.

62. The composition of claim 61, wherein said throat condition is associated with chemical injury, cancer, surgical injury, or pathogen infection.

63. The composition of any one of claims 61-62, wherein said throat condition is selected from the group consisting of: acid reflux, laryngospasm, dysphagia, and spasmodic dysphonias.

64. A composition for use in a method for treating a condition associated with an electrolyte imbalance or vitamin deficiency in a subject in need thereof, said composition comprising an effective amount of an ion channel activator and a pharmaceutically acceptable excipient.

65. The composition of claim 64, wherein said condition is selected from the group consisting of: hyponatremia, hypocalcemia, hypomagnesemia, kidney disease, rickets, calcium or magnesium deficiency, thiamine deficiency, hypoparathyroidism, medullary cystic disease, and adrenocortical carcinoma.

66. A composition for use in a method for treating a central nervous system (CNS) condition in a subject in need thereof, said composition comprising an effective amount an ion channel activator and a pharmaceutically acceptable excipient.

67. The composition of claim 68, wherein said CNS condition is associated with a tumor.

68. The composition of any one of claims 66-67, wherein said CNS condition is selected from the group consisting of: multiple sclerosis, cerebral palsy, stroke, motor neuron disease, spinal injury, and stenosis.

69. A composition for use in a method for treating a muscle condition or disorder in a subject in need thereof, said composition comprising an effective amount of an ion channel activator and a pharmaceutically acceptable excipient.

70. The composition of claim 69, wherein said muscle condition or disorder is associated with muscle pain, muscle spasms, muscle cramps, fasciculations, or any combination thereof.

71. The composition of any one of claims 69-70, wherein said muscle condition or disorder is a neuromuscular disorder.

72. The composition of any one of claims 69-71, wherein said muscle condition or disorder is muscle pain, muscle spasms, spasticity, or fasciculations associated with motor neuron disease.

73. The composition of claim 72, wherein said motor neuron disease is selected from the group consisting of amyotrophic lateral sclerosis, primary lateral sclerosis, progressive muscular atrophy, progressive bulbar palsy, pseudobulbar palsy, spinal muscular atrophy, progressive spinobulbar muscular atrophy, and post-polio syndrome.

74. The composition of any one of claims 69-73, wherein said muscle condition or disorder is associated with treatment of said subject with dialysis, diuretics, β-blockers, statins, fibrates, β2-agonists, ACE inhibitors, ARBs, anti-psychotic medications, or any combination thereof.

75. The composition of claim 74, wherein said muscle condition or disorder is associated with treatment of said subject with statins and fibrates.

76. The composition of any one of claims 69-75, wherein said muscle condition or disorder occurs in one or more skeletal muscles.

77. The composition of any one of claims 69-76, wherein said muscle condition or disorder is refractory to an approved treatment.

78. The composition of claim 77, wherein said approved treatment is botox, cyclopenzaprine, orphenadrine, baclofen, or any combination thereof.

79. The composition of any one of claims 69-78, wherein said muscle condition or disorder involves muscle claudication pain.

80. The composition of claim 79, wherein said muscle claudication pain is associated with inactivity, restriction, economy class syndrome, paralysis, peripheral artery disease, or immobilization.

81. A composition for use in a method for treating a respiratory condition in a subject in need thereof, said composition comprising an effective amount of an ion channel activator and a pharmaceutically acceptable excipient.

82. The composition of claim 78, wherein said respiratory condition comprises asthma, chronic obstructive pulmonary disease, bronchitis, emphysema, pneumonia, cystic fibrosis, pleural cavity diseases, influenza, or cold.

83. A composition for use in a method for treating cough in a subject in need thereof, said composition comprising an effective amount of an ion channel activator and a pharmaceutically acceptable excipient.

84. The composition of claim 83, wherein said cough is caused by a respiratory condition, exposure to allergens or chemical irritants, or inflammation.

85. A composition for use in a method for treating sarcoidosis in a subject in need thereof, said composition comprising an effective amount of an ion channel activator and a pharmaceutically acceptable excipient.

86. A composition for use in a method for treating a connective tissue disease in a subject in need thereof, said composition comprising an effective amount of an ion channel activator and a pharmaceutically acceptable excipient.

87. The composition of claim 86, wherein said connective tissue disease is selected from the group consisting of Ehlers-Danlos syndrome, epidermolysis bullosa, Marfan syndrome, osteogenesis imperfect, arthritis, scleroderma, Sjögren's syndrome, lupus, vasculitis, mixed connective tissue disease, cellulitis, polymyositis, and dermatomyositis.

88. The composition of claim 87, wherein said arthritis is rheumatoid arthritis, osteoarthritis, gout, or psioratic arthritis, or wherein said vasculitis is Wegener's granulomatosis or Churg-Strauss Syndrome.

89. The composition of any one of claims 51-88, wherein said TRPV1 channel activator is a capsaicinoid, a capsinoid, oleoylethanolamide, N-oleoyldopamine, 3-methyl-N-oleoyldopamine, oleamide, capsiate, a 1-monoacylglycerol having C18 and C20 unsaturated and C8-C12 saturated fatty acid, a 2-monoacylglycerol having C18 and C20 unsaturated fatty acids, miogadial, miogatrial, polygodial, a terpenoid with an alpha,beta-unsaturated 1,4-dialdehyde moiety, sanshool, evodiamine, acesulfame-K, cyclamate, CuSO₄, ZnSO₄, FeSO₄, arvanil, anandamide, N-arachidonoyl-dopamine, flufenamic acid dopamide, a dopamine amide of fenamic acid, 4-hydroxynonenal, 1-[2-(1-adamantyl)ethyl]-1-pentyl-3-[3-(4-pyridyl)propyl]urea, or gingerol.

90. The composition of any one of claims 51-88, wherein said TRPA1 channel activator is allyl isothiocyanate, gingerol, cinnamaldehyde, acrolein, farnesyl thiosalicylic acid, Δ₉-tetrahydrocannabinol, eugenol, a shogaol, a sanshool, allicin, diallyl sulfide, diallyl disulfide, diallyl trisulfide, or farnesyl thioacetic acid.

91. The composition of any one of claims 51-88, wherein said ASIC channel activator comprises acetic acid, phosphoric acid, citric acid, malic acid, succinic acid, lactic acid, tartaric acid, fumaric acid, or ascorbic acid.

92. The composition of any one of claims 51-88, wherein said composition is formulated as a liquid.

93. The composition of claim 93, wherein said liquid is selected from the group consisting of emulsions, microemulsions, solutions, suspensions, syrups, linctuses, drops, sprays, and elixirs.

94. The composition of any one of claims 51-88, wherein said composition is formulated as a solid.

95. The composition of claim 94, wherein said solid is selected from the group consisting of tablets, capsules, powders, crystals, pastes, gels, lozenges, gums, candies, chews, foodstuffs, dissolving strips, films, and semi-solid formulations.

96. The composition of claim 95, wherein said capsule is a hard or soft capsule.

97. A composition for use in a method for treating a subject for unwanted or abnormal muscle contraction or absence of a normal muscle contraction comprising:
acquiring knowledge of a result of a test for the efficacy of the administration of a test aliquot of a composition comprising an ion channel activator for alleviation of test muscle contraction in said subject;
administering an amount of a composition comprising an ion channel activator sufficient to alleviate unwanted or abnormal muscle contraction or absence of a normal muscle contraction to said subject.

98. The composition of claim 97, wherein said muscle contraction comprises a muscle cramp, muscle spasm, dystonia, or fasciculation.

99. The composition of any one of claims 97-98, wherein said muscle contraction occurs in a skeletal muscle or smooth muscle.

100. The composition of claim 97, wherein the test muscle contraction is a test muscle cramp or a test muscle spasm.

101. The composition of claim 97, wherein the subject has a central nervous system disorder or injury.

102. The composition of claim 97, wherein the subject has been diagnosed with or identified as having multiple sclerosis.

103. The composition of claim 97, wherein the subject has been diagnosed with or identified as having dystonia.

104. The composition of claim 97, wherein the subject has been diagnosed with or identified as having spinal cord spasticity.

105. The composition of claim 97, wherein said subject has been diagnosed with or identified as having a disorder associated with muscle cramps, muscle pain, muscle spasms, spasticity, or fasciculations.

106. The composition of claim 97, comprising directly acquiring said knowledge.

107. The composition of claim 97, further comprising performing said test.

108. The composition of claim 97, wherein said muscle contraction being selected, treated, or diagnosed comprises a contraction in a muscle other than a muscle that is contracted in the test muscle contraction.

109. The composition of claim 97, wherein said test muscle contraction comprises a contraction in a muscle of the foot and the muscle cramp comprises a cramp in a muscle other than the foot.

110. The composition of claim 97, wherein said muscle contraction is not induced by applied electrical stimulation.

111. The composition of claim 97, wherein said muscle contraction is a night cramp.

112. The composition of claim 97, wherein said muscle contraction is associated with multiple sclerosis.

113. The composition of claim 97, wherein said muscle contraction is associated with spinal cord spasticity.

114. The composition of claim 97, wherein said muscle contraction is associated with dystonia.

115. The composition of claim 97, wherein said test comprises inducing said test muscle cramp by application of electrical stimulation.

116. The composition of claim 97, wherein said test comprises determining that a muscle contraction can be induced in a subject by application of electrical stimulation.

117. The composition of claim 97, wherein said test comprises:
a) administering the test aliquot of the composition to said subject;
b) inducing a test muscle contraction; and
c) evaluating the effect of administering the test aliquot of the composition on test muscle contraction.

118. The composition of claim 117, wherein step a is performed before step b.

119. The composition of claim 117, wherein step a is performed after step b.

120. The composition of any one of claims 97-119, wherein said ion channel activator comprises a TRPV1 channel activator, a TRPA1 channel activator, an ASIC channel activator, or a combination thereof.

121. The composition of claim 120, wherein said TRPV1 channel activator is a capsaicinoid, a capsinoid, oleoylethanolamide, N-oleoyldopamine, 3-methyl-N-oleoyldopamine, oleamide, capsiate, a 1-monoacylglycerol having C18 and C20 unsaturated and C8-C12 saturated fatty acid, a 2-monoacylglycerol having C18 and C20 unsaturated fatty acids, miogadial, miogatrial, polygodial, a terpenoid with an alpha,beta-unsaturated 1,4-dialdehyde moiety, sanshool, evodiamine, acesulfame-K, cyclamate, CuSO₄, ZnSO₄, FeSO₄, arvanil, anandamide, N-arachidonoyl-dopamine, flufenamic acid dopamide, a dopamine amide of fenamic acid, 4-hydroxynonenal, 1-[2-(1-adamantyl)ethyl]-1-pentyl-3-[3-(4-pyridyl)propyl]urea, or gingerol.

122. The composition of claim 120, wherein said TRPA1 channel activator is allyl isothiocyanate, a gingerol, cinnamaldehyde, acrolein, farnesyl thiosalicylic acid, Δ₉-tetrahydrocannabinol, eugenol, a shogaol, a sanshool, allicin, diallyl sulfide, diallyl disulfide, diallyl trisulfide, or farnesyl thioacetic acid.

123. The composition of claim 120, wherein said ASIC channel activator comprises acetic acid, phosphoric acid, citric acid, malic acid, succinic acid, lactic acid, tartaric acid, fumaric acid, or ascorbic acid.

124. The composition of claim 120, wherein said TRPV1 channel activator, TRPA1 channel activator, ASIC channel activator, or combination thereof is present from about 0.001% to about 10% (w/w) or about 0.001% to about 10% (v/v).
